(19) United States
(12) Patent Application Publication
(10) Pub. No.: US 2020/0115442 A1
(43) Pub. Date: Apr. 16, 2020

(54) PROTEIN AQUEOUS SUSPENSION PREPARATION

(71) Applicant: TERUMO KABUSHIKI KAISHA, Tokyo (JP)

(72) Inventors: Shunsuke IZAKI, Takasaki-shi (JP); Tomoaki KIMOTO, Fujisawa-shi (JP); Kenji HANDA, Hiratsuka-shi (JP); Shiuhei MIEDA, Atsugi-shi (JP); Kentaro SHIRAKI, Tsukuba-shi (JP); Takaaki KURINOMARU, Tsukuba-shi (JP); Takuya MARUYAMA, Tsukuba-shi (JP)

(73) Assignee: TERUMO KABUSHIKI KAISHA, Tokyo (JP)

(21) Appl. No.: 16/657,215

(22) Filed: Oct. 18, 2019

Related U.S. Application Data
(62) Division of application No. 15/082,435, filed on Mar. 28, 2016, now abandoned.
Application No. 15/082,435 is a continuation of application No. PCT/JP2014/078667, filed on Oct. 28, 2014.

(30) Foreign Application Priority Data
Oct. 28, 2013 (JP) 2013-223775

(51) Int. Cl. C07K 16/18; A61K 47/64; C07K 16/24; A61K 39/395; C07K 16/28; C07K 16/42; C07K 16/32; C07K 16/00; C07K 14/705; A61M 5/315; A61M 5/19; A61K 38/50; A61K 38/38; A61K 38/22; A61K 38/21; A61K 38/17

(52) U.S. Cl. CPC C07K 16/18; A61K 38/00; A61K 47/645; C07K 16/241; A61K 39/39591; C07K 16/2863; C07K 16/4291; C07K 16/2887; C07K 16/32; C07K 16/00; A61K 39/395; C07K 14/70578; A61M 5/31596; A61M 5/19; A61K 39/39558; A61K 39/3955; A61K 38/50; A61K 38/385; A61K 38/2242; A61K 38/22; A61K 38/212; A61K 38/1793; C07K 2317/55; A61K 47/6455

(57) ABSTRACT

Disclosed is a protein aqueous suspension preparation containing a protein and a polyamino acid, the protein and the polyamino acid having a surface charge in a buffer and forming a complex suspended in the buffer, wherein the absolute value of the difference between pH of the buffer and isoelectric point pI of the protein is in the range of from 0.5 to 4.0. Also disclosed are a method of preparing a protein aqueous suspension preparation and a prefilled syringe containing a concentrated protein aqueous suspension preparation. The protein can exhibit at least one of shaking stress resistance, fluidity enhancement, oxidation resistance, thermal stability, and aggregation inhibitory properties.

### CROSS-REFERENCES TO RELATED APPLICATIONS

This application is a divisional application of U.S. application Ser. No. 15/082,435 filed Mar. 28, 2016, which is a continuation of International Application No. PCT/JP2014/078667 filed on Oct. 28, 2014, and claims priority to Japanese Application No. 2013-223775 filed on Oct. 28, 2013, the entire content of all three of which is incorporated herein by reference.

### TECHNICAL FIELD

Disclosed is an aqueous suspension preparation of a complex of a protein and a polyamino acid wherein in the complex, the protein has at least one of shaking stress resistance, fluidity enhancement, oxidation resistance, thermal stability, and aggregation inhibitory properties and its activity is not deteriorated when concentrated. In addition, the disclosure relates to a method of preparing a protein aqueous suspension preparation and a prefilled syringe containing a concentrated protein aqueous suspension preparation.

### BACKGROUND DISCUSSION

Owing to the progress of genetic recombination technique, there has been an increasing number of chances that a variety of proteins are provided as drugs. Particularly, there has been a remarkable technological progress concerning antibodies, where chimera antibodies, humanized antibodies and, further, antibodies based on application of sugar chain alteration techniques have been put to practical use, and a number of antibody drugs have produced great therapeutic effects. While protein drugs were conventionally provided as freeze-dried preparations to be dissolved when put to use, they have been coming in recent years to be provided as aqueous preparations which can be used more easily. As for administration, protein drugs were conventionally administered by intravenous injection, but an increasing number of protein drugs have been coming to be administered by subcutaneous injection, taking the patient's convenience into account. In subcutaneous injection, the dose volume must be reduced as compared to intravenous injection, so that it is indispensable to raise the concentration of the protein. In addition, antibody drugs are used in large doses, so that a greater increase in concentration is required in the case of subcutaneous injection of an antibody drug. Further, in recent years, attempts have been made to enhance the concentration for the purpose of reducing the administration frequency, and commercial pharmaceutical preparations that have been provided include those which have a protein concentration of not less than 100 mg/mL.

However, proteins, which are biopolymers, are intrinsically vulnerable to physical stresses such as shaking and chemical stresses such as oxidation; particularly, proteins are more susceptible to denaturation when provided as aqueous preparations as compared to freeze-dried preparations. The denatured proteins often lose their original activity and form aggregates or association products, the reaction being often irreversible. Such formation of aggregates is more likely to occur as the protein concentration is higher, and the possibility of aggregate formation increases particularly during long-time storage and during transportation, because of an increase in the number of chances of exposure to a gas-liquid interface and interaction between protein molecules due to shaking or the like. The formation of aggregates lowers the efficacy of the protein drugs and, further, threatens the safety of the protein drugs. Specifically, the formation of aggregates has the possibility of inducing an undesired immunoreaction such as production of an anti-drug antibody after administration (Non-patent Document 1). For this reason, the FDA in the United States and the EMEA in Europe have set guidelines as to evaluation of immunogenicity and stimulated countermeasures. Besides, among low-concentration preparations of proteins such as enzymes and cytokines, there are those which are liable to form aggregates, depending on the kind or preparation thereof, and not a few of them demand care in handling at the time of use, such as avoiding severe shaking of the preparation, passing the preparation through a filter, etc. at the time of dissolution or administration.

From such a background, it is an extremely important task to enhance stability of proteins against various kinds of stress, and there is a need for creation of a highly versatile stabilization technique which is applicable particularly to proteins in the state of high concentration and to proteins liable to form aggregates.

In view of the above, a large number of investigations have been made in order to enhance the stabilization of the proteins mainly by using various additives. In Patent Document 1, a method of stabilizing proteins by addition of an amino acid ester or a polyamine is disclosed. Furthermore, in Patent Document 2, there is disclosed a method of stabilizing a protein against thermal stress by coexistence of the protein with polyethylene glycol and a specific amino acid such as alanine, arginine, glutamic acid, etc. or their derivatives.

On the other hand, investigations of complexes of a protein and a polyamino acid as a drug delivery system have also been made. In Patent Document 3, it is disclosed that complexes of a hydrophobicized polyamino acid and an antigen protein or antigen polypeptide can be utilized as an immunoadjuvant for enhancing immunogenicity, aimed at imparting stability and functionality in a living body. In addition, in Patent Document 4, complexes using a polyamino acid as one of cationic polymers is disclosed as a technology which reduces interactions between a contained protein and a polymer matrix constituting a delivery system and which is incorporated into a system exhibiting excellent sustained release properties. However, the document does not discuss the stability of aqueous suspension preparations containing these complexes during transportation or during storage.

In Patent Document 5, “a method of making an antibody formulation comprising combining an antibody with a polycation” is described in claim 1. It is described in claim 4 that “the polycation is selected from the group consisting of polylysine, polyarginine, polyornithine, polyhistidine, and cationic polysaccharides, or mixtures thereof.” It is described in claim 24 that “the formulation has a greater solubility when combined with the polycation composition as compared to the solubility of the antibody formulation in the absence of polycations,” and it is described in claim 25 that “the formulation has an increased shelf-life as compared to such an antibody formulation that has not been combined with a polycation composition.” However, there is no description about formation of solid particles, which are a complex of an antibody and a polyamino acid, or about combinations of an antibody and a polyanion. In Patent Document 5, it is described that “in one embodiment, the antibody has a greater solubility in water than a composition containing the antibody or Fc-fusion molecule in the absence of a polycation” (see Non-patent Document 1: Schellekens, H. Clin. Therapeutics, 24(11): 1720-1740. 2002
Patent Document 1: Japanese Patent No. 3976257
Patent Document 2: Japanese Patent No. 5119545
Patent Document 3: PCT Patent Publication No. WO2010/110455
Patent Document 4: Japanese Translations of PCT for Patent No. 1995-503700
Patent Document 5: Japanese Translations of PCT for Patent No. 2010-536786

### SUMMARY

In consideration of the above-mentioned circumstances, one aspect is to provide an aqueous suspension preparation containing a protein, being excellent in stability during transportation and during storage and being excellent in handleability when put to use. Studies on a variety of proteins have been undertaken and it has been determined that a protein and a polyamino acid form a complex which is suspended in a buffer, and, in the complex, the protein has at least one of shaking stress resistance, fluidity enhancement, oxidation resistance, thermal stability, and aggregation inhibitory properties, thereby being enhanced in stability.

It has further been determined that a method including a step of combining a protein with a polyamino acid causes the protein to have shaking stress resistance and to have enhanced stability.

Various embodiments are provided as follows:

(1) A protein aqueous suspension preparation comprising a protein and a polyamino acid, said protein and said polyamino acid having a surface charge in a buffer and forming a complex suspended in the buffer, wherein the absolute value of the difference between pH of the buffer and isoelectric point pI of the protein is in the range of from 0.5 to 4.0.

(2) The protein aqueous suspension preparation as described in (1), wherein the protein has a positive surface charge and the polyamino acid is an anionic polyamino acid.

(3) The protein aqueous suspension preparation as described in (1), wherein the protein has a negative surface charge and the polyamino acid is a cationic polyamino acid.

(4) The protein aqueous suspension preparation as described in (3), wherein the cationic polyamino acid is at least one selected from group consisting of polylysine, polyarginine, polyhistidine, and their water-soluble salts, and the anionic polyamino acid is at least one selected from group consisting of polyglutamic acid, polyaspartic acid, and their water-soluble salts.

(5) The protein aqueous suspension preparation as described in (1), wherein a molecular weight of the polyamino acid is in a range from 0.5 kDa to 1000 kDa.

(6) The protein aqueous suspension preparation as described in (1), wherein a molecular weight of the protein is in a range from 3 kDa to 670 kDa.

(7) The protein aqueous suspension preparation as described in (1), wherein the protein is at least one of an enzyme, a cytokine, a hormone, an antibody, an antibody fragment, and a fusion protein.

(8) The protein aqueous suspension preparation as described in (7), wherein the protein is an antibody or an antibody fragment.

(9) The protein aqueous suspension preparation as described in (1), wherein the absolute value of the difference between pH of the buffer and isoelectric point pI of the protein is in the range of from 1.0 to 4.0.

(10) The protein aqueous suspension preparation as described in (1), wherein the absolute value of the difference between pH of the buffer and isoelectric point pI of the protein is in the range of from 1.8 to 3.0.

(11) A method of preparing a protein aqueous suspension preparation comprising forming a complex of a protein having a surface charge and a polyamino acid having a surface charge in a buffer, wherein the absolute value of the difference between pH of the buffer and isoelectric point pI of the protein is in the range of from 0.5 to 4.0, and removing water or buffer from the protein aqueous suspension preparation to increase the concentration of the protein in the protein aqueous suspension preparation.

(12) The method of preparing a protein aqueous suspension preparation as described in (11), wherein the removal of water or buffer increases the concentration of the protein from 3 to 10 times the original concentration in the protein aqueous suspension preparation.

(13) The method of preparing a protein aqueous suspension preparation as described in (11), wherein the absolute value of the difference between pH of the buffer and isoelectric point pI of the protein is in the range of from 1.8 to 3.0.

(14) The method of preparing a protein aqueous suspension preparation as described in (11), wherein the protein is an antibody or an antibody fragment.

(15) A prefilled syringe comprising an axially extending sheath possessing a proximal end and a distal end, the sheath possessing an interior divided into a first chamber and a second chamber by a slidable gasket positioned axially between the first and second chambers, and a plunger possessing a distal end portion positioned in the interior of the sheath at a position proximal of the gasket, the plunger being axially movable relative to the sheath in a distal direction, the first chamber containing a protein aqueous suspension preparation comprised of a protein and a polyamino acid, said protein and said polyamino acid having a surface charge in a buffer and forming a complex suspended in the buffer, wherein the protein aqueous suspension preparation has been concentrated by removal of water or buffer, the second chamber containing an aqueous electrolyte solution, the sheath being configured so that axial movement of the plunger in the distal direction causes the first and second chambers to communicate with one another so that the protein aqueous suspension of the first chamber and the aqueous electrolyte solution of the second chamber mix together to dissolve the complex and produce a resulting protein solution that is dispensable from the distal end of the sheath.

(16) The prefilled syringe as described in (15), wherein the sheath includes an enlarged diameter section so that when the gasket axially overlaps the enlarged diameter section, the first and second chambers communicate with one another so that the protein aqueous suspension and the aqueous electrolyte solution are mixed to dissolve the complex.

(17) The prefilled syringe as described in (15), wherein the first chamber is closer to the distal end of the sheath and the second chamber is closer to the proximal end of the sheath.

(18) The prefilled syringe as described in (15), wherein the aqueous electrolyte solution is an aqueous solution of sodium chloride.

(19) The prefilled syringe as described in (15), wherein the protein is an antibody or an antibody fragment.

(20) The prefilled syringe as described in (15), wherein the polyamino acid is at least one selected from group consisting of polylysine, polyarginine, polyhistidine, polyglutamic acid, polyaspartic acid, and their water-soluble salts.

In the disclosed aqueous suspension preparation, a protein and a polyamino acid that have a surface charge in a buffer form a complex to be suspended in the buffer, and the protein in the complex is not deteriorated when concentrated as it has at least one of shaking stress resistance, fluidity enhancement, oxidation resistance, thermal resistance, and aggregation inhibitory properties. Specifically, the protein and the polyamino acid form a complex, whereby the protein is stabilized and can be concentrated by removing water from the aqueous suspension. The aqueous suspension preparation is excellent in stability during transportation and during storage, and is excellent in handleability when put to use. In general, for stabilization of protein drugs, use is made of additives such as excipients, surfactants, stabilizers, etc., and when these additives are used, a substantial research is required for optimizing the kinds, combinations, and amounts of the additives on the basis of each of the protein preparations. The disclosed aqueous suspension preparations can stabilize a protein by forming a complex of the protein with a polyamino acid, and eliminates the need for addition of additives that has conventionally been necessary. Besides, the disclosed aqueous suspension preparation does not need a complicated dissolving operation, which is needed for freeze-dried pharmaceutical preparations, and can be administered as it is or administered in the form of an aqueous liquid obtained through addition of an inorganic salt represented by sodium chloride. Furthermore, the disclosed aqueous suspension preparation is characterized by being low in viscosity even when it contains the protein in a high concentration. Therefore, the amount of the aqueous suspension preparation which would be left uselessly in a container when put to use can be reduced. Besides, when administered by use of a syringe, the aqueous suspension preparation can be administered with a weak force as compared to an aqueous solution containing the protein in the same concentration.

The disclosed aqueous suspension preparation has one of the above-mentioned characteristic features.

### BRIEF DESCRIPTION OF THE DRAWINGS

FIG. 1 depicts figures depicting states of addition of polylysine (Poly-K, molecular weight 4 kDa to 15 kDa) to L-asparaginase. In each of FIGS. 1) and 2) of FIG. 1, the sample on the right side is L-asparaginase in a MOPS buffer, and the sample on the left side is a sample in which polylysine was added to L-asparaginase in the MOPS buffer. 1) of FIG. 1 depicts an aqueous suspension in which a complex is formed in the left sample immediately after the addition. 2) depicts a state in which the complex formed in the left sample precipitates naturally after the lapse of a predetermined period of time. On the other hand, 1) and 2) demonstrate that no complex is formed in the right sample to which polylysine has not been added.

FIG. 2 depicts states after centrifugation of the same samples as those in FIG. 1. The left sample in 3) of FIG. 2 depicts a state in which the complex becomes pellet-like after centrifugation of the left sample in 2) of FIG. 1. The left sample in 4) of FIG. 2 depicts a state in which by removal of the buffer as supernatant after the centrifugation, the protein in the complex is concentrated to a concentration of 10 times that before the addition of polylysine. The right sample in 3) and 4) of FIG. 2 demonstrate that no change occurs after the centrifugation.

FIG. 3 depicts, in the left sample in 5) of FIG. 3, a state in which the whole body of the left sample in 4) of FIG. 2 has been shaken and the complex having been pellet-like has been thereby re-suspended. The right sample in 5) of FIG. 3 demonstrates that even when the whole body of the right sample in 4) of FIG. 2 is shaken, the state of absence of any complex formed is unchanged. 6-1) of FIG. 3 is a figure depicting a state after addition of NaCl to 5) of FIG. 3. The left sample in 6-1) of FIG. 3 depicts a state in which NaCl has been added to the left sample in 5) of FIG. 3 and the complex has been thereby re-dissolved. The right sample in 6-1) of FIG. 3 demonstrates that even when NaCl is added to the right sample in 5) of FIG. 3, the state of absence of any complex formed is unchanged. The left sample in 6-1) of FIG. 3 is in a re-solubilized state concentrated to a concentration of about 10 times that before the addition of polylysine, whereas the right sample in 6-1) of FIG. 3 depicts a state of being at a concentration comparable to that of the right sample in 5) of FIG. 3 or being diluted according to the addition of NaCl. In addition, the left sample in 6-2) of FIG. 3 demonstrates that even when a buffer, water or the like is added to the left sample in 6-1) of FIG. 3 to adjust the amount, the re-dissolved state is unchanged. The left sample in 6-2) of FIG. 3 depicts a state in which by addition of a buffer to the left sample in 6-1) of FIG. 3, a protein concentration comparable to that before the addition of polylysine is attained. The right sample in 6-3) of FIG. 3 depicts a state in which a supernatant has been removed from the right sample in 5) of FIG. 3 and, further, NaCl has been added to both the samples. The left sample in 6-3) of FIG. 3 is in a re-solubilized state concentrated to a concentration of about 10 times that before the addition of polylysine, in the same manner as the left sample in 6-1) of FIG. 3, whereas the right sample in 6-3) of FIG. 3 represents a state of being diluted according to the addition of NaCl, as compared to the state before the addition of polylysine, and, further, a state in which 90 mass % of the protein has been lost.

FIG. 4 is a graph depicting concentration factor of L-asparaginase in relation to the ratio in mixing part by mass of L-asparaginase and polylysine that are added to a MOPS buffer. The solid line represents the protein concentration (concentration factor), while the broken line represents activity ratio of protein. Both lines agree with each other, indicating that the activity of the protein is maintained after a complex is formed and then solubilized.

FIG. 5 is a graph depicting that maximum concentration factors of complexes of protein and polyamino acid are present where the absolute value of the difference between pH of a buffer and isoelectric point pI of the protein is in the range from 0.5 to 4.0.

FIG. 6 is a graph obtained in Test Example 13 to be described later by measurement of CD spectrum for Example 84 (broken line) in which an anti-TNFα monoclonal antibody-poly-L-glutamic acid complex (in MOPS buffer) is formed, concentrated and re-dissolved, and for Comparative Example 84 (solid line) containing no polyamino acid and not being concentrated, as a control liquid. The CD spectra of both samples agree with each other, indicating that a secondary structure of the protein is maintained unchanged after the complex is formed and then solubilized.

FIG. 7 is a graph obtained in Test example 14 to be described later by measurement of CD spectrum for Example 87 (broken line) in which an anti-TNFα monoclonal antibody-poly-L-glutamic acid complex (in MOPS buffer) is formed, concentrated and then re-dissolved, and for Comparative Example 87 (solid line) containing no polyamino acid and not being concentrated, as a control liquid. The CD spectra of both samples agree with each other, indicating that a secondary structure of the protein is maintained unchanged after the complex is formed and then solubilized.

FIG. 8 is a graph obtained in Test Example 15 to be described later by measurement of CD spectrum for Example 88 (broken line) in which an anti-TNFα monoclonal antibody-poly-L-glutamic acid complex (in citrate buffer) is formed, concentrated and then re-dissolved, and for Comparative Example 88 (solid line) containing no polyamino acid and not being concentrated, as a control liquid. The CD spectra of both samples agree with each other, indicating that a secondary structure of the concentrated protein is maintained unchanged when the complex is formed and then solubilized.

FIG. 9 is a graph obtained in Test Example 15-2 to be described later by measurement of CD spectrum for Example 88-2 (broken line) in which a human IgG-poly-L-glutamic acid complex (in citrate buffer) is formed, concentrated and then re-dissolved, and for Comparative Example 88-2 (solid line) containing no polyamino acid and not being concentrated, as a control liquid. The CD spectra of both samples agree with each other, indicating that a secondary structure of the concentrated protein is maintained unchanged when the complex is formed and then solubilized.

FIG. 10 is a graph depicting CD spectra measured in Test Example 26 to be described later, after shaking (broken line) of an anti-TNFα monoclonal antibody-poly-L-glutamic acid complex-containing aqueous suspension (Example 92), and before shaking (solid line) of a control liquid (Comparative Example 92) containing no polyamino acid. The CD spectrum obtained for the case in which shaking stress is applied to the anti-TNFα monoclonal antibody-poly-L-glutamic acid complex-containing aqueous suspension and for the case in which shaking stress is not applied agree with each other, indicating that a secondary structure of the protein is maintained even when shaking stress is applied to the protein in the complex.

FIG. 11 is a graph depicting CD spectra measured in Test Example 27 to be described later, after shaking (broken line) of an anti-IgE monoclonal antibody-poly-L-glutamic acid complex-containing aqueous suspension (Example 96), and before shaking (solid line) and after shaking (broken line) of a control liquid (Comparative Example 96) containing no polyamino acid. The CD spectrum for the case where shaking stress is applied to the control liquid and the CD spectrum for the case where shaking stress is not applied do not agree with each other, indicating that a change in secondary structure is generated. On the other hand, the CD spectrum for the case where shaking stress is applied to the anti-IgE monoclonal antibody-poly-L-glutamic acid complex-containing aqueous suspension and the CD spectrum for the case where shaking stress is not applied agree with each other, indicating that the secondary structure of the protein is maintained unchanged even when shaking stress is applied to the protein in the complex.

FIG. 12 is a graph depicting recovery rate of L-asparaginase from an L-asparaginase-polyallylamine complex (PAA-ASNase)-containing aqueous suspension (Control Example 111) and recovery rate of L-asparaginase from an L-asparaginase-polyethyleneimine complex (PEI-ASNase)-containing aqueous suspension (Control Example 112). While the recovery rates of L-asparaginase from the L-asparaginase-polylysine complex-containing aqueous suspension (refer to Examples 1 to 11) and from the L-asparaginase-polyethyleneimine complex-containing aqueous suspension were close to 100%, the recovery rate of L-asparaginase from the L-asparaginase-polyallylamine complex-containing aqueous suspension was significantly low. This demonstrates that like polylysine, polyethyleneimine forms a complex with L-asparaginase and can be concentrated and recovered.

FIG. 13 is a graph depicting the results of measurement of retained activity of L-asparaginase after shaking, for an L-asparaginase-polylysine complex-containing aqueous suspension (Example 114), an L-asparaginase-polyethyleneimine complex-containing aqueous suspension (Control Example 114), and an L-asparaginase MOPS buffer (pH 7.0) solution (Comparative Example 114, buffer). As compared to the retained activity in the L-asparaginase-polylysine complex-containing aqueous suspension (Example 114), the retained activity in the L-asparaginase-polyethyleneimine complex-containing aqueous suspension (Control Example 114) was significantly low, and was comparable to the retained activity in Comparative Example 114. In other words, it was depicted that the L-asparaginase-polyethyleneimine complex-containing aqueous suspension does not have a stabilizing effect against shaking stress.

FIG. 14 is a schematic view illustrating operations of a two-gas-chamber prefilled syringe during storage and at the time of dissolution.

FIG. 15(A) is a graph depicting variations in body weight of rats subjected to subcutaneous injection of a rat IgG citrate buffer (pH 5.0) solution (white circles) and a rat IgG-polyglutamic acid complex-containing aqueous suspension (black circles). The axis of ordinates represents the rat's body weight (g) and the axis of abscissas represents the number of days after injection. There was observed no significant difference in the rat's body weight between the rat IgG citrate buffer (pH 5.0) solution administration group (white circles) and the rat IgG-polyglutamic acid complex-containing aqueous suspension administration group (black circles). FIG. 15(B) is a graph obtained by measurement of weights of organs after one week and after two weeks from subcutaneous injection of a rat IgG citrate buffer (pH 5.0) solution and a rat IgG-polyglutamic acid complex-containing aqueous suspension into rats. There was observed no significant difference in any rat organ weight between the rat IgG citrate buffer (pH 5.0) solution administration group (white bars) and the rat IgG-polyglutamic acid complex-containing aqueous suspension administration group (black bars).

FIG. 16 is a graph depicting the transition of concentration of anti-IgE monoclonal antibody in plasma after subcutaneous injection of an anti-IgE monoclonal antibody solution (white circles) and an anti-IgE monoclonal antibody-polyglutamic acid complex-containing aqueous suspension (black circles) into rats. The axis of ordinates represents the concentration of anti-IgE monoclonal antibody in the rat plasma, and the axis of abscissas represents the number of days after injection. The plasma anti-IgE monoclonal antibody concentration-time curve for the anti-IgE monoclonal antibody solution administration group (white circles) and that for the anti-IgE monoclonal antibody-polyglutamic acid complex-containing aqueous suspension administration group (black circles) were approximate to each other.

### DETAILED DESCRIPTION

Illustrative embodiments are described specifically below by depicting examples of the protein-polyamino acid complex-containing aqueous suspension preparation, but the present invention is not limited to these examples.

A protein that can be used is not specifically restricted so long as it is a protein which is of high purity and has a surface charge in a buffer. Preferably, the protein is a protein having bioactivity, more particularly a protein originated from an organism such as vegetable, animal, microorganism, etc., or a protein produced by use of a genetic recombination technique, or the like. Proteins in more preferable form are proteins for medical use, such as enzymes, cytokines, hormones, antibodies, antibody fragments, fusion proteins, etc.

When the protein is an antibody or an antibody fragment, if the disclosed aqueous suspension preparation is used in applying these proteins to a human as a pharmaceutical preparation or a diagnostic agent, the protein can be transported and stored more stably, and can be easily administered to the human when put to use, ensuring high usefulness. The protein may have a sugar chain, which is hydrophilic and is considered to have some interaction in the disclosed complex-containing aqueous suspension preparation.

The high purity in the case of proteins for medical use means a drug level, or a level such as to be usable as a drug for humans. In the cases of other proteins, the high purity means a high purity of reagent grade; for example, a threshold of not more than 0.1% by weight in total amount of impurities may be mentioned.

A polyamino acid or its water-soluble salt is not particularly limited so long as it is a polyamino acid or its water-soluble salt that has a surface charge in a buffer. For the polyamino acid to be used in the disclosed complex or protein aqueous suspension preparation, a high purity of reagent grade, for example, a threshold of not more than 0.15% by weight in total amount of impurities may be mentioned.

Specifically, the polyamino acid is a cationic or anionic polyamino acid or a salt thereof. As the cationic polyamino acid, there may be mentioned sodium salts of polyglutamic acid, polyaspartic acid, etc., hydrochlorides of polyarginine, polylysine, polyornithine, polycitrulline, etc., and so on. As for the form of the polyamino acid of more preferable form, there may be mentioned, as examples of the cationic polyamino acid, poly-L-glutamic acid sodium salt, and poly-L-aspartic acid sodium salt, and there may be mentioned, as examples of the anionic polyamino acid, poly-L-arginine hydrochloride, poly-L-lysine hydrochloride, poly-L-arginine hydrobromide, and poly-L-lysine hydrobromide. The polyamino acid is a homopolymer or a copolymer or a mixture thereof. Preferably, the polyamino acid is a homopolymer. The polyamino acid preferably has a CH₂ group present between a main chain and a carboxyl group or amino group in a side chain. A 50% cell growth inhibitory concentration of the polyamino acid is preferably not less than 0.2%, more preferably not less than 1.0%.

A buffer refers to a solution having a buffering action on hydrogen ion concentration. The buffer is an aqueous solution so controlled that its pH is not largely varied even when a small amount of acid or base is added to the solution or when its concentration is somewhat changed. The buffer is not particularly limited so long as it is ordinarily in chemical use, but preferably is a buffer for biological use. As a buffer of more preferable form, there can be used buffers which are suitable for biosamples and which can be administered into living bodies, such as phosphate buffer, citrate buffer, MOPS buffer (3-(N-morpholino)propanesulfonic acid) buffer, PIPES (Piperazine-1,4-bis(2-ethanesulfonic acid)) buffer, Tris-HCl buffer, MES (2-Morpholinoethanesulfonic acid, monohydrate) buffer, HEPES (4-(2-Hydroxyethyl)-1-piperazine ethanesulfonic acid) buffer, glycine NaOH buffer, etc.

The disclosed complex has the following characteristic properties (1) to (4).

(1) The complex of a protein and a polyamino acid is obtained in a complex-containing aqueous suspension in which the protein and the polyamino acid form the complex in a buffer to be suspended in the buffer.

(2) The protein can be concentrated by forming the complex in the buffer and removing at least part of water from the complex-containing aqueous suspension obtained.

(3) The complex can be dissolved by adding a low-concentration electrolyte to the complex-containing aqueous suspension.

(4) The protein is stable in the complex, and its activity is not deteriorated when it is concentrated.

The aforesaid characteristic properties (1) to (3) will be described below, taking as an example a case where the protein is L-asparaginase and the polyamino acid is polylysine.

(1) The complex of a protein and a polyamino acid is obtained in a complex-containing aqueous suspension in which the protein and the polyamino acid form the complex in a buffer to be suspended in the buffer.

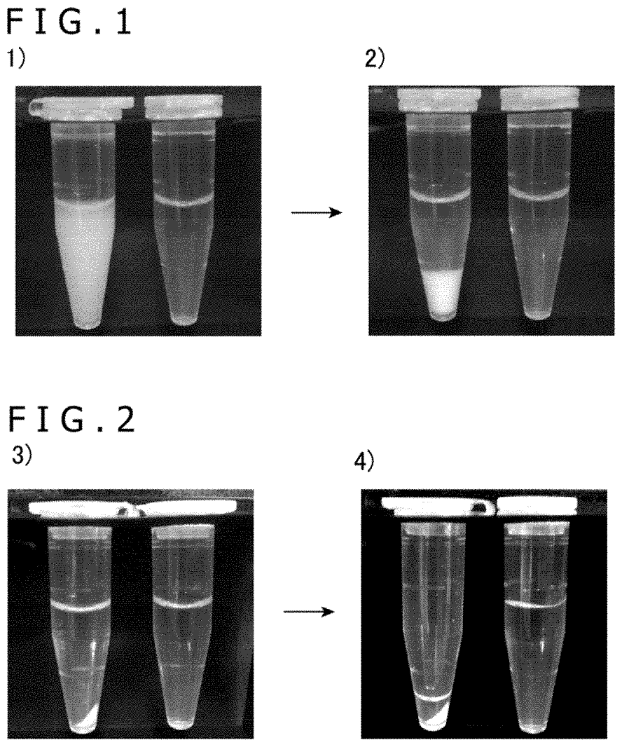

FIG. 1 depicts figures depicting states of addition of polylysine (Poly-K, molecular weight 4 kDa to 15 kDa) to L-asparaginase. The sample on the right side is L-asparaginase in a MOPS buffer, and the sample on the left side is a sample in which 0.05 part by mass of polylysine is added to 1 part by mass of L-asparaginase in the MOPS buffer. The left sample in 1) is a picture depicting an aqueous suspension in which a complex in the form of white solid particles is formed immediately after the addition. The left sample in 2) is a picture depicting a state in which the complex formed precipitates naturally after the lapse of a predetermined period of time. On the other hand, 1) and 2) depict that no complex is formed in the right sample to which polylysine has not been added.

FIG. 2 depicts the same samples as in FIG. 1. The left sample in 3) is a picture depicting a state in which the left sample in 2) of FIG. 1 is centrifuged and the complex is thereby turned into pellet-like form. The left sample in 4) is a picture depicting a state in which the buffer as a supernatant is removed and the complex is thereby concentrated to a concentration of 10 times the original concentration.

It is considered that part of the protein may remain in the supernatant, depending on the conditions at the time of formation of the complex.

(2) The protein can be concentrated by forming the complex in the buffer and removing at least part of water from the complex-containing aqueous suspension obtained.

As depicted in the left sample in 4) of FIG. 2, the protein-polyamino acid complex obtained makes it possible to obtain the complex retaining the concentrated protein by removing the buffer of the supernatant. The complex may be obtained by centrifugation, as depicted in the left sample in 3) of FIG. 2. The complex-containing aqueous suspension can be diluted to an arbitrary concentration by adding a buffer, water or the like thereto.

(3) The complex can be dissolved by adding a low-concentration electrolyte, which is an inorganic salt, to the complex-containing aqueous suspension. As examples of the electrolyte, there can be mentioned NaCl, KCl, CaCl₂, MgCl₂, etc., among which NaCl being the highest in biocompatibility is preferably selected. The concentration of the electrolyte is not particularly limited and is not more than 5% by weight, and the electrolyte can be used in a sufficient amount for dissolving the complex.

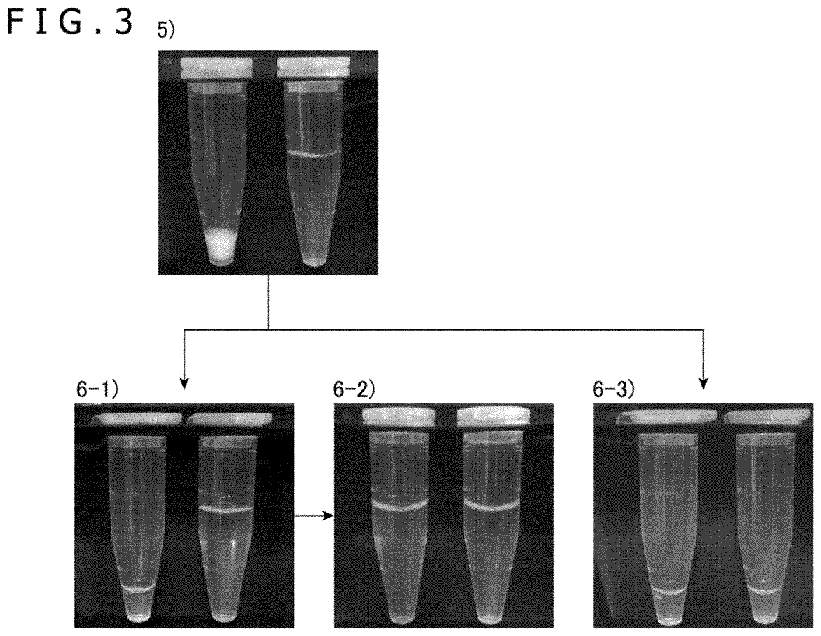

FIG. 3 depicts the same samples as in FIG. 2. The left sample in 5) of FIG. 3 is a picture depicting a state in which the whole body of the left sample in 4) of FIG. 2 is shaken and the complex having been pellet-like is thereby re-suspended. The right sample in 5) of FIG. 3 depicts that the state of absence of any complex formed remains unchanged even when the whole body of the sample is shaken, like the right sample in 3) and 4) in FIG. 2 in which no complex is formed.

The left sample in 6-1) of FIG. 3 depicts a state in which NaCl has been added to the left sample in 5) of FIG. 3 and the complex has been thereby re-dissolved. The right sample in 6-1) of FIG. 3 depicts that even when NaCl is added to the right sample in 5) of FIG. 3, the state of absence of any complex formed remains unchanged. The left sample in 6-1) of FIG. 3 is in a re-solubilized state concentrated to a concentration of about 10 times that before the addition of polylysine, whereas the right sample in 6-1) of FIG. 3 depicts a state of being at a concentration comparable to that of the right sample in 5) of FIG. 3 or being diluted according to the addition of NaCl.

In addition, the left sample in 6-2) of FIG. 3 depicts that even when a buffer, water or the like is added to the left sample in 6-1) of FIG. 3 to adjust the amount, the re-dissolved state remains unchanged. The left sample in 6-2) of FIG. 3 depicts a state in which by addition of a buffer to the left sample in 6-1) of FIG. 3, a protein concentration comparable to that before the addition of polylysine is attained.

The left sample in 6-3) of FIG. 3 depicts a state in which NaCl has been added to the left sample in 5) of FIG. 3. The right sample in 6-3) of FIG. 3 is a figure depicting a state in which part of the right sample in 5) of FIG. 3 has been removed so as to obtain an amount comparable to the amount of the left sample in 5) of FIG. 3 and, further, NaCl has been added. The left sample in 6-3) of FIG. 3 is in a re-solubilized state concentrated to a concentration of about 10 times that before the addition of polylysine, in the same manner as the left sample in 6-1) of FIG. 3, whereas the right sample in 6-3) of FIG. 3 represents a state of being diluted according to the addition of NaCl, as compared to the state before the addition of polylysine, and, further, a state in which 90 mass % of the protein has been lost.

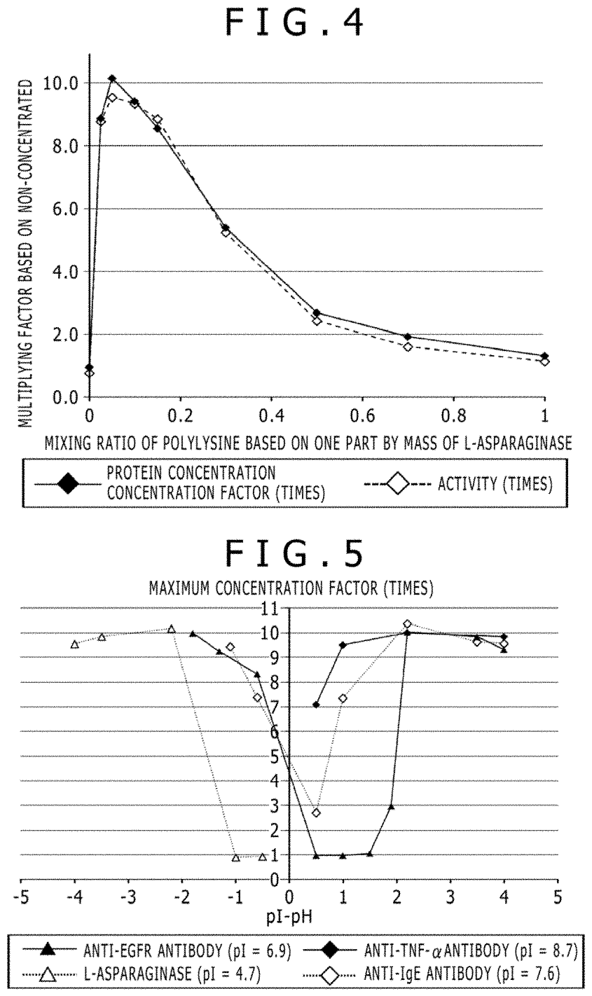

FIG. 4 depicts graphs depicting concentration factor of L-asparaginase in relation to the ratio in mixing 1 part by mass of L-asparaginase and polylysine that are added to a MOPS buffer. The concentration factor of L-asparaginase is obtained by a method in which a complex obtained by centrifugation of a complex-containing aqueous suspension is re-dissolved by addition of NaCl thereto, and, in the aqueous liquid obtained, the L-asparaginase concentration is calculated from absorbance.

The disclosed complex is a complex formed when a protein and a polyamino acid that have surface charge are contained in a buffer and the protein and the polyamino acid interact with each other, preferably on an electrostatic basis. For the complex, a concentration factor in excess of 1 can be obtained when the absolute value of the difference between pH of the buffer and isoelectric point pI of the protein is in the range of preferably from 0.5 to 4.0. As depicted in FIG. 5, independently of the kind of the protein, the concentration factor increases where |pH−pI| ranges from about 0.5 to 2, a concentration factor of about 10 times being obtained, and thereafter a concentration factor remaining on the same level is exhibited irrespectively of the value of |pH−pI|. In other words, it is seen that suitable concentration can be achieved when |pH−pI| is in the range of preferably 0.5 to 4.0, more preferably from 1.0 to 4.0, more preferably from 1.0 to 3.5, more preferably from 1.8 to 3.5, more preferably from 1.8 to 3.0, and further preferably from 1.8 to 2.5.

It has not been precisely determined why the maximum concentration factor of the protein in the complex of the protein and the polyamino acid lies where the absolute value of the difference between the pH of the buffer and the isoelectric point pI of the protein is in the range from 0.5 to 4.0. It has been considered that the fact that the maximum concentration factor of the protein in the complex is present where the absolute value of the difference between the pH of the buffer and the isoelectric point pI of the protein is in the range of preferably from 1.5 to 4.0, more preferably from 1.0 to 4.0, more preferably from 1.0 to 3.5, more preferably from 1.5 to 3.5, more preferably from 1.8 to 3.5, more preferably from 1.8 to 3.0, and further preferably from 1.8 to 2.5 is one characteristic that represents a state of an electrostatic interaction by which the protein and the polyamino acid form the complex, but the present invention is not limited to these mechanisms.

(4) The protein is stable in the complex, and its activity is not deteriorated when it is concentrated.

Here, the statement that the activity is not deteriorated refers to that even when the complex is subjected to a concentrating operation, its biological activity is retained at a level of not less than 80% based on that in a control liquid of the same protein at the same concentration in the same buffer. More preferably, the level is not less than 85%, or not less than 90%. In some cases, the measured value exceeds 100%, and it is not clear whether such a measured value is due to measurement errors or due to some mechanism.

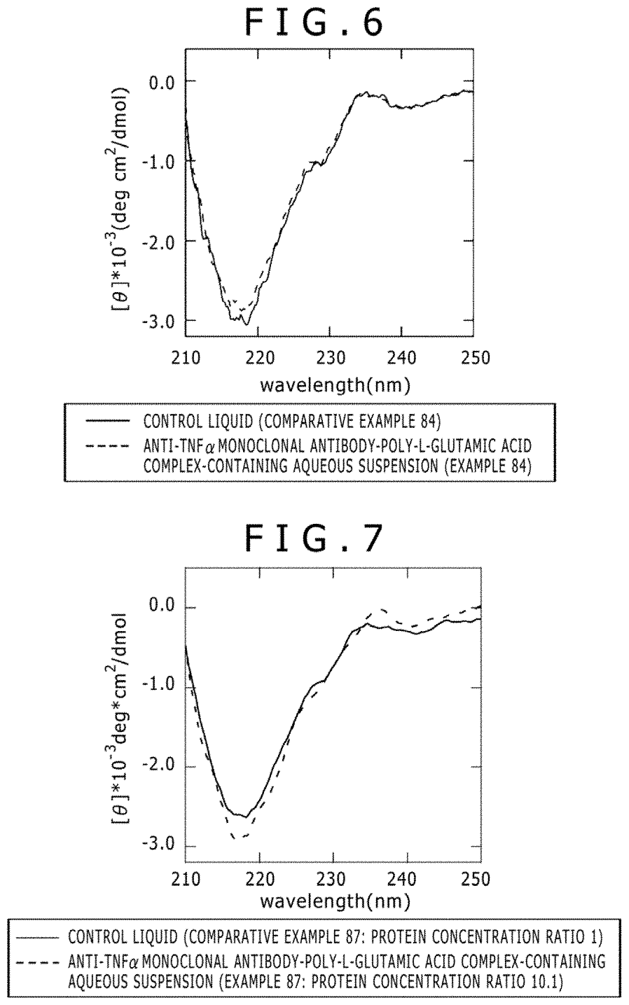

In addition, the protein is stable in the complex, the secondary structure of the protein is retained after the complex is formed and then solubilized, and the secondary structure of the protein is retained even after concentration. It is known that even when a protein forms the disclosed complex and is subjected to concentration and then to re-dissolution, the secondary structure of the protein is little changed. This has been verified by measurement of the CD spectra before formation of the complexes and the CD spectra after the complex formation and re-dissolution, as depicted in FIGS. 6, 7, 8, and 9 which respectively depict the results of Examples 84, 87, 88, and 88-2 to be described later. While the concentration in the concentrated state of the high-purity protein is not limited, it is estimated from the aforesaid experiments that the secondary structure of the protein is not changed when the protein is used at a concentration of preferably from 0.1 mg/mL to 300 mg/mL.

The disclosed protein-polyamino acid complexes can be described as complexes [1] to [5] which are characterized by the following five characteristic properties and have different uses according to their characteristic properties.

### 1. Protein-Polyamino Acid Complex Having Shaking Stress Resistance (Hereinafter Referred to as Complex [1])

The complex [1] has the following characteristic properties (1) to (5).

(1) The complex [1] of a protein and a polyamino acid is obtained in a complex-containing aqueous suspension in which the protein and the polyamino acid form the complex in a buffer and are suspended in the buffer.

(2) The protein can be concentrated by forming the complex [1] in the buffer and removing at least part of water from the complex-containing aqueous suspension obtained.

(3) The complex [1] can be dissolved by adding a low-concentration electrolyte to the complex-containing aqueous suspension.

(4) The activity of the protein is not damaged when the protein is concentrated using the complex. Besides, the secondary structure of the protein is retained after the protein is subjected to complex formation and then solubilization, and the protein retains its secondary structure even when concentrated.

(5) The protein has shaking stress resistance in the complex [1].

The characteristic properties (1) to (4) are the same as described in 1. Protein-Polyamino Acid Complex above, and, therefore, the descriptions thereof are omitted here. The characteristic property (5) will be described below.

(5) The protein has shaking stress resistance in the complex.

Here, shaking stress resistance refers to the complex [1] that has a high shaking stress resistance, specifically, has a high activity retention rate, or retains the structure of the protein better, or has a high protein retention rate, as compared with a control liquid of the same protein at the same concentration in the same buffer.

The shaking stress refers to application of shaking of, for example, 100 rpm to 500 rpm for 10 hours to 100 hours, as indicated in Test Examples in Examples described later, to a protein or complex in a buffer. The shaking stress resistance, which differs according to the kind of protein and hence cannot be restricted, refers to that activity retention rate, or the ratio of protein activity after shaking stress to protein activity before shaking stress, is 10% to 99% higher than that in the case where not any complex is formed. Preferably, the activity retention rate may be 10% to 50% higher, more preferably 15% to 50% higher.

In addition, when the concentration of a protein is measured in terms of absorbance before and after the shaking stress, the following situations are observed. In the case where a complex is not formed, a large amount of insoluble precipitate is formed, so that when aggregates are removed by centrifugation after shaking, the concentration of the protein in the buffer is lowered, and the retention rate of the protein content is lowered, down to below 7% in some cases. On the other hand, in the case where the complex is formed, the protein in the complex is stable before and after the shaking stress, and the retention rate of the protein content may be 100% or higher depending on measurement. The value of retention rate of the content of the protein in the complex is preferably 15% to 99% higher, more preferably 18% to 95% higher, and further preferably 25% to 90% higher, than the value of retention rate of the content of the protein in the control liquid in which not any complex is formed.

### 2. Protein-Polyamino Acid Complex Having Fluidity (Hereinafter Referred to as Complex [2])

The complex [2] the following characteristic properties (1) to (5).

(1) The complex [2] of a protein and a polyamino acid is obtained in a complex-containing aqueous suspension in which the protein and the polyamino acid form the complex in a buffer and are suspended in the buffer.

(2) The protein can be concentrated by forming the complex [2] in the buffer and removing at least part of water from the complex-containing aqueous suspension obtained.

(3) The complex [2] can be dissolved by adding a low-concentration electrolyte to the complex-containing aqueous suspension.

(4) The activity of the protein is not damaged when the protein is concentrated using the disclosed complex [2]. Besides, the secondary structure of the protein is retained after the protein is subjected to complex formation and then solubilization, and the protein retains its secondary structure even when concentrated.

(5) The protein is excellent in fluidity in the complex [2].

The characteristic properties (1) to (4) are the same as described in 1. Protein-Polyamino Acid Complex above, and, therefore, the descriptions thereof are omitted here. The characteristic property (5) will be described below.

(5) The protein is excellent in fluidity in the complex [2].

Here, to have fluidity, to be excellent in fluidity, or fluidity enhancement refers to the complex [2] that is more fluid than a control liquid of the same protein at the same concentration in the same buffer; specifically, it refers to that the viscosity of the protein contained in the complex [2] is low, as compared at the same concentration in the same buffer.

The viscosity of the protein in the complex [2] differs depending on the kind and concentration of the protein, and is not limited. The viscosity may be not more than 60%, preferably not more than 55%, and further preferably not more than 50%, based on the viscosity of the control liquid.

### 3. Protein-Polyamino Acid Complex Having Oxidation Resistance (Hereinafter Referred to as Complex [3])

The complex [3] has the following characteristic properties (1) to (5).

(1) The complex [3] of a protein and a polyamino acid is obtained in a complex-containing aqueous suspension in which the protein and the polyamino acid form the complex in a buffer and are suspended in the buffer.

(2) The protein can be concentrated by forming the complex [3] in the buffer and removing at least part of water from the complex-containing aqueous suspension obtained.

(3) The complex [3] can be dissolved by adding a low-concentration electrolyte to the complex-containing aqueous suspension.

(4) The activity of the protein is not damaged when the protein is concentrated using the disclosed complex [3]. Besides, the secondary structure of the protein is retained after the protein is subjected to complex formation and then solubilization, and the protein retains its secondary structure even when concentrated.

(5) The protein has oxidation resistance in the complex [3].

The characteristic properties (1) to (4) are the same as described in 1. Protein-Polyamino Acid Complex above, and, therefore, the descriptions thereof are omitted here. The characteristic property (5) will be described below.

(5) The protein has oxidation resistance in the complex [3].

Here, oxidation stress refers to a change generated in the primary structure of a protein as a result of a process in which an active oxygen species or the like in an oxidizing compound brings about fragmentation, association, modification or the like in an amino acid side chain or chains susceptible to oxidation in the protein. To have oxidation resistance refers to the complex [3] that is higher in oxidation resistance than a control liquid of the same protein at the same concentration in the same buffer, specifically that the activity retention rate of the protein in the complex [3] in response to addition of an oxidizing compound is high, or the structure of the protein is retained, as compared to a control liquid to which the same compound is added, at the same concentration in the same buffer.

As an example of the oxidizing compound to be used, there can be mentioned a mixture of 0.01% by weight to 3.00% by weight aqueous hydrogen peroxide and a buffer, 1 mM to 10 mM ascorbic acid in the presence of 1 mM to 10 mM of copper ions, and the like. A change in the activity of the protein or a change in the primary structure of the protein is measured, after the system is kept for a predetermined time at a predetermined temperature. The predetermined time and the predetermined temperature may be, for example, one hour to 10 hours and 20° C. to 40° C.

The oxidation resistance of the protein in the complex [3] differs depending on the kind and concentration of the protein and is therefore not restricted. As for the difference from the retained activity of a control liquid under the same oxidation stress, the retained activity of the protein in the complex [3] may be 5% to 30% higher, preferably 5% to 25% higher, and further preferably 10% to 20% higher.

Besides, as for retention of the structure of the protein, let the change rate of primary structure in a control liquid (Comparative Example) in which a complex is not formed, under the same oxidation stress, be 100%, then the change rate of primary structure of the protein in the complex is suppressed to preferably 95% or below, more preferably 91% or below, and further preferably 87% or below.

4. Protein-Polyamino Acid Complex Having Thermal Resistance (Hereinafter Referred to as Complex [4])

The complex [4] has the following characteristic properties (1) to (5).

(1) The complex [4] of a protein and a polyamino acid is obtained in a complex-containing aqueous suspension in which the protein and the polyamino acid form the complex in a buffer to be suspended in the buffer.

(2) The protein can be concentrated by forming the complex [4] in the buffer and removing at least part of water from the complex-containing aqueous suspension obtained.

(3) The complex [4] can be dissolved by adding a low-concentration electrolyte to the complex-containing aqueous suspension.

(4) The activity of the protein is not damaged when the protein is concentrated using the disclosed complex [4]. Besides, the secondary structure of the protein is retained after the protein is subjected to complex formation and then solubilization, and the protein retains its secondary structure even when concentrated.

(5) The protein has thermal resistance in the complex [4].

The characteristic properties (1) to (4) are the same as described in 1. Protein-Polyamino Acid Complex above, and, therefore, the descriptions thereof are omitted here. The characteristic property (5) will be described below.

Here, to have thermal resistance refers to the complex [4] that is higher in thermal resistance than a control liquid of the same protein at the same concentration in the same buffer, specifically that the activity of the protein in the complex [4] after thermal load is high, or the denaturing temperature of the protein is high, as compared to a control liquid in which not any complex is formed, at the same concentration in the same buffer.

The thermal load refers to an application of heat to the complex [4] or to a control liquid of the same protein at the same concentration in the same buffer, at a predetermined temperature for a predetermined time, for example, at room temperature to 60° C. for five minutes to 20 hours. The thermal resistance can be evaluated by a method in which the activity in the case where the complex [4] or a control liquid of the same protein at the same concentration in the same buffer is stored in a cold place is assumed to be 100%, and the activity retention rate in the complex [4] after the aforesaid thermal load or the control liquid of the same protein at the same concentration in the same buffer is measured. Alternatively, the thermal resistance can be evaluated by a method in which the complex [4] and the control liquid of the same protein at the same concentration in the same buffer are put to differential scanning calorimetry to measure the respective denaturing temperatures.

The thermal resistance of the protein in the complex [4] differs depending on the kind and concentration of the protein, and is not restricted. The difference from the activity detention rate in the control liquid under the same thermal stress may be 5% to 95%, preferably 10% to not more than 95%, and further preferably 20% to 85%.

The thermal denaturing temperature of the protein in the complex [4] is preferably 1° C. to 20° C. high, as compared to the control liquid. 5. Protein-polyamino acid complex having aggregation inhibitory properties (hereinafter referred to as complex [5])

The complex [5] has the following characteristic properties (1) to (5).

(1) The complex [5] of a protein and a polyamino acid is obtained in a complex-containing aqueous suspension in which the protein and the polyamino acid form the complex in a buffer to be suspended in the buffer.

(2) The protein can be concentrated by forming the complex [5] in the buffer and removing at least part of water from the complex-containing aqueous suspension obtained.

(3) The complex [5] can be dissolved by adding a low-concentration electrolyte to the complex-containing aqueous suspension.

(4) The activity of the protein is not damaged when the protein is concentrated using the disclosed complex [5]. Besides, the secondary structure of the protein is retained after the protein is subjected to complex formation and then solubilization, and the protein retains its secondary structure even when concentrated.

(5) The protein has aggregation inhibitory properties in the complex [5].

The characteristic properties (1) to (4) are the same as described in 1. Protein-Polyamino Acid Complex above, and, therefore, the descriptions thereof are omitted here. The characteristic property (5) will be described below.

Here, to have aggregation inhibitory properties refers to the complex [5] that is superior in aggregation inhibition to a control liquid of the same protein at the same concentration in the same buffer, and specifically that the formation of aggregates is restrained by the formation of the complex. The formation of aggregates can be evaluated by a method in which aggregation amounts of protein before and after shaking stress in the complex [5] and in a control liquid of the same protein at the same concentration in the same buffer are calculated by subtracting the mass of protein in the supernatant from the total mass of protein, or a method in which soluble aggregate peak area according to size exclusion chromatography is measured. Alternatively, the formation of aggregates can be evaluated by calculating the increase rate of the number of aggregates of protein or the increase rate of turbidity, before and after thermal stress, for the complex [5] and a control liquid of the same protein at the same concentration in the same buffer. By these measurements, it can be evaluated that the protein in the complex [5] is restrained from the formation of aggregates under various conditions.

The aggregation inhibitory properties of the protein in the complex [5] differ depending on the kind and concentration of the protein, and are not restricted. The complex [5] may have such a characteristic property that the difference in insoluble aggregate formation rate from the control liquid under the same shaking stress is 5% to 20%, preferably that aggregates are substantially absent after the shaking stress. The increase rate of soluble aggregates under the same shaking stress, in terms of ratio to that of the control liquid, may be 30% or below, preferably 10% or below, and further preferably 5% or below. The increase rate of the number of aggregates under the same shaking stress, in terms of ratio to that of the control liquid, may be 1.0% or below, preferably 0.5% or below, and further preferably 0.2% or below. The increase rate of turbidity under the same thermal stress, in terms of ratio to that of the control liquid, may be 10% or below, preferably 6% or below, and further preferably 4% or below.

Characteristic properties of the aqueous suspension preparation containing the protein-polyamino acid complex

The protein has a positive or negative surface charge. The polyamino acid is cationic or anionic.

The molecular weight of the cationic, neutral, or anionic polyamino acid is preferably in the range from 0.5 kDa to 1000 kDa. More preferably, the molecular weight is 5 kDa to 800 kDa, and further preferably 10 kDa to 500 kDa. The molecular weight of the protein is preferably in the range from 3 kDa to 670 kDa. The 50% cell growth inhibitory concentration of the polyamino acid is preferably not less than 0.2%, more preferably not less than 1.0%.

In the case where the protein has a positive surface charge, the protein is preferably combined with an anionic polyamino acid. In the case where the protein has a negative surface charge, the protein is preferably combined with a cationic polyamino acid.

The protein is contained in the aqueous suspension at a concentration in the range from 0.01 mg/mL to 500 mg/mL.

The pH of the buffer used for the aqueous suspension is preferably in the range from pH 3 to pH 10.5.

The disclosed aqueous suspension preparation is a stable protein-polyamino acid complex-containing aqueous suspension preparation which contains a protein and a polyamino acid that have surface charge in a buffer, wherein the blending ratio of these ingredients is preferably such as to contain 0.005 parts by mass to 6 parts by mass of the polyamino acid based on 1 part by mass of the protein, the pH of the buffer is adjusted to be deviated from the isoelectric point pI of the protein contained therein to the basic side or acidic side by not less than 0.5, and the protein and the polyamino acid form a complex by electrostatic interaction.

The disclosed aqueous suspension preparation can be concentrated and stabilized easily by only selecting the pH of the buffer and the polyamino acid according to the isoelectric point of the protein, and does not need addition of an additive or additives, which has been necessary conventionally. An additive or additives other than the buffer, protein, polyamino acid, and pH adjuster can be added to the aqueous suspension preparation, but it is unnecessary to add such additives. Besides, the aqueous suspension preparation does not need an intricate dissolving operation required for a freeze-dried pharmaceutical preparation, and can be administered as it is as a pharmaceutical preparation. Alternatively, the aqueous suspension preparation can be administered as a pharmaceutical preparation after adding an inorganic salt represented by sodium chloride thereto and dissolving it.

Combination of protein and polyamino acid for forming protein-polyamino acid complex

The disclosed complex contains a protein and an ionic polyamino acid.

1. (1) In the case of a protein (enzyme, cytokine, antibody fragment, or the like) having an isoelectric point in an acidic region (pH not less than 3.0 and less than 6.0), in the case of using a buffer having a buffer capacity on the acidic side of the isoelectric point, at least one anionic polyamino acid selected from the group consisting of polyglutamic acid (MW: 750 to 5000, pI: 2.81 to 3.46), polyglutamic acid (MW: 3000 to 15000, pI: 2.36 to 3.00), polyglutamic acid (MW: 15000 to 50000, pI: 1.85 to 2.36), polyglutamic acid (MW: 50000 to 100000, pI: 1.56 to 1.85), polyaspartic acid (MW: 2000 to 11000, pI: 2.06 to 2.75), polyaspartic acid (MW: 5000 to 15000, pI: 1.93 to 2.39) and their water-soluble salts is added, to form a complex. Besides, in the case of using a buffer having a buffer capacity on the basic side of the isoelectric point, at least one cationic polyamino acid selected from the group consisting of polylysine (MW: 1000 to 5000, pI: 10.85 to 11.58), polylysine (MW: 4000 to 15000, pI: 11.49 to 12.06), polylysine (MW: 15000 to 30000, pI: 12.06 to 12.37), polylysine (MW: 30000 or above, pI: 12.37 or above), polyarginine (MW: 5000 to 15000, pI: 13.49 to 13.97), polyarginine (MW: 15000 to 70000, pI: 13.98 to 14.00), polyarginine (MW: 70000 or above, pI: 14.00), polyhistidine (MW: 5000 to 25000, pI: 7.74 to 8.30) and their water-soluble salts is added, to form a complex.

(2) Note that as the buffer for the protein having the isoelectric point in an acidic region, known buffers can be used appropriately, without any particular restrictions. As the buffer, there may be used, for example, phosphate buffer, citrate buffer, citrate-phosphate buffer, tartarate buffer, trishydroxymethylaminomethane-HCl buffer (tris-hydrochloric acid buffer), MES buffer (2-morpholinoethanesulfonic acid buffer), TES buffer (N-tris(hydroxymethyl)methyl-2-aminoethanesulfonic acid buffer), acetate buffer, MOPS buffer (3-morpholinopropanesulfonic acid buffer), MOPS-NaOH buffer, HEPES buffer (4-(2-hydroxyethyl)-1-piperazineethanesulfonic acid buffer), HEPES-NaOH buffer, PIPES buffer (piperazine-1,4-bis(2-ethanesulfonic acid) buffer), and the like GOOD buffers, glycine-hydrochloric acid buffer, glycine-NaOH buffer, glycylglycine-NaOH buffer, glycylglycine-KOH buffer and the like amino acid buffers, Tris-boric acid buffer, boric acid-NaOH buffer, borate buffer and the like boric acid buffers, or imidazole buffer, etc.

2. (1) In the case of a protein (antibody, antibody fragment, fusion protein, or the like) having an isoelectric point in a neutral region (pH 6.0 to 8.0), in the case of using a buffer having a buffer capacity on the acidic side of the isoelectric point, at least one anionic polyamino acid selected from the group consisting of polyglutamic acid (MW: 750 to 5000, pI: 2.81 to 3.46), polyglutamic acid (MW: 3000 to 15000, pI: 2.36 to 3.00), polyglutamic acid (MW: 15000 to 50000, pI: 1.85 to 2.36), polyglutamic acid (MW: 50000 to 100000, pI: 1.56 to 1.85), polyaspartic acid (MW: 2000 to 11000, pI: 2.06 to 2.75), polyaspartic acid (MW: 5000 to 15000, pI: 1.93 to 2.39) and their water-soluble salts is added, to form a complex. Besides, in the case of using a buffer having a buffer capacity on the basic side of the isoelectric point, at least one cationic polyamino acid selected from the group consisting of polylysine (MW: 1000 to 5000, pI: 10.85 to 11.58), polylysine (MW: 4000 to 15000, pI: 11.49 to 12.06), polylysine (MW: 15000 to 30000, pI: 12.06 to 12.37), polylysine (MW: 30000 or above, pI: 12.37 or above), polyarginine (MW: 5000 to 15000, pI: 13.49 to 13.97), polyarginine (MW: 15000 to 70000, pI: 13.98 to 14.00), polyarginine (MW: 70000 or above, pI: 14.00), polyhistidine (MW: 5000 to 25000, pI: 7.74 to 8.30) and their water-soluble salts is added, to form a complex.

(2) Note that as the buffer for the protein having the isoelectric point in a neutral region, known buffers can be used appropriately, without any particular restrictions. As the buffer, there may be used, for example, phosphate buffer, citrate buffer, citrate-phosphate buffer, tartarate buffer, trishydroxymethylaminomethane-HCl buffer (Tris-hydrochloric acid buffer), MES buffer (2-morpholinoethanesulfonic acid buffer), TES buffer (N-tris(hydroxymethyl)methyl-2-aminoethanesulfonic acid buffer), acetate buffer, MOPS buffer (3-morpholinopropanesulfonic acid buffer), MOPS-NaOH buffer, HEPES buffer (4-(2-hydroxyethyl)-1-piperazineethanesulfonic acid buffer), HEPES-NaOH buffer, PIPES buffer (piperazine-1,4-bis(2-ethanesulfonic acid) buffer), and the like GOOD buffers, glycine-hydrochloric acid buffer, glycine-NaOH buffer, glycylglycine-NaOH buffer, glycylglycine-KOH buffer and the like amino acid buffers, Tris-boric acid buffer, boric acid-NaOH buffer, borate buffer and the like boric acid buffers, or imidazole buffer, etc.

3. (1) In the case of a protein (cytokine, hormone, antibody, antibody fragment, or the like) having an isoelectric point in a basic region (pH more than 8.0 and not more than 11.0), in the case of using a buffer having buffer capacity on the acidic side of the isoelectric point, at least one anionic polyamino acid selected from the group consisting of polyglutamic acid (MW: 750 to 5000, pI: 2.81 to 3.46), polyglutamic acid (MW: 3000 to 15000, pI: 2.36 to 3.00), polyglutamic acid (MW: 15000 to 50000, pI: 1.85 to 2.36), polyglutamic acid (MW: 50000 to 100000, pI: 1.56 to 1.85), polyaspartic acid (MW: 2000 to 11000, pI: 2.06 to 2.75), polyaspartic acid (MW: 5000 to 15000, pI: 1.93 to 2.39) and their water-soluble salts is added, to form a complex. Besides, in the case of using a buffer having a buffer capacity on the basic side of the isoelectric point, at least one cationic polyamino acid selected from the group consisting of polylysine (MW: 1000 to 5000, pI: 10.85 to 11.58), polylysine (MW: 4000 to 15000, pI: 11.49 to 12.06), polylysine (MW: 15000 to 30000, pI: 12.06 to 12.37), polylysine (MW: 30000 or above, pI: 12.37 or above), polyarginine (MW: 5000 to 15000, pI: 13.49 to 13.97), polyarginine (MW: 15000 to 70000, pI: 13.98 to 14.00), polyarginine (MW: 70000 or above, pI: 14.00), polyhistidine (MW: 5000 to 25000, pI: 7.74 to 8.30) and their water-soluble salts is added, to form a complex.

(2) Note that as the buffer for the protein having the isoelectric point in a basic region, known buffers can be used appropriately, without any particular restrictions. As the buffer, there may be used, for example, phosphate buffer, citrate buffer, citrate-phosphate buffer, tartarate buffer, trishydroxymethylaminomethane-HCl buffer (Tris-hydrochloric acid buffer), MES buffer (2-morpholinoethanesulfonic acid buffer), TES buffer (N-tris(hydroxymethyl)methyl-2-aminoethanesulfonic acid buffer), acetate buffer, MOPS buffer (3-morpholinopropanesulfonic acid buffer), MOPS-NaOH buffer, HEPES buffer (4-(2-hydroxyethyl)-1-piperazineethanesulfonic acid buffer), HEPES-NaOH buffer, PIPES buffer (piperazine-1,4-bis(2-ethanesulfonic acid) buffer), and the like GOOD buffers, glycine-hydrochloric acid buffer, glycine-NaOH buffer, glycylglycine-NaOH buffer, glycylglycine-KOH buffer and the like amino acid buffers, Tris-boric acid buffer, boric acid-NaOH buffer, borate buffer and the like boric acid buffers, or imidazole buffer, etc.

Method of preparing the aqueous suspension preparation containing the protein-polyamino acid complex

(Step 1): The pH of a buffer is adjusted so that the pH is deviated to the basic side and/or the acidic side from the isoelectric point pI of the protein by an absolute value of 0.5 to 4.0.

(Step 2): To the buffer, the protein having a surface charge is added so as to attain a concentration in the range from 0.01 mg/mL to 50 mg/mL, and the polyamino acid is added so as to attain a concentration in the range from 0.01 mg/mL to 100 mg/mL, thereby forming an aqueous suspension which contains the protein-polyamino acid complex.

(Step 3): At least part of water or buffer is removed from the aqueous suspension containing the protein-polyamino acid complex by a method selected from among centrifugation, ultrafiltration, supernatant removal and the like, thereby making an adjustment such that the protein is contained at a concentration in the range from 0.1 mg/mL to 500 mg/mL.

(Step 4): To the aqueous suspension prepared in the step 3, a buffer or water or the like in an amount smaller than the amount of the water or buffer removed in the step 3 is added, to thereby obtain an aqueous suspension containing the protein-polyamino acid complex having a concentrated protein concentration. Alternatively, a buffer or water or the like in an amount equal to the amount of the water or buffer removed in the step 3 is added, to thereby obtain an aqueous suspension containing the protein-polyamino acid complex having an equal protein concentration. Yet alternatively, a buffer or water or the like in an amount larger than the amount of the water or buffer removed in the step 3 is added, thereby to obtain an aqueous suspension containing the protein-polyamino acid complex having a diluted protein concentration.

(Step 5): Without removing water from the complex-containing aqueous suspension obtained after the above step 1 and step 2, a buffer or water or the like is added to the aqueous suspension, to thereby obtain an aqueous suspension containing the protein-polyamino acid complex having a diluted protein concentration.

In the case where the protein is a protein for medical use, the aqueous suspension preparation is produced by the step 1, step 2, step 3, and step 4, or by the step 1, step 2, and step 5.

The disclosed pharmaceutical preparation is an aqueous suspension protein preparation in which a protein preparation and a polyamino acid that have surface charge in a buffer form a complex to be suspended in the buffer, wherein the protein preparation can be concentrated by removing at least part of water from the complex-containing aqueous suspension preparation, and the protein preparation is stabilized in the complex. In addition, a method of stabilizing a medical protein is provided by causing a protein preparation and a polyamino acid that have surface charge in a buffer to form a complex in the buffer, thereby obtaining an aqueous suspension preparation. The concentration of the complex in the aqueous suspension preparation may be enhanced, or the complex may be caused to gather to form a layer separated from a small amount or very small amount of water layer, resulting in a two-layer state. Besides, the complex may be separated as in a wet state. Further, a low-concentration electrolyte as an inorganic salt may be added to the complex in the aqueous suspension preparation, to allow dissolution, thereby obtaining an aqueous preparation and putting it to medical use. In the case where the pH upon dissociation of the aqueous suspension preparation by use of a salt is highly basic, for example pH 8 or above, or highly acidic, for example pH 4 or below, which is unsuitable for administration into a human, an acidic buffer or a basic buffer may be added, whereby the pH can be adjusted to the vicinity of a neutral point suitable for administration.

The protein preparation for medical use is not restricted, and is at least one of enzyme, cytokine, hormone, antibody, antibody fragment, and fusion protein for medical use.

Specific examples of the protein preparation for medical use include the following, but the protein preparations are not restricted to these examples.

### (Enzymes)

alteplase, monteplase, imiglucerase, velaglucerase alfa, agalsidase alfa, agalsidase beta, laronidase, alglucosidase alfa, idursulfase, galsulfase, rasburicase, dornase alfa, asparaginase, PEG-asparaginase, condoliase

### (Blood Coagulation Fibrinolysis System Factor)

batroxobin, octocog alfa, rurioctocog alfa, eptacog alfa, efraloctocog alfa, turoctocog alfa, eftrenonacog alfa, blood clotting factor,

thrombomodulin alfa

### (Serum Protein)

serum albumin,

### (Hormones)

human insulin, insulin lispro, insulin aspart, insulin glargine, insulin detemir, insulin glulisine, insulin degludec, liraglutide, somatropin, pegvisomant, mecasermin, carperitide, glucagon, follitropin alfa, follitropin beta, teriparatide, metreleptin,

### (Vaccines)

recombinant adsorbed Hepatitis B vaccine, dry cell culture inactivated Hepatitis A vaccine, recombinant adsorbed bivalent human papillomavirus-like particle vaccine, recombinant adsorbed quadrivalent human papillomavirus-like particle vaccine,

### (Interferons)

interferon alfa, albumin-modified interferon alfa, interferon alfa-2b, interferon alfacon, interferon beta, interferon beta-1a, interferon beta-2b, interferon gamma-1a, peginterferon alfa-2a, peginterferon alfa-2b,

### (Erythropoietins)

epoetin alfa, epoetin beta, darbepoetin alfa, epoetin beta pegol, epoetin kappa,

### (Cytokines)

filgrastim, pegfilgrastim, lenograstim, nartograstim, celmoleukin, teceleukin, trafermin,

### (Antibodies)

muromonab-CD3, trastuzumab, rituximab, palivizumab, infliximab, basiliximab, tocilizumab, gemtuzumab ozogamicin, bevacizumab, ibritumomab tiuxetan, adalimumab, cetuximab, ranibizumab, omalizumab, eculizumab, panitumumab, ustekinumab, golimumab, canakinumab, denosumab, mogamulizumab, certolizumab prgol, ofatumumab, pertuzumab, trastuzumab emtansine, brentuximab vedotin, natalizumab, nivolumab, alemtuzumab, iodine 131-modified tositumomab, catumaxomab, adecatumumab, edrecolomab, abciximab, siltuximab, daclizumab, efalizumab, obinutuzumab, vedolizumab, pembrolizumab, ixekizumab, diridavumab, ipilimumab, belimumab, raxibacumab, ramucirumab,

### (Fusion Proteins)

etanercept, abatacept, romiplostim, aflibercept.

The disclosed aqueous suspension preparation or aqueous preparation does not have general toxicity, as will be depicted later. The aqueous suspension preparation or aqueous preparation is turned into pharmaceutical preparations for arbitrary routes of administration including parenteral, subcutaneous, intraperitoneal, intrapulmonary, and intranasal administrations, and, if necessary, for local therapy and intralesional administration. As the parenteral infusion, there may be mentioned intravenous, intraarterial, intraperitoneal, intramuscular, intracutaneous, or subcutaneous administration. The preparation is preferably administered by injection, and most preferably by intravenous or subcutaneous injection. The preferable administration includes local administration, particularly, percutaneous, transmucosal, rectum, and peroral administrations, or local administration by way of a catheter placed near a desired part, for example. The preparation may contain a pharmaceutically permissible excipient or diluent, according to the route of administration. As examples of such an excipient, there may be mentioned water, pharmaceutically allowable organic solvents, etc.

For the disclosed aqueous suspension preparation or aqueous preparation, an administrating device such as a prefilled syringe or a two-air-chamber prefilled syringe may be mentioned as an example. In the case of administering a protein for medical use, conventionally, intravenous drip, subcutaneous injection or the like has been performed, and administration by health care staff in a medical institution or self-administration at the patient's home has been conducted. In the case where the protein for medical use is insufficient in thermal stability, cold storage may be needed. Storing prefilled syringes for self-injection or the like in a refrigerator is a heavy burden on the patient. In the case where a syringe having two air chambers, for example, is used while holding a complex-containing aqueous suspension preparation having thermal stability as depicted in Examples 102 to 106 described later in one of the chambers, and holding a low-concentration electrolyte in the other chamber, to thereby enhance shelf stability, it is considered possible to store the syringe at room temperature instead of cold storage. This, if realized, has a great merit. In using the syringe, the two air chambers are brought into fluid communication with each other, whereby the preparation can be administered as an aqueous preparation.

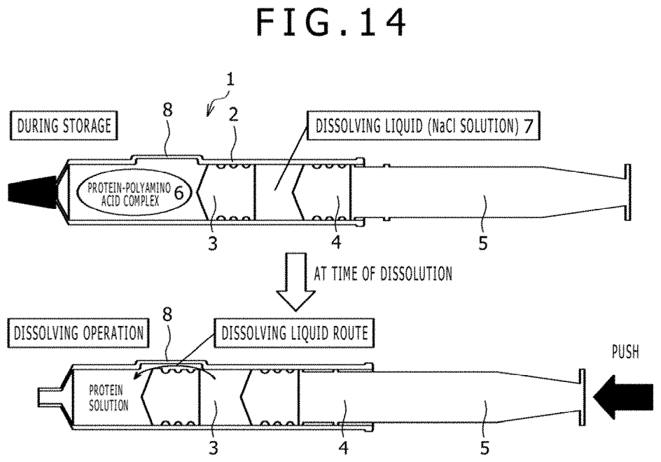

FIG. 14 depicts an example of the two-air-chamber prefilled syringe. A prefilled syringe 1 includes a sheath 2, gaskets 3 and 4 slidable within the sheath 2, and a plunger 5 (proximal side) for operation to move the gaskets 3 and 4. In a first chamber on the distal side which is surrounded by the sheath 2 and the gasket 3, a protein-polyamino acid complex 6 is preliminarily accommodated in a liquid-tight manner. In a second chamber on the proximal side which is separated from the first chamber by the gasket 3, an aqueous electrolyte solution as a dissolving liquid 7 is accommodated and is sealed by the plunger 5 and the gaskets 3 and 4. The sheath 2 is provided with an enlarged diameter portion 8 at the first chamber. This state exists during storage. During storage, a protein for medical use is preserved in the disclosed polyamino acid complex with high stability.

At the time of dissolution, the plunger 5 is pushed to make the gasket 4 push the dissolving liquid 7, so that the gasket 3 pushed by the dissolving liquid 7 moves to the distal side where the enlarged diameter portion 8 exists, the dissolving liquid 7 flows into the first chamber on the distal side by way of the enlarged diameter portion 8, and is mixed with the protein-polyamino acid complex 6, to re-dissolve the protein in the complex, thereby forming a protein solution.

Method of concentrating protein, method of producing aqueous preparation having concentrated, or equivalent, or diluted protein concentration

(Step 1): The pH of a buffer is adjusted so that the pH is deviated to the basic side and/or the acidic side from the isoelectric point pI of the protein by an absolute value of, for example, 0.5 to 4.0.

(Step 2): To the buffer, the protein having a surface charge is added so as to attain a concentration in the range from 0.01 mg/mL to 50 mg/mL, and the polyamino acid is added so as to attain a concentration in the range from 0.01 mg/mL to 100 mg/mL, thereby forming an aqueous suspension which contains the protein-polyamino acid complex.

(Step 3): At least part of water or buffer is removed from the complex-containing aqueous suspension containing obtained after the step 1 and step 2, by a method selected from among centrifugation, ultrafiltration, supernatant removal and the like, thereby adjusting the aqueous suspension so that the protein is contained at a concentration in the range from 0.1 mg/mL to 500 mg/mL.

(Step 4): To the aqueous suspension prepared in the step 3, a low-concentration electrolyte in an amount smaller than the amount of water or buffer removed in the step 3 is added, so as to re-dissolve the protein in the complex into the buffer, thereby obtaining an aqueous liquid having a concentrated protein concentration. Alternatively, a low-concentration electrolyte in an amount equal to the amount of water or buffer removed in the step 3 is added, so as to re-dissolve the protein in the complex, thereby obtaining an aqueous liquid having an equivalent protein concentration. Yet alternatively, a low-concentration electrolyte in an amount larger than the amount of water or buffer removed in the step 3 is added, so as to re-dissolve the protein in the complex, thereby obtaining an aqueous liquid having a diluted protein concentration.

(Step 5): Without removing water from the complex-containing aqueous suspension obtained after the step 1 and step 2, a low-concentration electrolyte is added to the aqueous suspension, so as to re-dissolve the protein in the complex into the buffer, thereby obtaining an aqueous liquid having a diluted protein concentration.

In the case where the above-mentioned protein is a protein for medical use, an aqueous preparation is produced by the above step 1, step 2, step 3, and step 4, or by the above step 1, step 2, and step 5.

In the step 1, it is preferable to obtain the complex by adjusting the pH of the buffer so that |pI−pH| will be within the range from 0.5 to 4.0, where pI is the isoelectric point of the protein contained.

### (1) Method of Concentrating Protein

The following steps 1) to 3) can be conducted in an arbitrary order, and may be carried out simultaneously or sequentially.

1) A buffer is added to a protein.
2) A polyamino acid is added to the buffer.
3) The pH of the buffer is adjusted so that |pI−pH| will be not less than 0.5, where pI is the isoelectric point of the protein contained.
4) The protein and the polyamino acid form a complex in the buffer, whereby an aqueous suspension is obtained.
5) At least part of water or buffer is removed. A protein is obtained such that the concentration of the protein in the complex obtained is concentrated as compared to the concentration of the protein in the buffer in the step 1).
6) If necessary, the aqueous suspension obtained is centrifuged, to obtain the complex.

### (2) Method of Dissolving Complex

The complex obtained can be re-dissolved in the aqueous suspension by addition of a low-concentration electrolyte. As examples of the electrolyte, there can be mentioned NaCl, KCl, CaCl₂, MgCl₂ and the like. Preferably, NaCl, which is the highest in biocompatibility, is selected, its concentration being not more than 5% by weight. The concentration of NaCl used for re-dissolution is preferably not less than 8 mM (0.048% by weight), more preferably not less than 40 mM (0.24% by weight).

In order to re-dissolve the obtained complex so as to obtain the protein dissolved in the buffer in a reversible amount, it is preferable to bring the |pI−pH| into the range from 0.5 to 4.0.

### (3) Protein Concentration Measuring Method

The concentration of the protein in the buffer is calculated, for example, from a standard curve obtained by measuring absorbance at a wavelength of 280 nm. The concentration of the protein in the complex is calculated, for example, from a standard curve obtained by measuring absorbance at a wavelength of 280 nm after the complex obtained by centrifugation is dissolved by the method of (2) above. For instance, variation in concentration when the protein is concentrated is calculated in terms of concentration factor based on the non-concentrated. Besides, stress such as shaking, oxidation, heat or the like is loaded on the complex, and variation in concentration after the loading is calculated in terms of retention rate based on the concentration before the loading.

### (4) Method of Measuring Variation in Secondary Structure of Protein

The CD spectrum of the protein in the complex is measured for an aqueous solution obtained by dissolving the protein by the method of (2) above. Stress such as shaking, oxidation, heat or the like is loaded on the complex, the CD spectrum is measured before the loading and after the loading, and variation in the secondary structure of the protein is detected. Similarly, for a control liquid of the protein, the CD spectrum is measured at the same concentration in the same buffer. If comparison of this spectrum with the CD spectrum after the loading of the complex does not depict any variation, it is depicted that the secondary structure of the protein in the complex has not been changed by the aforesaid loading.

### (5) Method of Measuring Activity of Protein

### 1) Activity of L-Asparaginase

Measured in an aqueous solution. The complex is dissolved by the method of (2) above, and measurement is conducted. In relation to a reaction of dissociating asparagine into aspartic acid and ammonia that is catalyzed by L-asparaginase, the concentration of ammonia in the reaction mixture is determined, and the activity of L-asparaginase is determined. For instance, variation in the activity when the protein is concentrated is calculated in terms of multiplying factor of activity based on the activity when the protein is non-concentrated. Besides, stress such as shaking, oxidation, heat or the like is loaded on the complex, and variation in the activity after the loading is calculated in terms of retention rate based on the value before the loading.

### 2) Anti-IgE Monoclonal Antibody Activity (IgE Binding Capacity or IgE Receptor Inhibitory Capacity)

Measured in an aqueous solution. The complex is dissolved by the method of (2) above, and measurement is conducted. In regard of an anti-IgE monoclonal antibody, an activity for binding to IgE or an inhibitory activity on binding of IgE to an IgE receptor is measured by an ELISA method. Both of the activity measurements are assays for examining the binding between IgE and the anti-IgE antibody. In an IgE binding test, the concentration of the anti-IgE monoclonal antibody bound to IgE is measured. In an IgE receptor inhibitory test, the concentration of IgE bound to the IgE receptor is measured. For instance, variation in activity when the protein is concentrated is calculated in terms of multiplying factor of activity based on the activity when the protein is non-concentrated. Besides, stress such as shaking, oxidation, heat or the like is loaded on the complex, and variation in activity after the loading is calculated in terms of retention rate based on the value before the loading.

Specifically, Test Example 4 in Example 74 is measured by an IgE binding test, and activities of other anti-IgE monoclonal antibodies are measured by a receptor inhibition test.

### 3) Anti-TNFα Monoclonal Antibody Activity (TNFα Binding Capacity)

Measured in an aqueous solution. The complex is dissolved by the method of (2) above, and measurement is conducted. In regard of an anti-TNFα monoclonal antibody, activity for binding to TNFα is measured by an ELISA method. For example, variation in activity when the protein is concentrated is calculated in terms of multiplying factor of activity based on the activity when the protein is non-concentrated. Besides, stress such as shaking, oxidation, heat or the like is loaded on the complex, and variation in activity after the loading is calculated in terms of retention rate based on the value before the loading.

### 4) Anti-TNFα Monoclonal Antibody Fab Fragment Activity (TNFα Binding Inhibitory Capacity)

Measured in an aqueous solution. The complex is dissolved by the method of (2) above, and measurement is conducted. In regard of an anti-TNFα monoclonal antibody Fab fragment, inhibitory activity on binding between an anti-TNFα monoclonal antibody and TNFα is measured by an ELISA method. For instance, variation in activity when the protein is concentrated is calculated in terms of multiplying factor based on the value when the protein is non-concentrated.

### (6) Method of Measuring Stability Against Shaking Stress

### 1) Stability of Anti-EGFR Monoclonal Antibody Against Shaking Stress

Variation in protein content before and after shaking stress is measured as protein content retention rate. Before shaking stress, absorbance is measured, and concentration of an anti-EGFR monoclonal antibody is thereby measured. Shaking stress is applied, then an insoluble precipitate in the case where insoluble aggregates are formed by the shaking stress is removed by centrifugation, after which the absorbance is measured to measure the concentration of the anti-EGFR monoclonal antibody, and protein rate content retention rate is determined.

### 2) Stability of Anti-TNFα Monoclonal Antibody Against Shaking Stress

Variation in protein content before and after shaking stress is measured as protein content retention rate. Before shaking stress is applied, absorbance is measured, and concentration of an anti-TNFα monoclonal antibody is thereby measured. Shaking stress is applied, then an insoluble precipitate in the case where insoluble aggregates are formed by the shaking stress is removed by centrifugation, after which the absorbance is measured to measure the concentration of the anti-TNFα monoclonal antibody, and protein content retention rate is determined.

### 3) Stability of Anti-IgE Monoclonal Antibody Against Shaking Stress

Variation in protein content before and after shaking is measured as protein content retention rate. Before shaking stress is applied, absorbance is measured, and concentration of an anti-IgE monoclonal antibody is thereby measured. Shaking stress is applied, then an insoluble precipitate in the case where insoluble aggregates are formed by the shaking stress is removed by centrifugation, after which absorbance is measured to measure the concentration of the anti-IgE monoclonal antibody, and protein content retention rate is determined.

### (7) Method of Measuring Rate of Change in Primary Structure of Protein

### 1) Peptide Mapping Method

A test method in which a protein is digested chemically or enzymatically, the resulting peptide fragments are separated and detected by liquid chromatography (LC) or the like, and, by comparison of chromatographic patterns obtained, variation in constituent amino acids is determined. The state of change in the primary structure of the protein can be calculated in terms of rate of change in the primary structure.

### (8) Method of Measuring Stability Against Thermal Stress

1) Differential scanning calorimetry

A test method in which temperatures of a reference substance and a sample are measured while giving a predetermined heat to them, and an endothermic reaction or exothermic reaction due to a change in the state of the sample is measured. Input/output of heat attendant on a structural transition of the protein can be measured directly, and a thermal denaturing temperature of the protein can be measured.

### (9) Method of Measuring Aggregation Inhibitory Properties

1) Shaking stress is applied to a complex of an anti-EGFR monoclonal antibody and to a control liquid in which a complex is not formed. In regard of samples before the stress and after the stress, the amount of insoluble aggregates precipitated upon centrifugation under the conditions to be specifically described in Examples later is determined as an amount obtained by subtracting the amount of the anti-EGFR monoclonal antibody remaining in the supernatant after the shaking from the total amount of the anti-EGFR monoclonal antibody before the shaking.

2) A complex of L-asparaginase with a polyamino acid and a control liquid are heated, and, in regard of samples before the heating and after the heating, the numbers of aggregates are each determined by use of a micro-flow imaging method.

3) In regard of the same samples as in 2) above, absorbance is measured and turbidity is determined.

4) Shaking stress is applied to a complex of anti-TNFα monoclonal antibody with polyglutamic acid and a control liquid in which a complex is not formed. In regard of samples before the shaking stress and after the shaking stress, the numbers of aggregates are each determined by a micro-flow imaging method.

5) Size exclusion chromatograph

Also called size exclusion chromatography, molecular sieve chromatography, and SEC (Size Exclusion Chromatography). A chromatography in which sifting based on the molecular size of a sample is used as a principle. Since protein molecules can diffuse into the inside of a carrier whereas aggregates cannot reach the inside of the carrier and flow away in the exterior of the carrier, the amount of the aggregates can be measured. In the measurement in Example 98 to be described later, the amount of soluble aggregates is measured as a peak area.

### EXAMPLES

Various embodiments will be described more in detail below by depicting Examples, but the present invention is not limited to these Examples.

1. Examples 1 to 72 and Comparative Examples 1 to 72 which depict concentratability by polyamino acid, and negative control Comparative Examples 7-2 and 27-2 which depict that concentration is impossible by the use of amino acid other than polyamino acid L-Asparaginase

### Example 1

In a 10 mM MOPS (3-(N-morpholino)propanesulfonic acid) buffer (pH 7.0), L-asparaginase (pI: 4.7, MW: 141 kDa) was prepared so as to attain a concentration of 0.5 mg/mL, and poly-L-lysine (MW: 4 kDa to 15 kDa) was added thereto in amounts of 0.05 to 1 part by mass based on 1 part by mass of L-asparaginase. Each of these prepared liquids was centrifuged, and 90% of the total volume of the centrifuged liquid was removed as supernatant, to obtain L-asparaginase-poly-L-lysine complex-containing aqueous suspensions.

### Example 2

The same operations as in Example 1 except for using a 10 mM MOPS buffer (pH 6.4) were conducted, to obtain L-asparaginase-poly-L-lysine complex-containing aqueous suspensions.

### Example 3

The same operations as in Example 1 except for using a 10 mM Tris-HCl buffer (pH 8.2) were conducted, to obtain L-asparaginase-poly-L-lysine complex-containing aqueous suspensions.

### Example 4

The same operations as in Example 1 except for using a 10 mM Tris-HCl buffer (pH 8.7) were conducted, to obtain L-asparaginase-poly-L-lysine complex-containing aqueous suspensions.

### Example 5

In a 10 mM citrate buffer (pH 3.4), L-asparaginase was prepared so as to attain a concentration of 0.5 mg/mL, and poly-L-glutamic acid (MW: 3 kDa to 15 kDa) was added thereto in amounts of 0.1 to 1 part by mass based on 1 part by mass of L-asparaginase. Each of these prepared liquids was centrifuged, and 90% of the total volume of the centrifuged liquid was removed as supernatant, to obtain L-asparaginase-poly-L-glutamic acid complex-containing aqueous suspensions.

### Example 6

In a 10 mM citrate buffer (pH 2.9), L-asparaginase was prepared so as to attain a concentration of 0.5 mg/mL, and poly-L-glutamic acid (MW: 3 kDa to 15 kDa) was added thereto in amounts of 0.3 to 2 parts by mass based on 1 part by mass of L-asparaginase. Each of these prepared liquids was centrifuged, and 90% of the total volume of the centrifuged liquid was removed as supernatant, to obtain L-asparaginase-poly-L-glutamic acid complex-containing aqueous suspensions.

### Example 7

The same operations as in Example 1 except for using poly-L-arginine (MW: 5 kDa to 15 kDa) were conducted, to obtain L-asparaginase-poly-L-arginine complex-containing aqueous suspensions.

### Example 8

In a 10 mM MOPS buffer (pH 7.0), L-asparaginase was prepared so as to attain a concentration of 14.7 mg/mL, and poly-L-lysine (MW: 4 kDa to 15 kDa) was added thereto in amounts of 0.025 to 0.5 part by mass based on 1 part by mass of L-asparaginase. Each of these prepared liquids was centrifuged, and 90% of the total volume of the centrifuged liquid was removed as supernatant, to obtain L-asparaginase-poly-L-lysine complex-containing aqueous suspensions.

### Example 9

In a 10 mM MOPS buffer (pH 7.0), L-asparaginase was prepared so as to attain a concentration of 0.5 mg/mL, and poly-L-lysine (MW: 1 kDa to 5 kDa) was added thereto in amounts of 0.5 to 7 parts by mass based on 1 part by mass of L-asparaginase. Each of these prepared liquids was centrifuged, and 90% of the total volume of the centrifuged liquid was removed as supernatant, to obtain L-asparaginase-poly-L-lysine complex-containing aqueous suspensions.

### Example 10

In a 10 mM MOPS buffer (pH 7.0), L-asparaginase was prepared so as to attain a concentration of 1.0 mg/mL, and poly-L-lysine (MW: 15 kDa to 30 kDa) was added thereto in amounts of 0.005 to 0.3 part by mass based on 1 part by mass of L-asparaginase. Each of these prepared liquids was centrifuged, and 90% of the total volume of the centrifuged liquid was removed as supernatant, to obtain L-asparaginase-poly-L-lysine complex-containing aqueous suspensions.

### Example 11

The same operations as in Example 10 except for using poly-L-lysine (MW: not less than 30 kDa) were conducted, to obtain L-asparaginase-poly-L-lysine complex-containing aqueous suspension.

### Example 12

In a 10 mM HEPES (4-(2-hydroxyethyl)-1-piperazineethanesulfonic acid), L-asparaginase was prepared so as to attain a concentration of 1.5 mg/mL, and poly-L-lysine (MW: 4 kDa to 15 kDa) was added thereto in amounts of 0.05 to 0.5 part by mass based on 1 part by mass of L-asparaginase. Each of these prepared liquids was centrifuged, and 90% of the total volume of the centrifuged liquid was removed as supernatant, to obtain L-asparaginase-poly-L-lysine complex-containing aqueous suspensions.

### Example 13

The same operations as in Example 12 except for using a 10 mM MES (2-morpholinoethanesulfonic acid monohydrate) buffer (pH 7.0) were conducted, to obtain L-asparaginase-poly-L-lysine complex-containing aqueous suspensions.

### Human Serum Albumin

### Example 14

In a 10 mM MOPS buffer (pH 7.0), human serum albumin (pI: 5.0, MW: 69 kDa) was prepared so as to attain a concentration of 1.0 mg/mL, and poly-L-lysine (MW: 4 kDa to 15 kDa) was added thereto in amounts of 0.05 to 0.5 part by mass based on 1 part by mass of human serum albumin. Each of these prepared liquids was centrifuged, and 90% of the total volume of the centrifuged liquid was removed as supernatant, to obtain human serum albumin-poly-L-lysine complex-containing aqueous suspensions.

### Example 15

The same operations as in Example 14 except for using poly-L-lysine (MW: 15 kDa to 30 kDa) were conducted, to obtain human serum albumin-poly-L-lysine complex-containing aqueous suspensions.

### Example 16

The same operations as in Example 14 except for using poly-L-lysine (MW: not less than 30 kDa) were conducted, to obtain human serum albumin-poly-L-lysine complex-containing aqueous suspensions.

### Bovine Serum Albumin

### Example 17

In a 10 mM MOPS buffer (pH 7.0), bovine serum albumin (pI: 5.0, MW: 69 kDa) was prepared so as to attain a concentration of 0.5 mg/mL, and poly-L-lysine (MW: 4 kDa to 15 kDa) was added thereto in amounts of 0.05 to 0.5 part by mass based on 1 part by mass of bovine serum albumin. Each of these prepared liquids was centrifuged, and 90% of the total volume of the centrifuged liquid was removed as supernatant, to obtain bovine serum albumin-poly-L-lysine complex-containing aqueous suspensions.

### Example 18

The same operations as in Example 17 except for using a 10 mM MOPS buffer (pH 6.4) were conducted, to obtain bovine serum albumin-poly-L-lysine complex-containing aqueous suspensions.

### Example 19

In a 10 mM MOPS buffer (pH 7.0), bovine serum albumin was prepared so as to attain a concentration of 49.7 mg/mL, and poly-L-lysine (MW: 4 kDa to 15 kDa) was added thereto in amounts of 0.03 to 0.15 part by mass based on 1 part by mass of bovine serum albumin. Each of these prepared liquids was centrifuged, and 90% of the total volume of the centrifuged liquid was removed as supernatant, to obtain bovine serum albumin-poly-L-lysine complex-containing aqueous suspensions.

### <INF-α-2b>

### Example 20

In a 10 mM MOPS buffer (pH 7.5), interferon (INF)-α-2b (pI: 5.7, MW: 18 kDa) was prepared so as to attain a concentration of 0.04 mg/mL, and poly-L-lysine (MW: 4 kDa to 15 kDa) was added thereto in amounts of 0.05 to 0.5 part by mass based on 1 part by mass of INF-α-2b. Each of these prepared liquids was centrifuged, and 90% of the total volume of the centrifuged liquid was removed as supernatant, to obtain INF-α-2b-poly-L-lysine complex-containing aqueous suspensions.

### Example 21

In a 10 mM MOPS buffer (pH 7.0), INF-α-2b was prepared so as to attain a concentration of 0.1 mg/mL, and poly-L-lysine (MW: 4 kDa to 15 kDa) was added thereto in amounts of 0.05 to 0.5 part by mass based on 1 part by mass of INF-α-2b. Each of these prepared liquids was centrifuged, and 90% of the total volume of the centrifuged liquid was removed as supernatant, to obtain INF-α-2b-poly-L-lysine complex-containing aqueous suspensions.

### <Human Atrial Natriuretic Peptide>

### Example 22

In a 10 mM Tris-HCl buffer (pH 9.0), human atrial natriuretic peptide (pI: 10.5, MW: 3 kDa) was prepared so as to attain a concentration of 0.5 mg/mL, and poly-L-glutamic acid (MW: 0.75 kDa to 5 kDa) was added thereto in amounts of 0.05 to 1 part by mass based on 1 part by mass of human atrial natriuretic peptide. Each of these prepared liquids was centrifuged, and 90% of the total volume of the centrifuged liquid was removed as supernatant, to obtain human atrial natriuretic peptide-poly-L-glutamic acid complex-containing aqueous suspensions.

### Example 23

The same operations as in Example 22 except for using a 10 mM MOPS buffer (pH 7.0) were conducted, to obtain human atrial natriuretic peptide-poly-L-glutamic acid complex-containing aqueous suspensions.

### <Bovine Thyroglobulin>

### Example 24

In a 10 mM MOPS buffer (pH 7.0), bovine thyroglobulin (pI: 5.5, MW: 670 kDa) was prepared so as to attain a concentration of 0.12 mg/mL, and poly-L-lysine (MW: 4 kDa to 15 kDa) was added thereto in amounts of 0.05 to 1 part by mass based on 1 part by mass of bovine thyroglobulin. Each of these prepared liquids was centrifuged, and 90% of the total volume of the centrifuged liquid was removed as supernatant, to obtain bovine thyroglobulin-poly-L-lysine complex-containing aqueous suspensions.

### Example 25

The same operations as in Example 24 except for using poly-L-lysine (MW: not less than 30 kDa) were conducted, to obtain bovine thyroglobulin-poly-L-lysine complex-containing aqueous suspensions.

### <Anti-TNFα Monoclonal Antibody>

### Example 26

In a 10 mM MOPS buffer (pH 5.5), anti-tumor necrosis factor (TNF)-α monoclonal antibody (pI: 8.7, MW: 150 kDa) was prepared so as to attain a concentration of 0.5 mg/mL, and poly-L-glutamic acid (MW: 3 kDa to 15 kDa) was added thereto in amounts of 0.05 to 2 parts by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Example 27

In a 10 mM MOPS buffer (pH 5.5), anti-TNFα monoclonal antibody was prepared so as to attain a concentration of 0.5 mg/mL, and poly-DL-aspartic acid (MW: 2 kDa to 11 kDa) was added thereto in amounts of 0.05 to 1 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-DL-aspartic acid complex-containing aqueous suspensions.

### Example 28

In a 10 mM MOPS buffer (pH 5.5), anti-TNFα monoclonal antibody was prepared so as to attain a concentration of 0.5 mg/mL, and poly-L-glutamic acid (MW: 0.75 kDa to 5 kDa) was added thereto in amounts of 0.5 to 7 parts by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Example 29

In a 10 mM Tris-HCl buffer (pH 8.2), anti-TNFα monoclonal antibody was prepared so as to attain a concentration of 0.5 mg/mL, and poly-L-glutamic acid (MW: 3 kDa to 15 kDa) was added thereto in amounts of 0.05 to 1 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Example 30

The same operations as in Example 29 except for using a 10 mM MOPS buffer (pH 7.7) were conducted, to obtain anti-TNFα monoclonal antibody-poly-L-glutamic acid complex-containing aqueous suspensions.

### Example 31

The same operations as in Example 29 except for using a 10 mM MOPS buffer (pH 6.5) were conducted, to obtain anti-TNFα monoclonal antibody-poly-L-glutamic acid complex-containing aqueous suspensions.

### Example 32

In a 10 mM citrate buffer (pH 4.7), anti-TNFα monoclonal antibody was prepared so as to attain a concentration of 0.5 mg/mL, and poly-L-glutamic acid (MW: 3 kDa to 15 kDa) was added thereto in amounts of 0.1 to 1 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Example 33

In a 10 mM MOPS buffer (pH 7.0), anti-TNFα monoclonal antibody was prepared so as to attain a concentration of 0.5 mg/mL, and poly-L-glutamic acid (MW: 15 kDa to 50 kDa) was added thereto in amounts of 0.01 to 1 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Example 34

The same operations as in Example 33 except for using poly-L-glutamic acid (MW: 50 kDa to 100 kDa) were conducted, to obtain anti-TNFα monoclonal antibody-poly-L-glutamic acid complex-containing aqueous suspensions.

### Example 35

In a 10 mM phosphate buffer (pH 7.0), anti-TNFα monoclonal antibody was prepared so as to attain a concentration of 0.5 mg/mL, and poly-L-glutamic acid (MW: 3 kDa to 15 kDa) was added thereto in amounts of 0.05 to 0.5 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Example 36

The same operations as in Example 35 except for using a 10 mM PIPES (piperazine-1,4-bis(2-ethanesulfonic acid)) buffer (pH 7.0) were conducted, to obtain anti-TNFα monoclonal antibody-poly-L-glutamic acid complex-containing aqueous suspensions.

### Example 37

The same operations as in Example 35 except for using a 10 mM MES buffer (pH 7.0) were conducted, to obtain anti-TNFα monoclonal antibody-poly-L-glutamic acid complex-containing aqueous suspensions.

### Example 38

The same operations as in Example 35 except for using a 10 mM HEPES buffer (pH 7.0) were conducted, to obtain anti-TNFα monoclonal antibody-poly-L-glutamic acid complex-containing aqueous suspensions.

### <Anti-IgE Monoclonal Antibody>

### Example 39

In a 10 mM MOPS buffer (pH 5.5), anti-immunoglobulin E (IgE) monoclonal antibody (pI: 7.6, MW: 150 kDa) was prepared so as to attain a concentration of 0.5 mg/mL, and poly-L-glutamic acid (MW: 3 kDa to 15 kDa) was added thereto in amounts of 0.025 to 0.5 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspensions.

### Example 40

The same operations as in Example 39 except for using a 10 mM MOPS buffer (pH 6.5) were conducted, to obtain anti-IgE monoclonal antibody-poly-L-glutamic acid complex-containing aqueous suspensions.

### Example 41

In a 10 mM MOPS buffer (pH 5.5), anti-IgE monoclonal antibody was prepared so as to attain a concentration of 0.5 mg/mL, and poly-DL-aspartic acid (MW: 2 kDa to 11 kDa) was added thereto in amounts of 0.03 to 1 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-DL-aspartic acid complex-containing aqueous suspensions.

### Example 42

In a 10 mM MOPS buffer (pH 5.5), anti-IgE monoclonal antibody was prepared so as to attain a concentration of 15.0 mg/mL, and poly-L-glutamic acid (MW: 3 kDa to 15 kDa) was added thereto in amounts of 0.05 to 0.3 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspensions.

### Example 43

In a 10 mM citrate buffer (pH 5.4), anti-IgE monoclonal antibody was prepared so as to attain a concentration of 0.5 mg/mL, and poly-L-glutamic acid (MW: 3 kDa to 15 kDa) was added thereto in amounts of 0.05 to 1 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspensions.

### Example 44

In a 10 mM citrate buffer (pH 4.1), anti-IgE monoclonal antibody was prepared so as to attain a concentration of 0.5 mg/mL, and poly-L-glutamic acid (MW: 3 kDa to 15 kDa) was added thereto in amounts of 0.1 to 1 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspensions.

### Example 45

The same operations as in Example 44 except for using a 10 mM citrate buffer (pH 3.6) were conducted, to obtain anti-IgE monoclonal antibody-poly-L-glutamic acid complex-containing aqueous suspensions.

### Example 46

In a 10 mM Tris-HCl buffer (pH 8.2), anti-IgE monoclonal antibody was prepared so as to attain a concentration of 0.5 mg/mL, and poly-L-lysine (MW: 4 kDa to 15 kDa) was added thereto in amounts of 0.025 to 0.5 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-lysine complex-containing aqueous suspensions.

### Example 47

In a 10 mM Tris-HCl buffer (pH 9.0), anti-IgE monoclonal antibody was prepared so as to attain a concentration of 1.0 mg/mL, and poly-L-lysine (MW: 4 kDa to 15 kDa) was added thereto in amounts of 0.03 to 0.15 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-lysine complex-containing aqueous suspensions.

### Example 48

The same operations as in Example 47 except for using poly-L-lysine (MW: 15 kDa to 30 kDa) were conducted, to obtain anti-IgE monoclonal antibody-poly-L-lysine complex-containing aqueous suspensions.

### Example 49

The same operations as in Example 47 except for using poly-L-lysine (MW: not less than 30 kDa) were conducted, to obtain anti-IgE monoclonal antibody-poly-L-lysine complex-containing aqueous suspensions.

### Example 50

The same operations as in Example 47 except for using poly-L-arginine (MW: 5 kDa to 15 kDa) were conducted, to obtain anti-IgE monoclonal antibody-poly-L-arginine complex-containing aqueous suspensions.

### <Anti-EGFR Monoclonal Antibody>

### Example 51

In a 10 mM MOPS buffer (pH 5.0), anti-epidermal growth factor receptor (EGFR) monoclonal antibody (pI: 6.9, MW: 150 kDa) was prepared so as to attain a concentration of 1.0 mg/mL, and poly-L-glutamic acid (MW: 3 kDa to 15 kDa) was added thereto in amounts of 0.01 to 0.5 part by mass based on 1 part by mass of anti-EGFR monoclonal antibody. Each of these prepared liquids was centrifuged, and 90% of the total volume of the centrifuged liquid was removed as supernatant, to obtain anti-EGFR monoclonal antibody-poly-L-glutamic acid complex-containing aqueous suspensions.

### Example 52

The same operations as in Example 51 except for using a 10 mM MOPS buffer (pH 5.5) were conducted, to obtain anti-EGFR monoclonal antibody-poly-L-glutamic acid complex-containing aqueous suspensions.

### Example 53

In a 10 mM citrate buffer (pH 4.7), anti-EGFR monoclonal antibody was prepared so as to attain a concentration of 0.5 mg/mL, and poly-L-glutamic acid (MW: 3 kDa to 15 kDa) was added thereto in amounts of 0.05 to 1 part by mass based on 1 part by mass of anti-EGFR monoclonal antibody. Each of these prepared liquids was centrifuged, and 90% of the total volume of the centrifuged liquid was removed as supernatant, to obtain anti-EGFR monoclonal antibody-poly-L-glutamic acid complex-containing aqueous suspensions.

### Example 54

The same operations as in Example 53 except for using a 10 mM MES buffer (pH 4.7) were conducted, to obtain anti-EGFR monoclonal antibody-poly-L-glutamic acid complex-containing aqueous suspensions.

### Example 55

In a 10 mM citrate buffer (pH 3.4), anti-EGFR monoclonal antibody was prepared so as to attain a concentration of 0.5 mg/mL, and poly-L-glutamic acid (MW: 3 kDa to 15 kDa) was added thereto in amounts of 0.3 to 1 part by mass based on 1 part by mass of anti-EGFR monoclonal antibody. Each of these prepared liquids was centrifuged, and 90% of the total volume of the centrifuged liquid was removed as supernatant, to obtain anti-EGFR monoclonal antibody-poly-L-glutamic acid complex-containing aqueous suspensions.

### Example 56

In a 10 mM citrate buffer (pH 2.9), anti-EGFR monoclonal antibody was prepared so as to attain a concentration of 0.5 mg/mL, and poly-L-glutamic acid (MW: 3 kDa to 15 kDa) was added thereto in amounts of 0.5 to 1.5 parts by mass based on 1 part by mass of anti-EGFR monoclonal antibody. Each of these prepared liquids was centrifuged, and 90% of the total volume of the centrifuged liquid was removed as supernatant, to obtain anti-EGFR monoclonal antibody-poly-L-glutamic acid complex-containing aqueous suspensions.

### Example 57

In a 10 mM MOPS buffer (pH 7.5), anti-EGFR monoclonal antibody was prepared so as to attain a concentration of 0.5 mg/mL, and poly-L-lysine (MW: 4 kDa to 15 kDa) was added thereto in amounts of 0.05 to 1 part by mass based on 1 part by mass of anti-EGFR monoclonal antibody. Each of these prepared liquids was centrifuged, and 90% of the total volume of the centrifuged liquid was removed as supernatant, to obtain anti-EGFR monoclonal antibody-poly-L-lysine complex-containing aqueous suspensions.

### Example 58

The same operations as in Example 57 except for using a 10 mM Tris-HCl buffer (pH 8.2) were conducted, to obtain anti-EGFR monoclonal antibody-poly-L-glutamic acid complex-containing aqueous suspensions.

### Example 59

The same operations as in Example 57 except for using a 10 mM Tris-HCl buffer (pH 8.7) were conducted, to obtain anti-EGFR monoclonal antibody-poly-L-glutamic acid complex-containing aqueous suspensions.

### <Anti-HER2 Monoclonal Antibody>

### Example 60

In a 10 mM MOPS buffer (pH 6.5), anti-human epidermal growth factor receptor (HER) 2 monoclonal antibody (pI: 8.7, MW: 150 kDa) was prepared so as to attain a concentration of 0.5 mg/mL, and poly-L-glutamic acid (MW: 3 kDa to 15 kDa) was added thereto in amounts of 0.05 to 1 part by mass based on 1 part by mass of anti-HER2 monoclonal antibody. Each of these prepared liquids was centrifuged, and 90% of the total volume of the centrifuged liquid was removed as supernatant, to obtain anti-HER2 monoclonal antibody-poly-L-glutamic acid complex-containing aqueous suspensions.

### Example 61

The same operations as in Example 60 except for using a 10 mM MOPS buffer (pH 7.7) were conducted, to obtain anti-HER2 monoclonal antibody-poly-L-glutamic acid complex-containing aqueous suspensions.

### Example 62

The same operations as in Example 60 except for using a 10 mM Tris-HCl buffer (pH 8.2) were conducted, to obtain anti-HER2 monoclonal antibody-poly-L-glutamic acid complex-containing aqueous suspensions.

### Example 63

In a 10 mM citrate buffer (pH 4.7), anti-HER2 monoclonal antibody was prepared so as to attain a concentration of 0.5 mg/mL, and poly-L-glutamic acid (MW: 3 kDa to 15 kDa) was added thereto in amounts of 0.1 to 1 part by mass based on 1 part by mass of anti-HER2 monoclonal antibody. Each of these prepared liquids was centrifuged, and 90% of the total volume of the centrifuged liquid was removed as supernatant, to obtain anti-HER2 monoclonal antibody-poly-L-glutamic acid complex-containing aqueous suspensions.

### <Anti-CD20 Monoclonal Antibody>

### Example 64

In a 10 mM MOPS buffer (pH 6.5), anti-CD20 monoclonal antibody (pI: 8.7, MW: 150 kDa) was prepared so as to attain a concentration of 0.5 mg/mL, and poly-L-glutamic acid (MW: 3 kDa to 15 kDa) was added thereto in amounts of 0.05 to 1 part by mass based on 1 part by mass of anti-CD20 monoclonal antibody. Each of these prepared liquids was centrifuged, and 90% of the total volume of the centrifuged liquid was removed as supernatant, to obtain anti-CD20 monoclonal antibody-poly-L-glutamic acid complex-containing aqueous suspensions.

### Example 65

The same operations as in Example 64 except for using a 10 mM Tris-HCl buffer (pH 8.7) were conducted, to obtain anti-CD20 monoclonal antibody-poly-L-glutamic acid complex-containing aqueous suspensions.

### Example 66

The same operations as in Example 64 except for using a 10 mM MOPS buffer (pH 7.7) were conducted, to obtain anti-CD20 monoclonal antibody-poly-L-glutamic acid complex-containing aqueous suspensions.

### Example 67

In a 10 mM citrate buffer (pH 4.7), anti-CD20 monoclonal antibody was prepared so as to attain a concentration of 0.5 mg/mL, and poly-L-glutamic acid (MW: 3 kDa to 15 kDa) was added thereto in amounts of 0.1 to 1 part by mass based on 1 part by mass of anti-CD20 monoclonal antibody. Each of these prepared liquids was centrifuged, and 90% of the total volume of the centrifuged liquid was removed as supernatant, to obtain anti-CD20 monoclonal antibody-poly-L-glutamic acid complex-containing aqueous suspensions.

### <Human Soluble TNF Receptor-Fc Fusion Protein>

### Example 68

In a 10 mM Tris-HCl buffer (pH 8.7), human soluble TNF receptor-Fc fusion protein (pI: 8.0, MW: 150 kDa) was prepared so as to attain a concentration of 1.0 mg/mL, and poly-L-lysine (MW: 4 kDa to 15 kDa) was added thereto in amounts of 0.025 to 0.5 part by mass based on 1 part by mass of human the soluble receptor-Fc fusion protein. Each of these prepared liquids was centrifuged, and 90% of the total volume of the centrifuged liquid was removed as supernatant, to obtain human soluble TNF receptor-Fc fusion protein-poly-L-lysine complex-containing aqueous suspensions.

### Example 69

The same operations as in Example 68 except for using poly-L-lysine (MW: 15 kDa to 30 kDa) were conducted, to obtain human soluble TNF receptor-Fc fusion protein-poly-L-lysine complex-containing aqueous suspensions.

### Example 70

The same operations as in Example 68 except for using poly-L-lysine (MW: not less than 30 kDa) were conducted, to obtain human soluble TNF receptor-Fc fusion protein-poly-L-lysine complex-containing aqueous suspensions.

### <Human Immunoglobulin G>

### Example 71

In a 10 mM MOPS buffer (pH 5.5), human immunoglobulin G (hIgG) (pI: 6.9, MW: 150 kDa) was prepared so as to attain a concentration of 1.0 mg/mL, and poly-L-glutamic acid (MW: 3 kDa to 15 kDa) was added thereto in amounts of 0.01 to 0.5 part by mass based on 1 part by mass of the hIgG. Each of these prepared liquids was centrifuged, and 90% of the total volume of the centrifuged liquid was removed as supernatant, to obtain hIgG-poly-L-glutamic acid complex-containing aqueous suspensions.

### Example 72

The same operations as in Example 71 except for using poly-DL-aspartic acid (MW: 2 kDa to 11 kDa) were conducted, to obtain hIgG-poly-DL-aspartic acid complex-containing aqueous suspensions.

### Comparative Examples 1 to 72

The same operations as in Examples 1 to 72 except for not adding the polyamino acid in Examples 1 to 72 were conducted.

### Test Example 1

In regard of Examples 1 to 72 above, sodium chloride was added to each of the above-obtained protein-polyamino acid complex-containing aqueous suspension so as to attain a concentration of 150 mM, and absorbance at a wavelength of 280 nm was measured, whereby the protein concentration was measured. In addition, in regard of Comparative Examples 1 to 72 above, absorbance at a wavelength of 280 nm was measured, whereby the protein concentration was measured. From these results, the ratio of the protein concentration in each Example to the protein concentration in each Comparative Examples was determined. In all of Examples 1 to 72, a rise in protein concentration was found, which depicted that the protein can be concentrated. Details of the results are set forth in Tables 1 to 7.

TABLE 1

| | | Comparative Example | Example | | | | | | |
|---|---|---|---|---|---|---|---|---|---|
| Example 1 | Polyamino acid/protein mass ratio | 0 | 0.05 | 0.1 | 0.15 | 0.3 | 0.5 | 0.7 | 1 |
| | Protein concentration ratio | 1 | 10.5 | 9.8 | 9 | 5.8 | 3.2 | 2.1 | 1.3 |
| Example 2 | Polyamino acid/protein mass ratio | 0 | 0.05 | 0.1 | 0.15 | 0.3 | 0.5 | 0.7 | 1 |
| | Protein concentration ratio | 1 | 9.9 | 8.3 | 7 | 2.7 | 1.8 | 1.3 | 1 |
| Example 3 | Polyamino acid/protein mass ratio | 0 | 0.05 | 0.1 | 0.3 | 0.5 | 1 | | |
| | Protein concentration ratio | 1 | 9.6 | 9.8 | 9.2 | 8.1 | 3.9 | | |
| Example 4 | Polyamino acid/protein mass ratio | 0 | 0.05 | 0.1 | 0.3 | 0.5 | 1 | | |
| | Protein concentration ratio | 1 | 9.3 | 9.5 | 9.5 | 8.8 | 5.8 | | |
| Example 5 | Polyamino acid/protein mass ratio | 0 | 0.1 | 0.3 | 0.5 | 1 | | | |
| | Protein concentration ratio | 1 | 2.2 | 5.8 | 9.8 | 10.2 | | | |
| Example 6 | Polyamino acid/protein mass ratio | 0 | 0.3 | 0.5 | 1 | 2 | | | |
| | Protein concentration ratio | 1 | 1.1 | 1.7 | 5.1 | 5.4 | | | |
| Example 7 | Polyamino acid/protein mass ratio | 0 | 0.05 | 0.1 | 0.15 | 0.3 | 0.5 | 0.7 | 1 |
| | Protein concentration ratio | 1 | 9.7 | 8.2 | 7.2 | 2.1 | 1.1 | 1.2 | 1.2 |
| Example 8 | Polyamino acid/protein mass ratio | 0 | 0.025 | 0.05 | 0.1 | 0.15 | 0.3 | 0.5 | |
| | Protein concentration ratio | 1 | 11 | 11.1 | 9.5 | 8.3 | 3.8 | 1.7 | |
| Example 9 | Polyamino acid/protein mass ratio | 0 | 0.5 | 1 | 1.5 | 2 | 2.5 | 3 | 3.5 |
| | Protein concentration ratio | 1 | 6.3 | 7.9 | 8.2 | 7.8 | 7 | 4.8 | 3.8 |
| | Polyamino acid/protein mass ratio | | 4 | 4.5 | 5 | 5.5 | 6 | 7 | |
| | Protein concentration ratio | | 3.3 | 2.9 | 2.5 | 2.2 | 2 | 1.6 | |

TABLE 2

| | | Comparative | | | | | | | |
|---|---|---|---|---|---|---|---|---|---|
| | | Example | Example | | | | | | |
| Example 10 | Polyamino acid/protein mass ratio | 0 | 0.005 | 0.01 | 0.02 | 0.05 | 0.1 | 0.15 | 0.3 |
| | Protein concentration ratio | 1 | 2.7 | 5.1 | 9.2 | 9.8 | 7.2 | 3.8 | 0.9 |
| Example 11 | Polyamino acid/protein mass ratio | 0 | 0.005 | 0.01 | 0.02 | 0.05 | 0.1 | 0.15 | 0.3 |
| | Protein concentration ratio | 1 | 3.2 | 5.3 | 9.4 | 9.3 | 2.4 | 0.8 | 0.9 |
| Example 12 | Polyamino acid/protein mass ratio | 0 | 0.05 | 0.1 | 0.2 | 0.3 | 0.5 | | |
| | Protein concentration ratio | 1 | 10 | 9.3 | 7 | 4.5 | 2.5 | | |
| Example 13 | Polyamino acid/protein mass ratio | 0 | 0.05 | 0.1 | 0.2 | 0.3 | 0.5 | | |
| | Protein concentration ratio | 1 | 10.5 | 9.8 | 8.3 | 6.2 | 3.3 | | |
| Example 14 | Polyamino acid/protein mass ratio | 0 | 0.05 | 0.1 | 0.15 | 0.3 | 0.5 | | |
| | Protein concentration ratio | 1 | 1.7 | 2.5 | 1 | 0.8 | 0.8 | | |
| Example 15 | Polyamino acid/protein mass ratio | 0 | 0.05 | 0.1 | 0.15 | 0.3 | 0.5 | | |
| | Protein concentration ratio | 1 | 6.3 | 2.8 | 0.8 | 0.8 | 0.8 | | |
| Example 16 | Polyamino acid/protein mass ratio | 0 | 0.05 | 0.1 | 0.15 | 0.3 | 0.5 | | |
| | Protein concentration ratio | 1 | 8.7 | 4.7 | 0.8 | 0.7 | 0.7 | | |
| Example 17 | Polyamino acid/protein mass ratio | 0 | 0.05 | 0.1 | 0.15 | 0.3 | 0.5 | | |
| | Protein concentration ratio | 1 | 6.3 | 8.2 | 4.3 | 1.2 | 1 | | |
| Example 18 | Polyamino acid/protein mass ratio | 0 | 0.05 | 0.1 | 0.15 | 0.3 | 0.5 | | |
| | Protein concentration ratio | 1 | 6.8 | 5.3 | 2.9 | 1.3 | 1.2 | | |

TABLE 3

| | | Comparative | | | | | | | |
|---|---|---|---|---|---|---|---|---|---|
| | | Example | Example | | | | | | |
| Example | Polyamino acid/protein mass ratio | 0 | 0.03 | 0.05 | 0.075 | 0.1 | 0.15 | | |
| 19 | Protein concentration ratio | 1 | 5 | 9.2 | 8.7 | 7 | 1.2 | | |
| Example | Polyamino acid/protein mass ratio | 0 | 0.05 | 0.1 | 0.15 | 0.3 | 0.5 | | |
| 20 | Protein concentration ratio | 1 | 5.8 | 6 | 6.2 | 5.9 | 3.2 | | |
| Example | Polyamino acid/protein mass ratio | 0 | 0.05 | 0.1 | 0.15 | 0.3 | 0.5 | | |
| 21 | Protein concentration ratio | 1 | 6.8 | 6.8 | 6.9 | 7 | 6.4 | | |
| Example | Polyamino acid/protein mass ratio | 0 | 0.05 | 0.1 | 0.3 | 0.5 | | | |
| 22 | Protein concentration ratio | 1 | 1.5 | 2.9 | 6.8 | 0.8 | | | |
| Example | Polyamino acid/protein mass ratio | 0 | 0.05 | 0.1 | 0.3 | 0.5 | | | |
| 23 | Protein concentration ratio | 1 | 3.4 | 5.6 | 9.9 | 7.9 | | | |
| Example | Polyamino acid/protein mass ratio | 0 | 0.05 | 0.1 | 0.15 | 0.3 | 0.5 | 0.7 | 1 |
| 24 | Protein concentration ratio | 1 | 7.7 | 7.9 | 8.1 | 7.6 | 7.6 | 7.7 | 6.7 |
| Example | Polyamino acid/protein mass ratio | 0 | 0.05 | 0.1 | 0.15 | 0.3 | 0.5 | 0.7 | 1 |
| 25 | Protein concentration ratio | 1 | 6.7 | 4.1 | 2.5 | 1.1 | 1.1 | 1 | 1.1 |
| Example | Polyamino acid/protein mass ratio | 0 | 0.05 | 0.1 | 0.2 | 0.3 | 0.5 | 1 | 2 |
| 26 | Protein concentration ratio | 1 | 4.6 | 10.3 | 10.4 | 8.3 | 7.5 | 7.4 | 4.9 |
| Example | Polyamino acid/protein mass ratio | 0 | 0.05 | 0.1 | 0.15 | 0.3 | 0.5 | 0.7 | 1 |
| 27 | Protein concentration ratio | 1 | 10.7 | 9.6 | 7.6 | 4.4 | 3.7 | 2.8 | 1.1 |
| Example | Polyamino acid/protein mass ratio | 0 | 0.5 | 1 | 1.5 | 2 | 2.5 | 3 | 3.5 |
| 28 | Protein concentration ratio | 1 | 9.4 | 10.3 | 10.1 | 10 | 9.3 | 8.8 | 8 |
| | Polyamino acid/protein mass ratio | | 4 | 4.5 | 5 | 5.5 | 6 | 6.5 | 7 |
| | Protein concentration ratio | | 7.1 | 5.9 | 4.4 | 2.9 | 1.9 | 1.3 | 1.1 |
| Example | Polyamino acid/protein mass ratio | 0 | 0.05 | 0.1 | 0.3 | 0.5 | 1 | | |
| 29 | Protein concentration ratio | 1 | 8.7 | 8.3 | 5.1 | 3.4 | 0.9 | | |

TABLE 4

| | | Comparative | | | | | | | |
|---|---|---|---|---|---|---|---|---|---|
| | | Example | Example | | | | | | |
| Example | Polyamino acid/protein mass ratio | 0 | 0.05 | 0.1 | 0.3 | 0.5 | 1 | | |
| 30 | Protein concentration ratio | 1 | 9.5 | 9.4 | 8.4 | 7.6 | 5 | | |
| Example | Polyamino acid/protein mass ratio | 0 | 0.05 | 0.1 | 0.3 | 0.5 | 1 | | |
| 31 | Protein concentration ratio | 1 | 10 | 9.6 | 8.9 | 8.9 | 6.4 | | |
| Example | Polyamino acid/protein mass ratio | 0 | 0.1 | 0.3 | 0.5 | 1 | | | |
| 32 | Protein concentration ratio | 1 | 7.1 | 9.7 | 10.1 | 9.9 | | | |
| Example | Polyamino acid/protein mass ratio | 0 | 0.01 | 0.025 | 0.05 | 0.075 | 0.1 | 0.15 | 0.3 |
| 33 | Protein concentration ratio | 1 | 5.9 | 10 | 9.9 | 9.9 | 9.7 | 8.7 | 6.9 |
| | Polyamino acid/protein mass ratio | | 0.5 | 1 | | | | | |
| | Protein concentration ratio | | 5.4 | 4.8 | | | | | |
| Example | Polyamino acid/protein mass ratio | 0 | 0.01 | 0.025 | 0.05 | 0.075 | 0.1 | 0.15 | 0.3 |
| 34 | Protein concentration ratio | 1 | 5.8 | 9.9 | 9.1 | 8 | 6.8 | 5.1 | 1.9 |
| | Polyamino acid/protein mass ratio | | 0.5 | 1 | | | | | |
| | Protein concentration ratio | | 1 | 0.9 | | | | | |
| Example | Polyamino acid/protein mass ratio | 0 | 0.05 | 0.1 | 0.2 | 0.3 | 0.5 | | |
| 35 | Protein concentration ratio | 1 | 8.3 | 8.6 | 8.5 | 8.3 | 7.5 | | |
| Example | Polyamino acid/protein mass ratio | 0 | 0.05 | 0.1 | 0.2 | 0.3 | 0.5 | | |
| 36 | Protein concentration ratio | 1 | 8.2 | 9 | 8.8 | 8.7 | 8.1 | | |
| Example | Polyamino acid/protein mass ratio | 0 | 0.05 | 0.1 | 0.2 | 0.3 | 0.5 | | |
| 37 | Protein concentration ratio | 1 | 7.4 | 9.2 | 8.9 | 8.5 | 8.2 | | |
| Example | Polyamino acid/protein mass ratio | 0 | 0.05 | 0.1 | 0.2 | 0.3 | 0.5 | | |
| 38 | Protein concentration ratio | 1 | 9.3 | 9.1 | 8.6 | 7.8 | 6.2 | | |
| Example | Polyamino acid/protein mass ratio | 0 | 0.025 | 0.05 | 0.1 | 0.15 | 0.3 | 0.5 | |
| 39 | Protein concentration ratio | 1 | 3.3 | 7 | 10 | 10.2 | 6.1 | 0.9 | |
| Example | Polyamino acid/protein mass ratio | 0 | 0.025 | 0.05 | 0.1 | 0.15 | 0.3 | 0.5 | |
| 40 | Protein concentration ratio | 1 | 5.7 | 7.3 | 2.5 | 0.6 | 0.7 | 0.8 | |

TABLE 5

| | | Comparative | | | | | | | |
|---|---|---|---|---|---|---|---|---|---|
| | | Example | Example | | | | | | |
| Example | Polyamino acid/protein mass ratio | 0 | 0.03 | 0.05 | 0.1 | 0.15 | 0.3 | 0.5 | 1 |
| 41 | Protein concentration ratio | 1 | 4.9 | 10.2 | 10.2 | 7.5 | 1 | 1.1 | 1.1 |
| Example | Polyamino acid/protein mass ratio | 0 | 0.05 | 0.1 | 0.15 | 0.3 | | | |
| 42 | Protein concentration ratio | 1 | 10.3 | 9.7 | 7.5 | 1.8 | | | |
| Example | Polyamino acid/protein mass ratio | 0 | 0.05 | 0.1 | 0.3 | 0.5 | 1 | | |
| 43 | Protein concentration ratio | 1 | 10.1 | 10.4 | 9.8 | 9.6 | 9.4 | | |
| Example | Polyamino acid/protein mass ratio | 0 | 0.1 | 0.3 | 0.5 | 1 | | | |
| 44 | Protein concentration ratio | 1 | 3.9 | 10.5 | 10.7 | 10.6 | | | |
| Example | Polyamino acid/protein mass ratio | 0 | 0.1 | 0.3 | 0.5 | 1 | | | |
| 45 | Protein concentration ratio | 1 | 0.9 | 6.3 | 9.4 | 9.4 | | | |
| Example | Polyamino acid/protein mass ratio | 0 | 0.025 | 0.05 | 0.1 | 0.15 | 0.3 | 0.5 | |
| 46 | Protein concentration ratio | 1 | 2.8 | 5 | 7 | 7.4 | 4.3 | 1 | |
| Example | Polyamino acid/protein mass ratio | 0 | 0.03 | 0.05 | 0.07 | 0.1 | 0.15 | | |
| 47 | Protein concentration ratio | 1 | 8.6 | 9.4 | 9.6 | 9.4 | 8.9 | | |
| Example | Polyamino acid/protein mass ratio | 0 | 0.03 | 0.05 | 0.07 | 0.1 | 0.15 | | |
| 48 | Protein concentration ratio | 1 | 9.7 | 10 | 9.8 | 9.5 | 8.8 | | |
| Example | Polyamino acid/protein mass ratio | 0 | 0.03 | 0.05 | 0.07 | 0.1 | 0.15 | | |
| 49 | Protein concentration ratio | 1 | 9.9 | 9.8 | 9.9 | 9.6 | 7.9 | | |
| Example | Polyamino acid/protein mass ratio | 0 | 0.03 | 0.05 | 0.07 | 0.1 | 0.15 | | |
| 50 | Protein concentration ratio | 1 | 10 | 10 | 10 | 9.5 | 8 | | |
| Example | Polyamino acid/protein mass ratio | 0 | 0.01 | 0.025 | 0.05 | 0.1 | 0.15 | 0.3 | 0.5 |
| 51 | Protein concentration ratio | 1 | 0.9 | 2.2 | 8.6 | 4.8 | 0.9 | 0.8 | 0.9 |
| Example | Polyamino acid/protein mass ratio | 0 | 0.1 | 0.025 | 0.05 | 0.1 | 0.15 | 0.3 | 0.5 |
| 52 | Protein concentration ratio | 1 | 1 | 3.9 | 8 | 2.5 | 0.9 | 0.8 | 0.9 |
| Example | Polyamino acid/protein mass ratio | 0 | 0.05 | 0.1 | 0.3 | 0.5 | 1 | | |
| 53 | Protein concentration ratio | 1 | 1.4 | 3.1 | 7.6 | 7.5 | 7.1 | | |

TABLE 6

| | | Comparative | | | | | | |
|---|---|---|---|---|---|---|---|---|
| | | Example | Example | | | | | |
| Example 54 | Polyamino acid/protein mass ratio | 0 | 0.05 | 0.1 | 0.15 | 0.3 | 0.5 | 1 |
| | Protein concentration ratio | 1 | 3 | 9.2 | 10 | 9.5 | 6.5 | 0.9 |
| Example 55 | Polyamino acid/protein mass ratio | 0 | 0.3 | 0.5 | 1 | | | |
| | Protein concentration ratio | 1 | 5.1 | 9.8 | 9.8 | | | |
| Example 56 | Polyamino acid/protein mass ratio | 0 | 0.5 | 1 | 1.25 | 1.5 | | |
| | Protein concentration ratio | 1 | 1.7 | 9.3 | 9.3 | 9.5 | | |
| Example 57 | Polyamino acid/protein mass ratio | 0 | 0.05 | 0.1 | 0.15 | 0.3 | 0.5 | 1 |
| | Protein concentration ratio | 1 | 5.6 | 8.3 | 7.7 | 6.2 | 2.9 | 0.9 |
| Example 58 | Polyamino acid/protein mass ratio | 0 | 0.05 | 0.1 | 0.15 | 0.3 | 0.5 | 1 |
| | Protein concentration ratio | 1 | 8.5 | 9.2 | 9.2 | 8.5 | 6.9 | 2.4 |
| Example 59 | Polyamino acid/protein mass ratio | 0 | 0.05 | 0.1 | 0.15 | 0.3 | 0.5 | 1 |
| | Protein concentration ratio | 1 | 9.7 | 10 | 9.9 | 9.2 | 8.2 | 4.3 |
| Example 60 | Polyamino acid/protein mass ratio | 0 | 0.05 | 0.15 | 0.5 | 1 | | |
| | Protein concentration ratio | 1 | 9.6 | 8.6 | 1.1 | 0.7 | | |
| Example 61 | Polyamino acid/protein mass ratio | 0 | 0.05 | 0.15 | 0.5 | 1 | | |
| | Protein concentration ratio | 1 | 8.2 | 4.8 | 0.7 | 0.7 | | |
| Example 62 | Polyamino acid/protein mass ratio | 0 | 0.05 | 0.15 | 0.5 | 1 | | |
| | Protein concentration ratio | 1 | 5.4 | 0.7 | 0.6 | 0.8 | | |
| Example 63 | Polyamino acid/protein mass ratio | 0 | 0.1 | 0.3 | 0.5 | 1 | | |
| | Protein concentration ratio | 1 | 6.4 | 9.2 | 9.3 | 9.4 | | |
| Example 64 | Polyamino acid/protein mass ratio | 0 | 0.05 | 0.15 | 0.5 | 1 | | |
| | Protein concentration ratio | 1 | 9.2 | 8.2 | 2.2 | 0.7 | | |
| Example 65 | Polyamino acid/protein mass ratio | 0 | 0.05 | 0.15 | 0.5 | 1 | | |
| | Protein concentration ratio | 1 | 7.4 | 4.7 | 0.7 | 0.7 | | |
| Example 66 | Polyamino acid/protein mass ratio | 0 | 0.05 | 0.15 | 0.5 | 1 | | |
| | Protein concentration ratio | 1 | 6.3 | 2 | 0.7 | 0.7 | | |

TABLE 7

| | | Comparative | | | | | | | |
|---|---|---|---|---|---|---|---|---|---|
| | | Example | Example | | | | | | |
| Example 67 | Polyamino acid/protein mass ratio | 0 | 0.1 | 0.3 | 0.5 | 1 | | | |
| | Protein concentration ratio | 1 | 6.9 | 9.8 | 10.2 | 9.8 | | | |
| Example 68 | Polyamino acid/protein mass ratio | 0 | 0.025 | 0.05 | 0.1 | 0.15 | 0.3 | 0.5 | |
| | Protein concentration ratio | 1 | 0.9 | 1 | 3.2 | 3 | 0.9 | 0.9 | |
| Example 69 | Polyamino acid/protein mass ratio | 0 | 0.025 | 0.05 | 0.1 | 0.15 | 0.3 | 0.5 | |
| | Protein concentration ratio | 1 | 0.9 | 0.9 | 6.3 | 3.2 | 0.8 | 0.8 | |
| Example 70 | Polyamino acid/protein mass ratio | 0 | 0.025 | 0.05 | 0.1 | 0.15 | 0.3 | 0.5 | |
| | Protein concentration ratio | 1 | 0.9 | 0.9 | 8.6 | 4.7 | 0.9 | 0.9 | |
| Example 71 | Polyamino acid/protein mass ratio | 0 | 0.01 | 0.025 | 0.05 | 0.1 | 0.15 | 0.3 | 0.5 |
| | Protein concentration ratio | 1 | 1.7 | 5.1 | 9.8 | 9.2 | 6.5 | 1.7 | 1 |
| Example 72 | Polyamino acid/protein mass ratio | 0 | 0.01 | 0.025 | 0.05 | 0.1 | 0.15 | 0.3 | 0.5 |
| | Protein concentration ratio | 1 | 1.7 | 5.9 | 10.3 | 9.7 | 7.3 | 2.2 | 1 |

### Comparative Example 7-2, Comparative Example 27-2

The same operations as in Example 7, except for using L-arginine in place of poly-L-arginine (MW: 5 kDa to 15 kDa) in Example 7, were conducted as a negative control (Comparative Example 7-2). Besides, the same operations as in Example 27, except for using aspartic acid in place of poly-DL-aspartic acid (MW: 2 kDa to 11 kDa) in Example 27, were conducted as a negative control (Comparative Example 27-2).

### Comparative Examples 7 and 27

As the negative control comparative examples, the same samples as in Comparative Example 7 and Comparative Example 27, except for adding neither an amino acid nor a polyamino acid, were produced.

### Test Example 2

In regard of Comparative Examples 7-2 and 27-2, sodium chloride was added to each of the obtained protein-amino acid-containing aqueous suspensions, so as to attain a concentration of 150 mM, and absorbance at a wavelength of 280 nm was measured, whereby the protein concentration was measured. Besides, in regard of Comparative Examples 7 and 27, absorbance at a wavelength of 280 nm was measured, whereby the protein concentration was measured. From these results, the ratio of protein concentration in each Example to protein concentration in each Comparative Example was determined. In negative-control Comparative Examples 7-2 and 27-2, a rise in protein concentration was not observed; thus, it was depicted that by the sole use of an amino acid which is not a polyamino acid, it is impossible to concentrate the protein. Details of the results were set forth in Table 8, together with the results of Examples 7 and 27 in which concentration of the protein was possible by a polyamino acid.

TABLE 8

| | | Comparative | | | | | | |
|---|---|---|---|---|---|---|---|---|
| | | Example | | | | | | |
| Example 7 | Polyamino acid/protein mass | 0 | 0.05 | 0.1 | 0.15 | 0.3 | 0.5 | 1 |
| | ratio | | | | | | | |
| | Protein concentration ratio | 1 | 9.7 | 8.2 | 7.2 | 2.1 | 1.1 | 1.2 |
| Comparative Example | Amino acid/protein mass ratio | 0 | 0.05 | 0.1 | 0.15 | 0.3 | 0.5 | 1 |
| 7-2 (negative control) | Protein concentration ratio | 1 | 1 | 1 | 1 | 1 | 1 | 1 |
| Example 27 | Polyamino acid/protein mass | 0 | 0.05 | 0.1 | 0.15 | 0.3 | 0.5 | 1 |
| | ratio | | | | | | | |
| | Protein concentration ratio | 1 | 10.7 | 9.6 | 7.6 | 4.4 | 3.7 | 1.1 |
| Comparative Example | Amino acid/protein mass ratio | 0 | 0.05 | 0.1 | 0.15 | 0.3 | 0.5 | 1 |
| 27-2 (negative control) | Protein concentration ratio | 1 | 1 | 1 | 1 | 1 | 1 | 1 |

### 2. Examples 73 to 76 (Other Proteins) which Depict Possibility of Concentration by Polyamino Acid

### <Darbepoetin α>

### Example 73

In a 10 mM glycine-NaOH buffer (pH 10.5), darbepoetin α (pI: 8.8, MW: 36 kDa) was prepared so as to attain a concentration of 0.12 mg/mL, and poly-L-lysine (MW: not less than 30 kDa) was added thereto in amounts of 0.1 to 2 parts by mass based on 1 part by mass of darbepoetin α. Each of these prepared liquids was centrifuged, and 90% of the total volume of the centrifuged liquid was removed as supernatant, to obtain darbepoetin α-poly-L-lysine complex-containing aqueous suspensions.

### <High-Concentration Anti-TNFα Monoclonal Antibody>

### Example 74

In a 10 mM MOPS buffer (pH 6.5), anti-TNFα monoclonal antibody was prepared so as to attain a concentration of 15.0 mg/mL, and poly-L-glutamic acid (MW: 3 kDa to 15 kDa) was added thereto in amounts of 0.02 to 0.5 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### <High-Concentration IgE Monoclonal Antibody>

### Example 75

In a 10 mM citrate buffer (pH 5.5), anti-IgE monoclonal antibody was prepared so as to attain a concentration of 15 mg/mL, and poly-L-glutamic acid (MW: 3 kDa to 15 kDa) was added thereto in amounts of 0.02 to 0.5 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspensions.

### <Anti-TNFα Monoclonal Antibody Fab Fragment>

### Example 76

In a 10 mM MOPS buffer (pH 6.5), tumor necrosis factor (TNF) a monoclonal antibody Fab fragment (pI: 8.8, MW: 110 kDa) was prepared so as to attain a concentration of 1.16 mg/mL, and poly-L-glutamic acid (MW: 3 kDa to 15 kDa) was added thereto in amounts of 0.025 to 0.5 part by mass based on 1 part by mass of anti-TNFα monoclonal antibody Fab fragment. Each of these prepared liquids was centrifuged, and 90% of the total volume of the centrifuged liquid was removed as supernatant, to obtain anti-TNFα monoclonal antibody Fab fragment-poly-L-glutamic acid complex-containing aqueous suspensions.

TABLE 9

| | | Comparative | | | | | | |
|---|---|---|---|---|---|---|---|---|
| | | Example | | | | | | |
| Example 73 | Polyamino acid/protein mass | 0 | 0.1 | 0.3 | 0.5 | 1 | 1.5 | 2 |
| | ratio | | | | | | | |
| | Protein concentration ratio | 1 | 0.6 | 1.4 | 2.7 | 5.9 | 6.2 | 6.0 |
| Example 74 | Polyamino acid/protein mass | 0 | 0.02 | 0.05 | 0.1 | 0.3 | 0.5 | |
| | ratio | | | | | | | |
| | Protein concentration ratio | 1 | 7.7 | 10.0 | 10.0 | 8.3 | 1.1 | |
| Example 75 | Polyamino acid/protein mass | 0 | 0.02 | 0.05 | 0.1 | 0.3 | 0.5 | |
| | ratio | | | | | | | |
| | Protein concentration ratio | 1 | 9.9 | 10.1 | 10.2 | 9.9 | 8.8 | |
| Example 76 | Polyamino acid/protein mass | 0 | 0.025 | 0.05 | 0.1 | 0.15 | 0.3 | 0.5 |
| | ratio | | | | | | | |
| | Protein concentration ratio | 1 | 8.8 | 9.8 | 8.6 | 7.7 | 4.5 | 2.7 |

### 3. Concentration of Protein has Maximum Concentration Factor

In the method of concentrating a protein through forming the disclosed complex, it is seen that when concentration factor is tabulated in relation to pI and pH of the buffer used, the table depicts maximum concentration factors.

Referring to Table 10 below, the pH of a buffer was varied in relation to isoelectric point pI of each of proteins being concentrated in Tables 1 to 7, and, in a condition where the absolute value (|pI−pH|) of pI−pH is at the value as depicted in Table 10, the protein and a polyamino acid were added to the buffer to form a complex of the protein and the polyamino acid. The concentration of the protein in the complex obtained was measured, and the value of |pI−pH| at which a maximum value of the concentration factor is obtained was depicted.

Plotting the concentration factor against |pI−pH| gives the graphs depicted in FIG. 5. FIG. 5 depicts that a maximum concentration factor for the protein in the complex is present where |pI−pH| is in the range from 1.5 to 4.0.

The values of |pI−pH| at which the maximum values of concentration factor can be obtained are as follows.

Anti-TNFα monoclonal antibody-poly-L-glutamic acid: 2.2
Anti-IgE monoclonal antibody-poly-L-glutamic acid: 2.2
Anti-EGFR monoclonal antibody-poly-L-glutamic acid: 2.2
L-asparaginase-poly-L-lysine: 2.3

TABLE 10

| | | | Buffer | | |
|---|---|---|---|---|---|
| Protein | Concentration factor | pI-pH | Kind | pH | Polyglutamic acid |
| Asparaginase | | 0.5 | Citrate | 5.2 | Polylysine |
| Asparaginase | | 1.0 | Citrate | 5.7 | Polylysine |
| Asparaginase | Maximum | 2.3 | MOPS | 7.0 | Polylysine |
| Asparaginase | | 3.5 | Tris-HCl | 8.2 | Polylysine |
| Asparaginase | | 4.0 | Tris-HCl | 8.7 | Polylysine |
| Anti-TNFα antibody | | 0.5 | Tris-HCl | 8.2 | Polyglutamic acid |
| Anti-TNFα antibody | | 1.0 | Tris-HCl | 7.7 | Polyglutamic acid |
| Anti-TNFα antibody | Maximum | 2.2 | MOPS | 6.5 | Polyglutamic acid |
| Anti-TNFα antibody | | 3.5 | MOPS | 5.2 | Polyglutamic acid |
| Anti-TNFα antibody | | 4 | Citrate | 4.7 | Polyglutamic acid |
| Anti-IgE antibody | | 4.0 | Citrate | 3.6 | Polyglutamic acid |
| Anti-IgE antibody | | 3.5 | Citrate | 4.1 | Polyglutamic acid |
| Anti-IgE antibody | Maximum | 2.2 | Citrate | 5.4 | Polyglutamic acid |
| Anti-IgE antibody | | 1.0 | MOPS | 6.6 | Polyglutamic acid |
| Anti-IgE antibody | | 0.5 | MOPS | 7.1 | Polyglutamic acid |
| Anti-EGFR antibody | | 4.0 | Citrate | 2.9 | Polyglutamic acid |
| Anti-EGFR antibody | | 3.5 | Citrate | 3.4 | Polyglutamic acid |
| Anti-EGFR antibody | Maximum | 2.2 | Citrate | 4.7 | Polyglutamic acid |
| Anti-EGFR antibody | | 1.9 | Citrate | 5.0 | Polyglutamic acid |
| Anti-EGFR antibody | | 1.0 | Citrate | 5.9 | Polyglutamic acid |
| Anti-EGFR antibody | | 0.5 | MOPS | 6.4 | Polyglutamic acid |

### 4. Examples 77 to 86 which Depict that Activity is not Damaged by Concentration of Protein

### <Anti-IgE Monoclonal Antibody>

### Example 77

In a 10 mM MOPS buffer (pH 5.5), anti-IgE monoclonal antibody was prepared so as to attain a concentration of 0.5 mg/mL, and poly-L-glutamic acid (MW: 3 kDa to 15 kDa) was added thereto in amounts of 0.25, 0.05, 0.1, and 0.15 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspensions.

### Comparative Example 77

The same operations as in Example 77, except for not adding poly-L-glutamic acid, were conducted.

### Test Example 3

In regard of Example 77 above, sodium chloride was added to the obtained anti-IgE monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-IgE monoclonal antibody was measured. Besides, in regard of Comparative Example 77, absorbance at a wavelength of 280 nm was measured as measurement of protein concentration, and, further, the activity of anti-IgE monoclonal antibody was measured. From these results, the ratio of the protein concentration in Example 77 to the protein concentration in Comparative Example 77, and the ratio of the anti-IgE monoclonal antibody activity in Example 77 to the anti-IgE monoclonal antibody activity in Comparative Example 77 were determined. It was found that both the protein concentration ratio and the activity ratio rose to about 10 times, depicting that the anti-IgE monoclonal antibody can be concentrated without damaging the activity thereof. The results were set forth in Table 11.

TABLE 11

| | | Comparative | | | | |
|---|---|---|---|---|---|---|
| | | Example 77 | Example 77 | | | |
| Test | Polyamino | 0 | 0.025 | 0.05 | 0.1 | 0.15 |
| Example 3 | acid/protein mass | | | | | |
| | ratio | | | | | |
| | Protein | 1 | 5.4 | 9.2 | 10.2 | 9.1 |
| | concentration | | | | | |
| | ratio | | | | | |
| | Activity ratio | 1 | 5.5 | 9 | 9.6 | 9.1 |

### Example 78

In a 10 mM MOPS buffer (pH 5.5), anti-IgE monoclonal antibody was prepared so as to attain a concentration of 15 mg/mL, and poly-L-glutamic acid (MW: 3 kDa to 15 kDa) was added thereto in amounts of 0.05, 0.10, and 0.15 part by mass based on 1 part by mass of anti-IgE monoclonal antibody. Each of these prepared liquids was centrifuged, and 90% of the total volume of the centrifuged liquid was removed as supernatant, to obtain anti-IgE monoclonal antibody-poly-L-glutamic acid complex-containing aqueous suspension.

### Comparative Example 78

The same operations as in Example 78, except for not adding poly-L-glutamic acid, were conducted.

### Test Example 4

In regard of Example 78 above, sodium chloride was added to the obtained anti-IgE monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-IgE monoclonal antibody was measured. Besides, in regard of Comparative Example 78, absorbance at a wavelength of 280 nm was measured as measurement of protein concentration, and, further, the activity of anti-IgE monoclonal antibody was measured. From the results of these measurements, the ratio of the protein concentration in Example 78 to the protein concentration in Comparative Example 78, and the ratio of the anti-IgE monoclonal antibody activity in Example 78 to the anti-IgE monoclonal antibody activity in Comparative Example 78 were determined. It was found that both the protein concentration ratio and the activity ratio rose to about 10 times, depicting that the anti-IgE monoclonal antibody can be concentrated without damaging the activity thereof. The results were set forth in Table 12.

TABLE 12

| | | Comparative | | | |
|---|---|---|---|---|---|
| | | Example 78 | Example 78 | | |
| Test | Polyamino acid/protein | 0 | 0.05 | 0.1 | 0.15 |
| Example 4 | mass ratio | | | | |
| | Protein concentration | 1 | 10.3 | 9.7 | 7.5 |
| | ratio | | | | |
| | Activity ratio | 1 | 10.5 | 10.2 | 7.3 |

### Example 79

In a 10 mM citrate buffer (pH 5.5), anti-IgE monoclonal antibody was prepared so as to attain a concentration of 15 mg/mL, and poly-L-glutamic acid (MW: 3 kDa to 15 kDa) was added thereto in an amount of 0.05 part by mass based on 1 part by mass of anti-IgE monoclonal antibody. Each of these prepared liquids was centrifuged, and 90% of the total volume of the centrifuged liquid was removed as supernatant, to obtain an anti-IgE monoclonal antibody-poly-L-glutamic acid complex-containing aqueous suspension.

### Comparative Example 79

The same operations as in Example 79, except for not adding poly-L-glutamic acid, were conducted.

### Test Example 5

In regard of Example 79 above, sodium chloride was added to the obtained anti-IgE monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-IgE monoclonal antibody was measured. Besides, in regard of Comparative Example 79, absorbance at a wavelength of 280 nm was measured as measurement of protein concentration, and, further, the activity of anti-IgE monoclonal antibody was measured. From the results of these measurements, the ratio of the protein concentration in Example 79 to the protein concentration in Comparative Example 79, and the ratio of the anti-IgE monoclonal antibody activity in Example 79 to the anti-IgE monoclonal antibody activity in Comparative Example 79 were determined. It was found that both the protein concentration ratio and the activity ratio rose to about 10 times, depicting that the anti-IgE monoclonal antibody can be concentrated without damaging the activity thereof. The results were set forth in Table 13.

TABLE 13

| | | Comparative | Example |
|---|---|---|---|
| | | Example 79 | 79 |
| Test Example 5 | Polyamino acid/protein mass | 0 | 0.5 |
| | ratio | | |
| | Protein concentration ratio | 1 | 10.1 |
| | Activity ratio | 1 | 10.6 |

### Example 80

In a 10 mM citrate buffer (pH 3.6), anti-IgE monoclonal antibody was prepared so as to attain a concentration of 0.5 mg/mL, and poly-L-glutamic acid (MW: 3 kDa to 15 kDa) was added thereto in an amount of 0.6 part by mass based on 1 part by mass of anti-IgE monoclonal antibody. Each of these prepared liquids was centrifuged, and 90% of the total volume of the centrifuged liquid was removed as supernatant, to obtain each anti-IgE monoclonal antibody-poly-L-glutamic acid complex-containing aqueous suspension.

### Comparative Example 80

The same operations as in Example 80, except for not adding poly-L-glutamic acid, were conducted.

### Test Example 6

In regard of Example 80 above, sodium chloride was added to each of the obtained anti-IgE monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-IgE monoclonal antibody was measured. Besides, in regard of Comparative Example 80, absorbance at a wavelength of 280 nm was measured as measurement of protein concentration, and, further, the activity of anti-IgE monoclonal antibody was measured. From the results of these measurements, the ratio of the protein concentration in Example 80 to the protein concentration in Comparative Example 80, and the ratio of the anti-IgE monoclonal antibody activity in Example 80 to the anti-IgE monoclonal antibody activity in Comparative Example 80 were determined. It was found that both the protein concentration ratio and the activity ratio rose to about 10 times, depicting that the anti-IgE monoclonal antibody can be concentrated without damaging the activity thereof. The results were set forth in Table 14.

TABLE 14

| | | Comparative | |
|---|---|---|---|
| | | Example 80 | Example 80 |
| Test | Polyamino acid/protein mass | 0 | 0.6 |
| Example | ratio | | |
| 6 | Protein concentration ratio | 1 | 10.1 |
| | Activity ratio | 1 | 10.2 |

### <L-Asparaginase>

### Example 81

In a 10 mM MOPS buffer (pH 7.0), L-asparaginase was prepared so as to attain a concentration of 0.5 mg/mL, and poly-L-lysine (MW: 4 kDa to 15 kDa) was added thereto in amounts of 0.025, 0.05, 0.10, 0.15, 0.3, 0.5, 0.7, and 1 part by mass based on 1 part by mass of L-asparaginase. The prepared liquids were centrifuged, and 90% of the total volume of the centrifuged liquid was removed as supernatant, to obtain L-asparaginase-poly-L-lysine complex-containing aqueous suspensions.

### Comparative Example 81

The same operations as in Example 81, except for not adding poly-L-lysine, were conducted.

### Test Example 7

In regard of Example 81 above, sodium chloride was added to the obtained L-asparaginase-poly-L-lysine complex-containing aqueous suspension so as to attain a concentration of 150 mM, then absorbance at a wavelength of 280 nm was measured as measurement of protein concentration, and, further, the activity of L-asparaginase was measured. Besides, in regard of Comparative Example 81, absorbance at a wavelength of 280 nm was measured as measurement of protein concentration, and, further, the activity of L-asparaginase was measured. From the results of these measurements, the ratio of the protein concentration in Example 81 to the protein concentration in Comparative Example 81, and the ratio of the L-asparaginase activity in Example 81 to the L-asparaginase activity in Comparative Example 81 were determined. It was found that both the protein concentration ratio and the activity ratio rose to about 10 times, depicting that the L-asparaginase can be concentrated without damaging the activity thereof. The results were set forth in Table 15.

TABLE 15

| | | Comparative | | | | | | | | |
|---|---|---|---|---|---|---|---|---|---|---|
| | | Example 81 | Example 81 | | | | | | | |
| Test | Polyamino | 0 | 0.025 | 0.05 | 0.1 | 0.15 | 0.3 | 0.5 | 0.7 | 1 |
| Example 7 | acid/protein mass | | | | | | | | | |
| | ratio | | | | | | | | | |
| | Protein concentration | 1 | 8.9 | 10.2 | 9.4 | 8.6 | 5.4 | 2.7 | 1.9 | 1.3 |
| | ratio | | | | | | | | | |
| | Activity ratio | 1 | 8.8 | 9.6 | 9.4 | 8.9 | 5.2 | 2.4 | 1.6 | 1.1 |

### Example 82

In a 10 mM MOPS buffer (pH 7.0), L-asparaginase was prepared so as to attain a concentration of 15 mg/mL, and poly-L-lysine (MW: 4 kDa to 15 kDa) was added thereto in amounts of 0.025, 0.05, 0.1, 0.15, and 0.3 part by mass based on 1 part by mass of L-asparaginase. Each of these prepared liquids was centrifuged, and 90% of the total volume of the centrifuged liquid was removed as supernatant, to obtain L-asparaginase-poly-L-lysine complex-containing aqueous suspensions.

### Comparative Example 82

The same operations as in Example 82, except for not adding poly-L-lysine, were conducted.

### Test Example 8

In regard of Example 82 above, sodium chloride was added to each of the obtained L-asparaginase-poly-L-lysine complex-containing aqueous suspension so as to attain a concentration of 150 mM, then absorbance at a wavelength of 280 nm was measured as measurement of protein concentration, and, further, the activity of L-asparaginase was measured. Besides, in regard of Comparative Example 82, absorbance at a wavelength of 280 nm was measured as measurement of protein concentration, and, further, the activity of L-asparaginase was measured. From these results, the ratio of the protein concentration in Example 82 to the protein concentration in Comparative Example 82, and the ratio of the L-asparaginase activity in Example 82 to the L-asparaginase activity in Comparative Example 82 were determined. It was found that both the protein concentration ratio and the activity ratio rose to about 10 times, depicting that the L-asparaginase can be concentrated without damaging the activity thereof. The results were set forth in Table 16.

TABLE 16

| | | Comparative | | | | | |
|---|---|---|---|---|---|---|---|
| | | Example 82 | Example 82 | | | | |
| Test | Polyamino | 0 | 0.025 | 0.05 | 0.1 | 0.15 | 0.3 |
| Example 8 | acid/protein | | | | | | |
| | mass ratio | | | | | | |
| | Protein | 1 | 11 | 11.1 | 9.5 | 8.3 | 3.8 |
| | concentra- | | | | | | |
| | tion ratio | | | | | | |
| | Activity ratio | 1 | 10.1 | 10.1 | 9.2 | 7.1 | 3.2 |

### <Anti-TNFα Monoclonal Antibody>

### Example 83

In a 10 mM MOPS buffer (pH 6.5), anti-TNFα monoclonal antibody was prepared so as to attain a concentration of 0.5 mg/mL, and poly-L-glutamic acid (MW: 3 kDa to 15 kDa) was added thereto in amounts of 0.05, 0.1, 0.15, 0.3, 0.5, and 1 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Comparative Example 83

The same operations as in Example 83, except for not adding poly-L-glutamic acid, were conducted.

### Test Example 9

In regard of Example 83 above, sodium chloride was added to the obtained anti-TNFα monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-TNFα monoclonal antibody was measured. Besides, in regard of Comparative Example 83, absorbance at a wavelength of 280 nm was measured as measurement of protein concentration, and, further, the activity of anti-TNFα monoclonal antibody was measured. From the results of these measurements, the ratio of the protein concentration in Example 83 to the protein concentration in Comparative Example 83, and the ratio of the anti-TNFα monoclonal antibody activity in Example 83 to the anti-TNFα monoclonal antibody activity in Comparative Example 83 were determined. It was found that both the protein concentration ratio and the activity ratio rose to about 10 times, depicting that the anti-TNFαmonoclonal antibody can be concentrated without damaging the activity thereof. The results were set forth in Table 17.

TABLE 17

| | | Comparative | | | | | |
|---|---|---|---|---|---|---|---|
| | | Example 83 | Example 83 | | | | |
| Test | Polyamino acid/ | 0 | 0.05 | 0.1 | 0.3 | 0.5 | 1 |
| Example 9 | protein mass ratio | | | | | | |
| | Protein | 1 | 10.2 | 10.2 | 9 | 7.7 | 6.4 |
| | concentration | | | | | | |
| | ratio | | | | | | |
| | Activity ratio | 1 | 10.3 | 10.3 | 8.5 | 7.1 | 5.8 |

### Example 84

In a 10 mM MOPS buffer (pH 6.5), anti-TNFα monoclonal antibody was prepared so as to attain a concentration of 15 mg/mL, and poly-L-glutamic acid (MW: 3 kDa to 15 kDa) was added thereto in an amount of 0.05 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody-poly-L-glutamic acid complex-containing aqueous suspensions.

### Comparative Example 84

The same operations as in Example 84, except for not adding poly-L-glutamic acid, were conducted.

### Test Example 10

In regard of Example 84 above, sodium chloride was added to the obtained anti-TNFα monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-TNFα monoclonal antibody was measured. Besides, in regard of Comparative Example 84, absorbance at a wavelength of 280 nm was measured as measurement of protein concentration, and, further, the activity of anti-TNFα monoclonal antibody was measured. From the results of these measurements, the ratio of the protein concentration in Example 84 to the protein concentration in Comparative Example 84, and the ratio of the anti-TNFα monoclonal antibody activity in Example 84 to the anti-TNFα monoclonal antibody activity in Comparative Example 84 were determined. It was found that both the protein concentration ratio and the activity ratio rose to about 10 times, depicting that the anti-TNFαmonoclonal antibody can be concentrated without damaging the activity thereof. The results were set forth in Table 18.

TABLE 18

| | | Comparative | |
|---|---|---|---|
| | | Example 84 | Example 84 |
| Test | Polyamino acid/protein mass | 0 | 0.05 |
| Example 10 | ratio | | |
| | Protein concentration ratio | 1 | 10.0 |
| | Activity ratio | 1 | 10.6 |

### Example 85

In a 10 mM citrate buffer (pH 4.7), anti-TNFα monoclonal antibody was prepared so as to attain a concentration of 0.5 mg/mL, and poly-L-glutamic acid (MW: 3 kDa to 15 kDa) was added thereto in an amount of 0.4 part by mass based on 1 part by mass of anti-TNFα monoclonal antibody. Each of these prepared liquids was centrifuged, and 90% of the total volume of the centrifuged liquid was removed as supernatant, to obtain each anti-TNFα monoclonal antibody-poly-L-glutamic acid complex-containing aqueous suspensions.

### Comparative Example 85

The same operations as in Example 85, except for not adding poly-L-glutamic acid, were conducted.

### Test Example 11

In regard of Example 85 above, sodium chloride was added to each of the obtained anti-TNFα monoclonal antibody-poly-L-glutamic acid complex-containing aqueous suspension so as to attain a concentration of 150 mM, then absorbance at a wavelength of 280 nm was measured as measurement of protein concentration, and, further, the activity of anti-TNFα monoclonal antibody was measured. Besides, in regard of Comparative Example 85, absorbance at a wavelength of 280 nm was measured as measurement of protein concentration, and, further, the activity of anti-TNFα monoclonal antibody was measured. From these results, the ratio of the protein concentration in Example 85 to the protein concentration in Comparative Example 85, and the ratio of the anti-TNFα monoclonal antibody activity in Example 85 to the anti-TNFα monoclonal antibody activity in Comparative Example 85 were determined. It was found that both the protein concentration ratio and the activity ratio rose to about 10 times, depicting that the anti-TNFα monoclonal antibody can be concentrated without damaging the activity thereof. The results were set forth in Table 19.

TABLE 19

| | | Comparative | Example |
|---|---|---|---|
| | | Example 85 | 85 |
| Test Example | Polyamino acid/protein mass | 0 | 0.4 |
| 11 | ratio | | |
| | Protein concentration ratio | 1 | 10.4 |
| | Activity ratio | 1 | 10.2 |

### <Anti-TNFα Monoclonal Antibody Fab Fragment>

### Example 86

In a 10 mM MOPS buffer (pH 6.5), anti-tumor necrosis factor (TNF) a monoclonal antibody Fab fragment (pI: 8.8, MW: 110 kDa) was prepared so as to attain a concentration of 1.16 mg/mL, and poly-L-glutamic acid (MW: 3 kDa to 15 kDa) was added thereto in amounts of 0.05, 0.1, and 0.3 part by mass based on 1 part by mass of anti-TNFα monoclonal antibody. Each of these prepared liquids was centrifuged, and 90% of the total volume of the centrifuged liquid was removed as supernatant, to obtain anti-TNFα monoclonal antibody Fab fragment-poly-L-glutamic acid complex-containing aqueous suspensions.

### Test Example 12

In regard of Example 86 above, sodium chloride was added to the obtained anti-TNFα monoclonal antibody Fab fragment-poly-L-glutamic acid complex-containing aqueous suspension so as to attain a concentration of 150 mM, then absorbance at a wavelength of 280 nm was measured as measurement of protein concentration, and, further, the activity of anti-TNFα monoclonal antibody Fab fragment was measured. Besides, in regard of Comparative Example 86, absorbance at a wavelength of 280 nm was measured as measurement of protein concentration, and, further, the activity of anti-TNFα monoclonal antibody Fab fragment was measured. From the results of these measurements, the ratio of the protein concentration in Example 86 to the protein concentration in Comparative Example 86, and the ratio of the anti-TNFα monoclonal antibody Fab fragment activity in Example 86 to the anti-TNFα monoclonal antibody Fab fragment activity in Comparative Example 86 were determined. It was found that both the protein concentration ratio and the activity ratio rose to about 10 times, depicting that the anti-TNFα monoclonal antibody Fab fragment can be concentrated without damaging the activity thereof. The results were set forth in Table 20.

TABLE 20

| | | Comparative | | | |
|---|---|---|---|---|---|
| | | Example 86 | Example 86 | | |
| Test | Polyamino acid/protein mass | 0 | 0.05 | 0.1 | 0.3 |
| Example | ratio | | | | |
| 12 | Protein concentration ratio | 1 | 9.8 | 8.6 | 4.5 |
| | Activity ratio | 1 | 10.2 | 9.3 | 5.2 |

### 5. Examples 84, 87, 88, and 88-2 in which Secondary Structure is Maintained Upon Concentration of Protein

### <Anti-TNFα Monoclonal Antibody>

### Measurement of CD Spectrum of Example 84

### Test Example 13

In regard of Example 84 above, sodium chloride was added to the obtained anti-TNFα monoclonal antibody-poly-L-glutamic acid complex-containing aqueous suspension so as to attain a concentration of 150 mM, and CD spectrum was measured. Besides, a liquid obtained by preparing anti-TNFα monoclonal antibody in a 10 mM MOPS buffer (pH 6.5) so as to attain a concentration of 15 mg/mL was made to be a control liquid (Comparative Example 84), which was put to measurement of CD. As a result, the CD spectra of both the liquids coincided with each other, verifying that the secondary structure of the protein was not changed but maintained upon concentration of the protein. The results were depicted in FIG. 6.

### Example 87

In a 10 mM MOPS buffer (pH 7.0), anti-TNFα monoclonal antibody was prepared so as to attain a concentration of 0.5 mg/mL, and poly-L-glutamic acid (MW: 3 kDa to 15 kDa) was added thereto in an amount of 0.04 part by mass based on 1 part by mass of anti-TNFα monoclonal antibody. Each of these prepared liquids was centrifuged, and 90% of the total volume of the centrifuged liquid was removed as supernatant, to obtain an anti-TNFα monoclonal antibody-poly-L-glutamic acid complex-containing aqueous suspension.

### Test Example 14

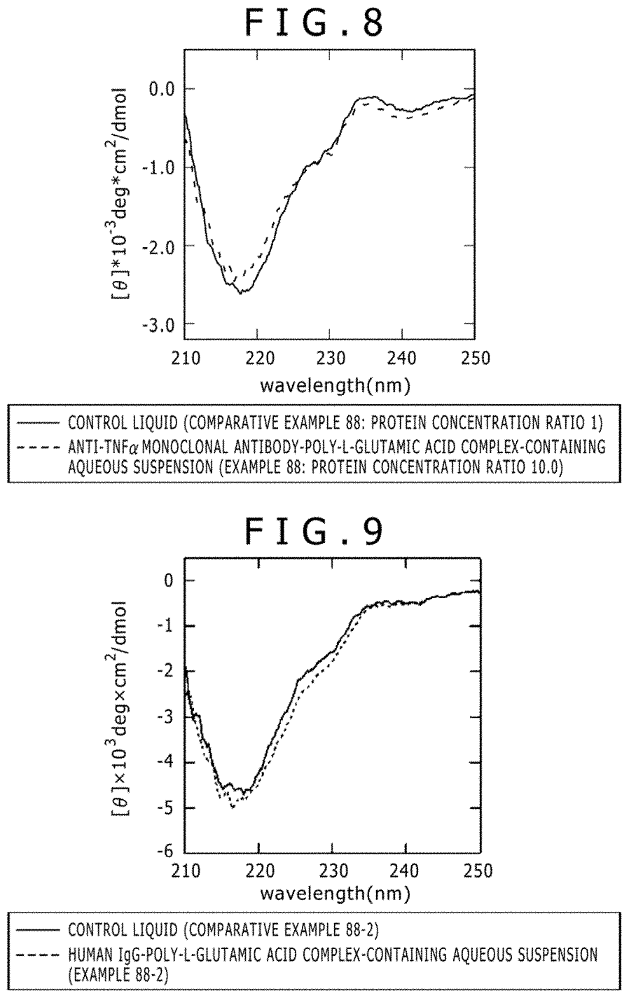

In regard of Example 87 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 5.0 mg/mL. Further, sodium chloride was added to the anti-TNFα monoclonal antibody-poly-L-glutamic acid complex-containing aqueous suspension so as to attain a concentration of 150 mM, and CD spectrum of the resulting liquid was measured. Besides, a liquid obtained by preparing anti-TNFα monoclonal antibody in a 10 mM MOPS buffer (pH 7.0) so as to attain a concentration of 0.5 mg/mL was made to be a control liquid (Comparative Example 87), which was put to measurement of CD spectrum. As a result, the CD spectra of both the liquids coincided with each other, verifying that the secondary structure of the protein was not changed but maintained upon concentration of the protein. The results were depicted in FIG. 7.

### Example 88

In a 10 mM citrate buffer (pH 4.7), anti-TNFα monoclonal antibody was prepared so as to attain a concentration of 0.5 mg/mL, and poly-L-glutamic acid (MW: 3 kDa to 15 kDa) was added thereto in an amount of 0.5 part by mass based on 1 part by mass of anti-TNFα monoclonal antibody. Each of these prepared liquids was centrifuged, and 90% of the total volume of the centrifuged liquid was removed as supernatant, to obtain an anti-TNFα monoclonal antibody-poly-L-glutamic acid complex-containing aqueous suspension.

### Test Example 15

In regard of Example 88 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 5.0 mg/mL. Further, sodium chloride was added to the anti-TNFα monoclonal antibody-poly-L-glutamic acid complex-containing aqueous suspension so as to attain a concentration of 150 mM, and CD spectrum was measured. Besides, a liquid obtained by preparing anti-TNFα monoclonal antibody in a 10 mM citrate buffer (pH 5.2) so as to attain a concentration of 0.5 mg/mL was made to be a control liquid (Comparative Example 88), which was put to measurement of CD spectrum. As a result, the CD spectra of both of the liquids coincided with each other, verifying that the secondary structure of the protein was not changed but maintained upon concentration of the protein. The results were depicted in FIG. 8.

### <Human IgG>

### Example 88-2

In a 10 mM citrate buffer (pH 5.0), human IgG was prepared so as to attain a concentration of 30 mg/mL, and poly-L-glutamic acid (MW: 3 kDa to 15 kDa) was added thereto in an amount of 0.05 part by mass based on 1 part by mass of human IgG. Each of these prepared liquids was centrifuged, and 90% of the total volume of the centrifuged liquid was removed as supernatant, to obtain a human IgG-poly-L-glutamic acid complex-containing aqueous suspension.

### Test Example 15-2

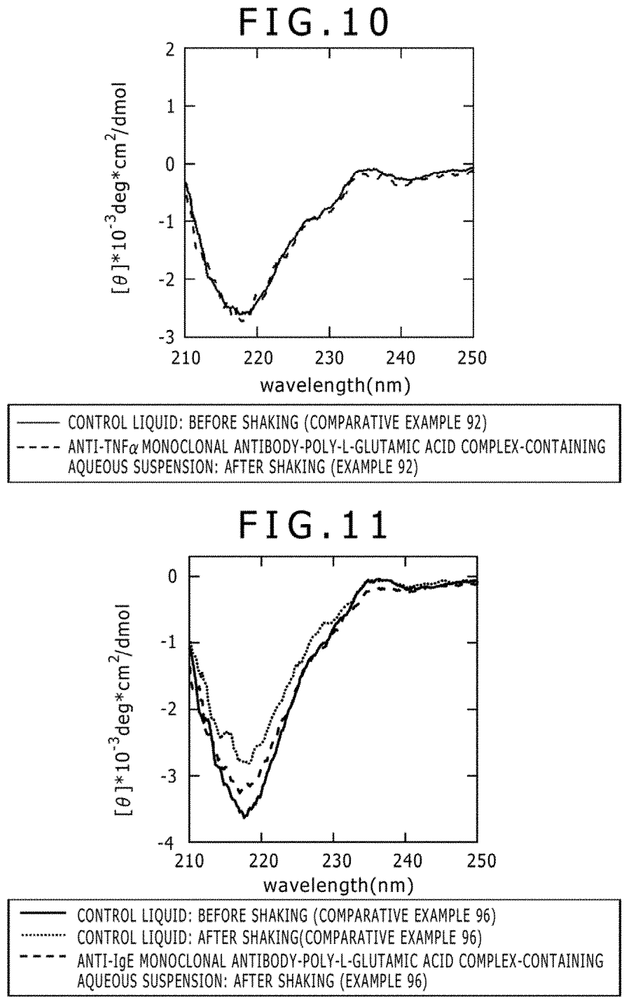

In regard of Example 88-2 above, part of the obtained human IgG-poly-L-glutamic acid complex-containing aqueous suspension was sampled, sodium chloride was added thereto so as to attain a concentration of 600 mM, absorbance at a wavelength of 280 nm was measured as measurement of protein concentration, and, further, CD spectrum was measured. Besides, a liquid obtained by preparing human IgG in a 10 mM citrate buffer (pH 5.0) so as to attain a concentration of 0.5 mg/mL was made to be a control liquid (Comparative Example 87), which was put to measurement of CD spectrum. As a result, the CD spectra of both of the liquids coincided with each other, verifying that the secondary structure of the protein was not changed but maintained upon concentration of the protein. The results were depicted in FIG. 9.

### 6. Examples 89 to 96 which Depict Stabilizing Effect Against Shaking Stress

### <L-Asparaginase>

### Example 89

In a 10 mM MOPS buffer (pH 7.0), L-asparaginase was prepared so as to attain a concentration of 0.5 mg/mL, and poly-L-lysine (MW: 4 kDa to 15 kDa) was added thereto in an amount of 0.05 part by mass based on 1 part by mass of L-asparaginase. The thus prepared liquid was centrifuged, and 90% of the total volume of the centrifuged liquid was removed as supernatant, to obtain an L-asparaginase-poly-L-lysine complex-containing aqueous suspension.

### Test Example 16

In regard of Example 89 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 5.0 mg/mL. The rest of the L-asparaginase-poly-L-lysine complex-containing aqueous suspension was filled into a polypropylene-made disposable syringe, which was placed on a shaker (Bioshaker V⋅BR-36), and shaking at 500 rpm was conducted at room temperature for 60 hours. Sodium chloride was added to the L-asparaginase-poly-L-lysine complex-containing aqueous suspension before the shaking and to that after the shaking so as to attain a concentration of 150 mM, and the activity of L-asparaginase was measured. Besides, a liquid obtained by preparing L-asparaginase in a 10 mM MOPS buffer (pH 7.0) so as to attain a concentration of 5.0 mg/mL was made to be a control liquid (Comparative Example 89), which was filled into a syringe, and subjected to shaking simultaneously with the aqueous suspension. For the control liquid before the shaking and that after the shaking, the activity of L-asparaginase was measured. From the results of these measurements, the retention rate of L-asparaginase activity after shaking based on the L-asparaginase activity before the shaking was determined, for the L-asparaginase-poly-L-lysine complex-containing aqueous suspension (Example 89) and the control liquid (Comparative Example 89). For the control liquid, a lowering in the activity was observed. For the L-asparaginase-poly-L-lysine complex-containing aqueous suspension, on the other hand, a lowering in the activity was not observed. Thus, a stabilizing effect against shaking stress was depicted as to the protein-polyamino acid complex-containing aqueous suspension. The results were set forth in Table 21.

TABLE 21

| | | | Protein- |
|---|---|---|---|
| | | | polyamino acid |
| | | Control liquid | complex-containing |
| | | (Comparative | aqueous suspension |
| | | Example 89) | (Example 89) |
| Test | Retention rate of | 64.8 | 97.8 |
| Example | activity after shaking | | |
| 16 | based on activity | | |
| | before shaking (%) | | |

### Example 90

The same operations as in Example 81 were conducted, except that L-asparaginase was prepared in a 10 mM MOPS buffer (pH 7.0) so as to attain a concentration of 1.0 mg/mL.

### Test Example 17

In regard of Example 90 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 10.0 mg/mL. The rest of the L-asparaginase-poly-L-lysine complex-containing aqueous suspension was filled into a polypropylene-made disposable syringe, which was placed on a shaker (Bioshaker V⋅BR-36), and shaking at 500 rpm was conducted at room temperature for 60 hours. Sodium chloride was added to the L-asparaginase-poly-L-lysine complex-containing aqueous suspension before the shaking and to that after the shaking so as to attain a concentration of 150 mM, and the activity of L-asparaginase was measured. Besides, a liquid obtained by preparing L-asparaginase in a 10 mM MOPS buffer (pH 7.0) so as to attain a concentration of 10.0 mg/mL was made to be a control liquid (Comparative Example 90), which was filled into a syringe, and subjected to shaking simultaneously with the aqueous suspension. For the control liquid before the shaking and that after the shaking, the activity of L-asparaginase was measured. From the results of these measurements, the retention rate of L-asparaginase activity after shaking based on the L-asparaginase activity before the shaking was determined, for the L-asparaginase-poly-L-lysine complex-containing aqueous suspension and the control liquid. For the control liquid, a lowering in the activity was observed. For the L-asparaginase-poly-L-lysine complex-containing aqueous suspension, on the other hand, a lowering in the activity was not observed. Thus, a stabilizing effect against shaking stress was depicted as to the protein-polyamino acid complex-containing aqueous suspension The results were set forth in Table 22.

TABLE 22

| | | | Protein- |
|---|---|---|---|
| | | | polyamino acid |
| | | Control liquid | complex-containing |
| | | (Comparative | aqueous suspension |
| | | Example 90) | (Example 90) |
| Test | Retention rate of | 73.6 | 102.6 |
| Example | activity after shaking | | |
| 17 | based on activity | | |
| | before shaking (%) | | |

### <Anti-EGFR Monoclonal Antibody>

### Example 91

In a 10 mM MOPS buffer (pH 5.0), anti-EGFR monoclonal antibody was prepared so as to attain a concentration of 13.3 mg/mL, and poly-L-glutamic acid (MW: 3 kDa to 15 kDa) was added thereto in an amount of 0.07 part by mass based on 1 part by mass of anti-EGFR monoclonal antibody. The thus prepared liquid was centrifuged, and 90% of the total volume of the centrifuged liquid was removed as supernatant, to obtain an anti-EGFR monoclonal antibody-poly-L-glutamic acid complex-containing aqueous suspension.

### Test Example 18

In regard of Example 91 above, part of the obtained anti-EGFR monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 78.5 mg/mL. The rest of the anti-EGFR monoclonal antibody-poly-L-glutamic acid complex-containing aqueous suspension was filled into a polypropylene-made disposable syringe, which was placed on a shaker (Bioshaker V⋅BR-36), and shaking at 500 rpm was conducted at room temperature for 90 hours. Sodium chloride was added to the anti-EGFR monoclonal antibody-poly-L-glutamic acid complex-containing aqueous suspension before the shaking and to that after the shaking so as to attain a concentration of 150 mM, the resulting aqueous suspensions were centrifuged at 10,000 g for 10 minutes, and supernatants were put to measurement of absorbance at a wavelength of 280 nm as measurement of protein concentration. Besides, a liquid obtained by preparing anti-EGFR in a 10 mM MOPS buffer (pH 5.0) so as to attain a concentration of 78.5 mg/mL was made to be a control liquid (Comparative Example 91), which was filled into a syringe, and subjected to shaking simultaneously with the aqueous suspension. Sodium chloride was added to the control liquid (Comparative Example 91) before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, the retention rate of protein content after the shaking based on the protein content before the shaking was determined, for the anti-EGFR monoclonal antibody-poly-L-glutamic acid complex-containing aqueous suspension and the control liquid. For the control liquid, a lowering in the retention rate was observed. For the anti-EGFR monoclonal antibody-poly-L-glutamic acid complex-containing aqueous suspension, on the other hand, a lowering in the retention rate was not observed. Thus, a stabilizing effect against shaking stress was depicted as to the protein-polyamino acid complex-containing aqueous suspension. The results were set forth in Table 23.

TABLE 23

| | | Control liquid | Protein-polyamino acid | |
|---|---|---|---|---|
| | | (Comparative | complex-containing aqueous | Significant test |
| | | Example 91) | suspension (Example 91) | (t-test) |
| Test Example | Retention rate of protein content | 86.6 | 105.3 | 0.0008 |
| 18 | after shaking based on protein | | | |
| | content before shaking (%) | | | |

### <Anti-TNFα Monoclonal Antibody>

### Example 92

In a 10 mM MOPS buffer (pH 7.0), anti-TNFα monoclonal antibody was prepared so as to attain a concentration of 0.5 mg/mL, and poly-L-glutamic acid (MW: 3 kDa to 15 kDa) was added thereto in an amount of 0.04 part by mass based on 1 part by mass of anti-TNFα monoclonal antibody. The thus prepared liquid was centrifuged, and 90% of the total volume of the centrifuged liquid was removed as supernatant, to obtain an anti-TNFα monoclonal antibody-poly-L-glutamic acid complex-containing aqueous suspension.

### Test Example 19

In regard of Example 92 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 5.0 mg/mL. To the rest of the anti-TNFα monoclonal antibody-poly-L-glutamic acid complex-containing aqueous suspension, a ninefold amount of a 10 mM MOPS buffer (pH 7.0) was added to attain a protein concentration of 0.5 mg/mL. The thus diluted aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-TNFα monoclonal antibody-poly-L-glutamic acid complex-containing aqueous suspension before the shaking and to that after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration.

Besides, a liquid obtained by preparing anti-TNFα monoclonal antibody in a 10 mM MOPS buffer (pH 7.0) so as to attain a concentration of 0.5 mg/mL was made to be a control liquid (Comparative Example 92), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, the retention rate of protein content after the shaking based on the protein content before the shaking was determined, for the anti-TNFα monoclonal antibody-poly-L-glutamic acid complex-containing aqueous suspension and the control liquid. For the control liquid, a conspicuous lowering in the retention rate was observed. For the anti-TNFα monoclonal antibody-poly-L-glutamic acid complex-containing aqueous suspension, on the other hand, a lowering in the retention rate was not observed. Thus, a stabilizing effect against shaking stress was depicted as to the protein-polyamino acid complex-containing aqueous suspension. The results were set forth in Table 24.

TABLE 24

| | | | Protein- |
|---|---|---|---|
| | | | polyamino acid |
| | | Control liquid | complex-containing |
| | | (Comparative | aqueous suspension |
| | | Example 92) | (Example 92) |
| Test | Retention rate of | 6 | 70.0 |
| Example | protein content after | | |
| 19 | shaking based on | | |
| | protein content before | | |
| | shaking (%) | | |

### Example 93

In a 10 mM MOPS buffer (pH 6.5), anti-TNFα monoclonal antibody was prepared so as to attain a concentration of 0.5 mg/mL, and poly-L-glutamic acid (MW: 3 kDa to 15 kDa) was added thereto in an amount of 0.05 part by mass based on 1 part by mass of anti-TNFα monoclonal antibody. The thus prepared liquid was centrifuged, and 90% of the total volume of the centrifuged liquid was removed as supernatant, to obtain an anti-TNFα monoclonal antibody-poly-L-glutamic acid complex-containing aqueous suspension.

### Test Example 20

In regard of Example 93 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 5.0 mg/mL. To the rest of the anti-TNFα monoclonal antibody-poly-L-glutamic acid complex-containing aqueous suspension, a ninefold amount of a 10 mM MOPS buffer (pH 6.5) was added to attain a protein concentration of 0.5 mg/mL. The thus diluted aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-TNFα monoclonal antibody-poly-L-glutamic acid complex-containing aqueous suspension before the shaking and to that after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration.

Besides, a liquid obtained by preparing anti-TNFα monoclonal antibody in a 10 mM MOPS buffer (pH 6.5) so as to attain a concentration of 0.5 mg/mL was made to be a control liquid (Comparative Example 93), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, the retention rate of protein content after the shaking based on the protein content before the shaking was determined, for the anti-TNFα monoclonal antibody-poly-L-glutamic acid complex-containing aqueous suspension and the control liquid. For the control liquid, a conspicuous lowering in the retention rate was observed. For the anti-TNFα monoclonal antibody-poly-L-glutamic acid complex-containing aqueous suspension, on the other hand, a lowering in the retention rate was not observed. Thus, a stabilizing effect against shaking stress was depicted as to the protein-polyamino acid complex-containing aqueous suspension. The results were set forth in Table 25.

TABLE 25

| | | | Protein- |
|---|---|---|---|
| | | | polyamino acid |
| | | Control liquid | complex-containing |
| | | (Comparative | aqueous suspension |
| | | Example 93) | (Example 93) |
| Test | Retention rate of | 10.6 | 94.9 |
| Example | protein content after | | |
| 20 | shaking based on | | |
| | protein content before | | |
| | shaking (%) | | |

### Test Example 21

In regard of Example 93 above, sodium chloride was added to the obtained anti-TNFα monoclonal antibody-poly-L-glutamic acid complex-containing aqueous suspension so as to attain a concentration of 150 mM, and the activity of anti-TNFα monoclonal antibody was measured. Besides, in regard of Comparative Example 93, the activity of anti-TNFα monoclonal antibody was measured. From the results of these measurements, the retention rate of the activity after shaking based on the activity before shaking was determined for the anti-TNFα monoclonal antibody-poly-L-glutamic acid complex-containing aqueous suspension and the control liquid. For the control liquid, a conspicuous lowering in the activity retention rate was observed. For the anti-TNFα monoclonal antibody-poly-L-glutamic acid complex-containing aqueous suspension, on the other hand, a lowering in the activity retention rate was restrained significantly. Thus, a stabilizing effect against shaking stress was depicted as to the protein-polyamino acid complex-containing aqueous suspension. The results were set forth in Table 26.

TABLE 26

| | | | Protein- |
|---|---|---|---|
| | | | polyamino acid |
| | | Control liquid | complex-containing |
| | | (Comparative | aqueous suspension |
| | | Example 93) | (Example 93) |
| Test | Retention rate of | 37.5 | 96.8 |
| Example | protein content after | | |
| 21 | shaking based on | | |
| | protein content before | | |
| | shaking (%) | | |

### Example 94

In a 10 mM MOPS buffer (pH 6.5), anti-TNFα monoclonal antibody was prepared so as to attain a concentration of 5 mg/mL, and poly-L-glutamic acid (MW: 3 kDa to 15 kDa) was added thereto in an amount of 0.05 part by mass based on 1 part by mass of anti-TNFα monoclonal antibody. The thus prepared liquid was centrifuged, and 90% of the total volume of the centrifuged liquid was removed as supernatant, to obtain an anti-TNFα monoclonal antibody-poly-L-glutamic acid complex-containing aqueous suspension.

### Test Example 22

In regard of Example 94 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 50.0 mg/mL. To the rest of the anti-TNFα monoclonal antibody-poly-L-glutamic acid complex-containing aqueous suspension, a ninefold amount of a 10 mM MOPS buffer (pH 6.5) was added to attain a protein concentration of 5.0 mg/mL. The thus diluted aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-TNFα monoclonal antibody-poly-L-glutamic acid complex-containing aqueous suspension before the shaking and to that after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration.

Besides, a liquid obtained by preparing anti-TNFα monoclonal antibody in a 10 mM MOPS buffer (pH 6.5) so as to attain a concentration of 5.0 mg/mL was made to be a control liquid (Comparative Example 94), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, the retention rate of protein content after the shaking based on the protein content before the shaking was determined, for the anti-TNFα monoclonal antibody-poly-L-glutamic acid complex-containing aqueous suspension and the control liquid. For the control liquid, a lowering in the retention rate was observed. For the anti-TNFα monoclonal antibody-poly-L-glutamic acid complex-containing aqueous suspension, on the other hand, a lowering in the retention rate was not observed. Thus, a stabilizing effect against shaking stress was depicted as to the protein-polyamino acid complex-containing aqueous suspension. The results were set forth in Table 27.

TABLE 27

| | | Control liquid | Protein-polyamino acid complex- | |
|---|---|---|---|---|
| | | (Comparative | containing aqueous suspension | Significant test |
| | | Example 94) | (Example 94) | (t-test) |
| Test | Retention rate of protein content | 75.2 | 103.5 | 0.0006 |
| Example 22 | after shaking based on protein | | | |
| | content before shaking (%) | | | |

### Example 95

The same operations as in Example 94 were conducted, except that the amount of the solution prepared was increased, specifically that while 90% of the total volume of the centrifuged liquid was removed as supernatant to obtain 0.06 mL of the anti-TNFα monoclonal antibody-poly-L-glutamic acid complex-containing aqueous suspension in Example 94, the amount of the liquid prepared before the centrifugation was increased to 6/8 times that in Example 94 and, thereby, 0.08 mL of an aqueous suspension was obtained.

### Test Example 23

In regard of Example 95 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 50.0 mg/mL. To the rest of the anti-TNFα monoclonal antibody-poly-L-glutamic acid complex-containing aqueous suspension, a ninefold amount of a 10 mM MOPS buffer (pH 6.5) was added to attain a protein concentration of 5.0 mg/mL. The thus diluted aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-TNFα monoclonal antibody-poly-L-glutamic acid complex-containing aqueous suspension before the shaking and to that after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration.

Besides, a liquid obtained by preparing anti-TNFα monoclonal antibody in a 10 mM MOPS buffer (pH 6.5) so as to attain a concentration of 5.0 mg/mL was made to be a control liquid (Comparative Example 95), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, the retention rate of protein content after the shaking based on the protein content before the shaking was determined, for the anti-TNFα monoclonal antibody-poly-L-glutamic acid complex-containing aqueous suspension and the control liquid. For the control liquid, a lowering in the retention rate was observed. For the anti-TNFα monoclonal antibody-poly-L-glutamic acid complex-containing aqueous suspension, on the other hand, a lowering in the retention rate was not observed. Thus, a stabilizing effect against shaking stress was depicted as to the protein-polyamino acid complex-containing aqueous suspension. The results were set forth in Table 28.

TABLE 28

| | | | Protein- |
|---|---|---|---|
| | | | polyamino acid |
| | | Control liquid | complex-containing |
| | | (Comparative | aqueous suspension |
| | | Example 95) | (Example 95) |
| Test | Retention rate of | 52.1 | 98.3 |
| Example | protein content after | | |
| 23 | shaking based on | | |
| | protein content before | | |
| | shaking (%) | | |

### Test Example 24

In regard of the same sample as the sample which was subjected to measurement of protein concentration in the supernatant in Test Example 23 above, the activity of anti-TNFα monoclonal antibody was measured. From the results of these measurements, the retention rate of the activity after shaking based on the activity before shaking was determined for the anti-TNFα monoclonal antibody-poly-L-glutamic acid complex-containing aqueous suspension and the control liquid. For the control liquid, a conspicuous lowering in the activity retention rate was observed. For the anti-TNFα monoclonal antibody-poly-L-glutamic acid complex-containing aqueous suspension, on the other hand, a lowering in the activity retention rate was restrained significantly. Thus, a stabilizing effect against shaking stress was depicted as to the protein-polyamino acid complex-containing aqueous suspension. The results were set forth in Table 29.

TABLE 29

| | | | Protein- |
|---|---|---|---|
| | | | polyamino acid |
| | | Control liquid | complex-containing |
| | | (Comparative | aqueous suspension |
| | | Example 95) | (Example 95) |
| Test | Retention rate of | 53.8 | 102.7 |
| Example | protein content after | | |
| 24 | shaking based on | | |
| | protein content before | | |
| | shaking (%) | | |

### <Anti-IgE Monoclonal Antibody>

### Example 96

In a 10 mM citrate buffer (pH 5.4), anti-IgE monoclonal antibody was prepared so as to attain a concentration of 0.5 mg/mL, and poly-L-glutamic acid (MW: 3 kDa to 15 kDa) was added thereto in an amount of 0.1 part by mass based on 1 part by mass of anti-IgE monoclonal antibody. The thus prepared liquid was centrifuged, and 90% of the total volume of the centrifuged liquid was removed as supernatant, to obtain an anti-IgE monoclonal antibody-poly-L-glutamic acid complex-containing aqueous suspension.

### Test Example 25

In regard of Example 96 above, part of the obtained anti-IgE monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 5.0 mg/mL. To the rest of the anti-IgE monoclonal antibody-poly-L-glutamic acid complex-containing aqueous suspension, a ninefold amount of a 10 mM citrate buffer (pH 5.4) was added to attain a protein concentration of 0.5 mg/mL. The thus diluted aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-IgE monoclonal antibody-poly-L-glutamic acid complex-containing aqueous suspension before the shaking and to that after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration.

Besides, a liquid obtained by preparing anti-IgE monoclonal antibody in a 10 mM citrate buffer (pH 5.5) so as to attain a concentration of 0.5 mg/mL was made to be a control liquid (Comparative Example 96), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, the retention rate of protein content after the shaking based on the protein content before the shaking was determined, for the anti-IgE monoclonal antibody-poly-L-glutamic acid complex-containing aqueous suspension and the control liquid. For the control liquid, a conspicuous lowering in the retention rate was observed. For the anti-IgE monoclonal antibody-poly-L-glutamic acid complex-containing aqueous suspension, on the other hand, a lowering in the retention rate was not observed. Thus, a stabilizing effect against shaking stress was depicted as to the protein-polyamino acid complex-containing aqueous suspension. The results were set forth in Table 30.

TABLE 30

| | | | Protein- |
|---|---|---|---|
| | | | polyamino acid |
| | | Control liquid | complex-containing |
| | | (Comparative | aqueous suspension |
| | | Example 96) | (Example 96) |
| Test | Retention rate of | 9 | 97 |
| Example | protein content after | | |
| 25 | shaking based on | | |
| | protein content before | | |
| | shaking (%) | | |

### 7. Examples 97 and 98 in which Secondary Structure is Maintained Against Shaking Stress

### <Anti-TNFα Monoclonal Antibody>

### Test Example 26

In regard of the anti-TNFα monoclonal antibody-poly-L-glutamic acid complex-containing aqueous suspension (Example 92) obtained in Test Example 19 above, CD spectrum was measured after the shaking. On the other hand, in regard of the control liquid (Comparative Example 92) obtained in Test Example 19, CD spectrum was measured before the shaking. As a result, the CD spectrum in the case where the shaking stress was exerted on the anti-TNFα monoclonal antibody-poly-L-glutamic acid complex-containing aqueous suspension and the CD spectrum in the case where the shaking stress was not exerted coincided with each other. Thus it was verified that the secondary structure is maintained even when shaking stress is exerted on the anti-TNFα monoclonal antibody-poly-L-glutamic acid complex-containing aqueous suspension. The results are depicted in FIG. 10.

### <Anti-IgE Monoclonal Antibody>

### Test Example 27

In regard of the anti-IgE monoclonal antibody-poly-L-glutamic acid complex-containing aqueous suspension (Example 96) obtained in Test Example 25 above, CD spectrum was measured after the shaking. On the other hand, in regard of the control liquid (Comparative Example 96) obtained in Test Example 25, CD spectrum was measured before the shaking and after the shaking. As a result, the CD spectrum in the case where shaking stress was exerted on the control liquid and the CD spectrum in the case where the shaking stress was not exerted did not coincide with each other, and a change in secondary structure is generated. On the other hand, the CD spectrum in the case where shaking stress was exerted on the anti-IgE monoclonal antibody-poly-L-glutamic acid complex-containing aqueous suspension and the CD spectrum in the case where the shaking stress was not exerted coincided with each other. Thus, it was verified that the secondary structure is maintained even when shaking stress is exerted on the anti-IgE monoclonal antibody-poly-L-glutamic acid complex-containing aqueous suspension. The results were depicted in FIG. 11.

### 8. Example 99 which Depicts Enhancement of Fluidity

### <Anti-IgE Monoclonal Antibody>

### Example 99

In a 10 mM MOPS buffer (pH 5.5), anti-IgE monoclonal antibody was prepared so as to attain each of concentrations of 6.0 mg/mL, 8.0 mg/mL, and 10.0 mg/mL, and poly-L-glutamic acid (MW: 3 kDa to 15 kDa) was added individually thereto in an amount of 0.08 part by mass based on 1 part by mass of anti-IgE monoclonal antibody. The thus prepared liquids were centrifuged, and 90% of the total volume of the centrifuged liquid was removed as supernatant, to obtain each anti-IgE monoclonal antibody-poly-L-glutamic acid complex-containing aqueous suspension.

### Test Example 28

In regard of Example 99 above, part of each of the obtained anti-IgE monoclonal antibody-poly-L-glutamic acid complex-containing aqueous suspensions was sampled, sodium chloride was added thereto so as to attain a concentration of 150 mM, and absorbance at a wavelength of 280 nm was measured as measurements of protein concentration, which were 60.0 mg/mL, 80.0 mg/mL, and 100.0 mg/mL. In regard of the rest of each anti-IgE monoclonal antibody-poly-L-glutamic acid complex-containing aqueous suspension, viscosity was measured by a differential pressure type viscometer. Besides, liquids obtained by preparing anti-IgE monoclonal antibody in a 10 mM MOPS buffer (pH 5.5) so as to attain concentrations of 60.0 mg/mL, 80.0 mg/mL, and 100.0 mg/mL were made to be control liquids (Comparative Example 99), and viscosity was measured by a differential pressure type viscometer. From the results of these measurements, it was verified that the anti-IgE monoclonal antibody-poly-L-glutamic acid complex-containing aqueous suspensions are lower in viscosity and higher in fluidity than the control liquids. The results were set forth in Table 31.

TABLE 31

| | | Viscosity (mPa/s) | |
|---|---|---|---|
| | | | Protein-polyamino acid |
| | Protein | Control liquid | complex-containing |
| | concentration | (Comparative | aqueous suspension |
| | (mg/mL) | Example 99) | (Example 99) |
| Test | 60 | 4.62 | 2.74 |
| Example | 80 | 9.6 | 5.2 |
| 28 | 100 | 21.87 | 10.96 |

### 9. Examples 100 and 101 which Depict Stabilizing Effect Against Oxidation Stress

<L-Asparaginase; Protein Concentration 40.0 mg/mL>

### Example 100

In a 10 mM MOPS buffer (pH 7.0), L-asparaginase was prepared so as to attain a concentration of 18 mg/mL, and poly-L-lysine (MW: 4 kDa to 15 kDa) was added thereto in an amount of 0.05 part by mass based on 1 part by mass of L-asparaginase. The thus prepared liquid was centrifuged, and 90% of the total volume of the centrifuged liquid was removed as supernatant, to obtain an L-asparaginase-poly-L-lysine complex-containing aqueous suspension.

### Test Example 29

In regard of Example 100 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 90.0 mg/mL. To the rest of the L-asparaginase-poly-L-lysine complex-containing aqueous suspension, a 1.25-fold amount of a 0.18% hydrogen peroxide/10 mM MOPS buffer (pH 7.0) was added, to obtain 40.0 mg/mL L-asparaginase-poly-L-lysine complex-containing aqueous suspension/0.1% hydrogen peroxide. This liquid was maintained at 37° C. for respective periods of time of two hours, four hours, and six hours. Besides, a 1.25-fold amount of a 10 mM MOPS buffer (pH 7.0) was added in place of the 0.18% by weight hydrogen peroxide/10 mM MOPS buffer (pH 7.0), to obtain a 40.0 mg/mL L-asparaginase-poly-L-lysine complex-containing aqueous suspension. These liquids were kept at 37° C. for periods of time of two hours, four hours, and six hours, then sodium chloride was added thereto so as to attain a concentration of 150 mM, and the activity of L-asparaginase was measured. Besides, a liquid obtained by preparing L-asparaginase in a 10 mM MOPS buffer (pH 7.0) so as to attain a concentration of 90.0 mg/mL was made to be a control liquid, to which a 1.25-fold amount of 0.18% by weight hydrogen peroxide/10 mM MOPS buffer (pH 7.0) was added, to obtain 40.0 mg/mL L-asparaginase/0.1% hydrogen peroxide. Further, a 1.25-fold amount of a 10 mM MOPS buffer (pH 7.0) was added in place of the 0.18% by weight hydrogen peroxide/10 mM MOPS buffer (pH 7.0), to obtain 40.0 mg/mL L-asparaginase. These control liquids were kept at 37° C. simultaneously. After the liquids were kept at 37° C. for two hours, four hours, and six hours, sodium chloride was added thereto so as to attain a concentration of 150 mM, and the activity of L-asparaginase was measured. From the results of these measurements, the retention rate of L-asparaginase activity in the presence of hydrogen peroxide based on L-asparaginase activity in the absence of hydrogen peroxide was determined, for the L-asparaginase-poly-L-lysine complex-containing aqueous suspensions and the control liquids. For the control liquids, a conspicuous lowering in the activity was observed. For the L-asparaginase-poly-L-lysine complex-containing aqueous suspension, on the other hand, the lowering in the activity was smaller. Thus, a stabilizing effect against oxidation stress was verified as to the protein-polyamino acid complex-containing aqueous suspension. The results were set forth in Table 32.

<L-Asparaginase; Protein Concentration 80.0 mg/mL>

### Test Example 30

In regard of Example 100 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 90.0 mg/mL. To the rest of the L-asparaginase-poly-L-lysine complex-containing aqueous suspension, a 0.125-fold amount of a 0.9% by weight hydrogen peroxide/10 mM MOPS buffer (pH 7.0) was added, to obtain 80.0 mg/mL L-asparaginase-poly-L-lysine complex-containing aqueous suspension/0.1% hydrogen peroxide. This liquid was kept at 37° C. for periods of time of two hours, four hours, and six hours. Besides, a 0.125-fold amount of a 10 mM MOPS buffer (pH 7.0) was added in place of the 0.9% by weight hydrogen peroxide/10 mM MOPS buffer (pH 7.0), to obtain a 80.0 mg/mL L-asparaginase-poly-L-lysine complex-containing aqueous suspension. These liquids were kept at 37° C. for periods of time of two hours, four hours, and six hours, then sodium chloride was added thereto so as to attain a concentration of 150 mM, and the activity of L-asparaginase was measured. Besides, a liquid obtained by preparing L-asparaginase in a 10 mM MOPS buffer (pH 7.0) so as to attain a concentration of 90.0 mg/mL was made to be a control liquid, to which a 0.125-fold amount of 0.9% by weight hydrogen peroxide/10 mM MOPS buffer (pH 7.0) was added, to obtain 80.0 mg/mL L-asparaginase/0.1% hydrogen peroxide. Further, a 0.125-fold amount of a 10 mM MOPS buffer (pH 7.0) was added in place of the 0.9% by weight hydrogen peroxide/10 mM MOPS buffer (pH 7.0), to obtain 80.0 mg/mL L-asparaginase. These control liquids were kept at 37° C. simultaneously. After the liquids were kept at 37° C. for two hours, four hours, and six hours, sodium chloride was added thereto so as to attain a concentration of 150 mM, and the activity of L-asparaginase was measured. From the results of these measurements, the retention rate of L-asparaginase activity in the presence of hydrogen peroxide based on L-asparaginase activity in the absence of hydrogen peroxide was determined, for the L-asparaginase-poly-L-lysine complex-containing aqueous suspensions and the control liquids. For the control liquids, a conspicuous lowering in the activity was observed. For the L-asparaginase-poly-L-lysine complex-containing aqueous suspensions, on the other hand, the lowering in the activity was smaller. Thus, a stabilizing effect against oxidation stress was verified as to the protein-polyamino acid complex-containing aqueous suspension. The results were set forth in Table 32.

TABLE 32

| | | | | Protein-polyamino acid |
|---|---|---|---|---|
| | | Hydrogen peroxide | Control liquid | complex-containing |
| | | treatment time | (Comparative | aqueous suspension |
| | | (hr) | Example 100) | (Example 100) |
| Test Example 29 | Retention rate of activity | 2 | 89.1 | 95.7 |
| Protein concentration | in the presence of 0.1% | 4 | 76 | 89.7 |
| 40.0 mg/ml | hydrogen peroxide based | 6 | 67.3 | 83 |
| | on activity in the absence | | | |
| | of hydrogen peroxide (%) | | | |
| Test Example 30 | Retention rate of activity | 2 | 87.2 | 95 |
| Protein concentration | in the presence of 0.1% | 4 | 78.9 | 87.2 |
| 80.0 mg/ml | hydrogen peroxide based on | 6 | 68.7 | 82.5 |
| | activity in the absence of | | | |
| | hydrogen peroxide (%) | | | |

### <Anti-IgE Monoclonal Antibody>

### Example 101

In a 10 mM MOPS buffer (pH 5.5), anti-IgE monoclonal antibody was prepared so as to attain a concentration of 6.0 mg/mL, and poly-L-glutamic acid (MW: 3 kDa to 15 kDa) was added thereto in an amount of 0.1 part by mass based on 1 part by mass of anti-IgE monoclonal antibody. The thus prepared liquid was centrifuged, and 90% of the total volume of the centrifuged liquid was removed as supernatant, to obtain an anti-IgE monoclonal antibody-poly-L-glutamic acid complex-containing aqueous suspension.

### Test Example 31

In regard of Example 101 above, part of the obtained anti-IgE monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 60.0 mg/mL. To the rest of the anti-IgE monoclonal antibody-poly-L-glutamic acid complex-containing aqueous suspension, a 0.1-fold amount of 0.6% by weight hydrogen peroxide was added, to obtain anti-IgE monoclonal antibody-poly-L-glutamic acid complex-containing aqueous suspension/0.1% hydrogen peroxide. This liquid was kept at 37° C. for two hours. Besides, a 0.1-fold amount of water was added in place of the 0.6% by weight hydrogen peroxide, to obtain a 50.0 mg/mL anti-IgE monoclonal antibody-poly-L-glutamic acid complex-containing aqueous suspension. These liquids were kept at 37° C. for two hours, and then sodium chloride was added thereto so as to attain a concentration of 150 mM. Further, the liquids were subjected to fragmentation by trypsin, and the fragmentation products were subjected to analysis of primary structure by a peptide mapping method. Besides, a liquid obtained by preparing anti-IgE monoclonal antibody in a 10 mM MOPS buffer (pH 5.5) so as to attain a concentration of 60.0 mg/mL was made to be a control liquid (Comparative Example 101), and a 0.1-fold amount of 0.6% by weight hydrogen peroxide was added thereto, to obtain 50.0 mg/mL anti-IgE monoclonal antibody/0.1% hydrogen peroxide. Further, a 0.1-fold amount of water was added in place of the 0.6% by weight hydrogen peroxide, to obtain 50.0 mg/mL anti-IgE monoclonal antibody. These control liquids were kept at 37° C. for two hours, followed by adding sodium chloride thereto so as to attain a concentration of 150 mM. Further, the liquids were subjected to fragmentation by trypsin, and the fragmentation products were subjected to analysis of primary structure by a peptide mapping method. From the results of these operations, rate of change in primary structure in the presence of hydrogen peroxide based on primary structure in the absence of hydrogen peroxide was calculated from rate of decrease in peak area, for the anti-IgE monoclonal antibody-poly-L-glutamic acid complex-containing aqueous suspension and the control liquids. The rate of change in primary structure in the anti-IgE monoclonal antibody-poly-L-glutamic acid complex-containing aqueous suspension (Example 101) was significantly suppressed, as compared with the rate of change in primary structure in the control liquid (Comparative Example 101). Thus, a stabilizing effect against oxidation stress was depicted as to the protein-polyamino acid complex-containing aqueous suspension. The results, with the rate of change in primary structure in the control liquid being taken as 1, were set forth in Table 33.

TABLE 33

| | | | Protein-polyamino acid |
|---|---|---|---|
| | | Control liquid | complex-containing |
| | | (Comparative | aqueous suspension |
| | | Example 101) | (Example 101) |
| Test | Rate of change | 100 | 86.6 |
| Example | in primary | | |
| 31 | structure (%) | | |

### 10. Examples 102 to 106 which Depict Stabilizing Effect Against Thermal Stress

### <L-Asparaginase>

### Example 102

In a 10 mM MOPS buffer (pH 7.0), L-asparaginase was prepared so as to attain a concentration of 1 mg/mL, and poly-L-lysine (MW: 4 kDa to 15 kDa) was added thereto in an amount of 0.05 part by mass based on 1 part by mass of L-asparaginase. The thus prepared liquid was centrifuged, and 90% of the total volume of the centrifuged liquid was removed as supernatant, to obtain an L-asparaginase-poly-L-lysine complex-containing aqueous suspension.

### Test Example 32

In regard of Example 102 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 10.0 mg/mL. The rest of the L-asparaginase-poly-L-lysine complex-containing aqueous suspension was kept at 60° C. or a cold temperature for five minutes, 15 minutes, and 30 minutes, then sodium chloride was added so as to attain a concentration of 150 mM, and the activity of L-asparaginase was measured. Besides, a liquid obtained by preparing L-asparaginase in a 10 mM MOPS buffer (pH 7.0) so as to attain a concentration of 10.0 mg/mL was made to be a control liquid (Comparative Example 102), which was kept at 60° C. or a cold temperature for five minutes, 15 minutes, and 30 minutes, then sodium chloride was added so as to attain a concentration of 150 mM, and the activity of L-asparaginase was measured. From the results of these measurements, the retention rate of L-asparaginase activity when heated based on the L-asparaginase activity when not heated was determined, for the L-asparaginase-poly-L-lysine complex-containing aqueous suspension and the control liquid. In the control liquid, the activity was lowered conspicuously. In the L-asparaginase-poly-L-lysine complex-containing aqueous suspension, on the other hand, the lowering in the activity was significantly suppressed. Thus, a stabilizing effect against thermal stress was depicted as to the protein-polyamino acid complex-containing aqueous suspension. The results were set forth in Table 34.

TABLE 34

| | | Retention rate of activity when heated | |
|---|---|---|---|
| | | based on activity when not heated (%) | |
| | | | Protein-polyamino acid |
| | Time of heating | Control liquid | complex-containing |
| | at 60° C. | (Comparative | aqueous suspension |
| | (min) | Example 102) | (Example 102) |
| Test | 5 | 51.3 | 86.2 |
| Example | 15 | 19.9 | 82.5 |
| 32 | 30 | 6.5 | 84.7 |

### Example 103

In a 10 mM MOPS buffer (pH 7.0), L-asparaginase was prepared so as to attain a concentration of 4.3 mg/mL, to which there was added poly-L-lysine (MW: 4 kDa to 15 kDa) in an amount of 0.05 part by mass, or poly-L-lysine (MW: 15 kDa to 30 kDa) in an amount of 0.05 part by mass, or poly-L-lysine (MW: not less than 30 kDa) in an amount of 0.03 part by mass, or poly-L-arginine (MW: 5 kDa to 15 kDa) in an amount of 0.05 part by mass, based on 1 part by mass of L-asparaginase. The thus prepared liquids were centrifuged, and 90% of the total volume of the centrifuged liquid was removed as supernatant, to obtain an L-asparaginase-poly-L-lysine complex-containing aqueous suspension or an L-asparaginase-poly-L-arginine complex-containing aqueous suspension.

### Test Example 33

In regard of Example 103 above, part of the obtained L-asparaginase-poly-L-lysine complex-containing aqueous suspension or L-asparaginase-poly-L-arginine complex-containing aqueous suspension was sampled, sodium chloride was added thereto so as to attain a concentration of 150 mM, and absorbance at a wavelength of 280 nm was measured as measurement of protein concentration, which was 43.0 mg/mL. The rest of the L-asparaginase-poly-L-lysine complex-containing aqueous suspension or L-asparaginase-poly-L-arginine complex-containing aqueous suspension (Example 103) was put to differential scanning calorimetry. Besides, a liquid obtained by preparing L-asparaginase in a 10 mM MOPS buffer (pH 7.0) so as to attain a concentration of 43.0 mg/mL was made to be a control liquid (Comparative Example 103), which was put to differential scanning calorimetry. For each aqueous suspension or control liquid, denaturing temperature was determined. As a result, it was made clear that in the L-asparaginase-poly-L-lysine complex-containing aqueous suspension or L-asparaginase-poly-L-arginine complex-containing aqueous suspension, denaturation of protein occurs at a high temperature, as compared to the control liquid. Thus, a stabilizing effect against thermal stress was depicted as to the protein-polyamino acid complex-containing aqueous suspension. The results were set forth in Table 35.

TABLE 35

| | | Denaturing | Difference from |
|---|---|---|---|
| | | temperature | control liquid |
| | Polyamino acid used | (° C.) | (° C.) |
| Example | — | 67.13 | — |
| 103 | Poly-L-lysine (4 to 15 kDa) | 68.94 | 1.81 |
| | Poly-L-lysine (15 to 30 | 69.17 | 2.04 |
| | kDa) | | |
| | Poly-L-lysine (30 kDa or | 69.31 | 2.18 |
| | above) | | |
| | Poly-L-arginine (5 to 15 | 69.54 to | +2.41 to +4.74 |
| | kDa) | 71.87 | |

### <Anti-IgE Monoclonal Antibody>

### Example 104

In a 10 mM Tris-HCl buffer (pH 8.7), anti-IgE monoclonal antibody was prepared so as to attain a concentration of 1.25 mg/mL, and poly-L-lysine (MW: 4 kDa to 15 kDa) was added thereto in an amount of 0.1 part by mass based on 1 part by mass of anti-IgE monoclonal antibody. The thus prepared liquid was centrifuged, and 90% of the total volume of the centrifuged liquid was removed as supernatant, to obtain an anti-IgE monoclonal antibody-poly-L-lysine complex-containing aqueous suspension.

### Test Example 34

In regard of Example 104 above, part of the obtained anti-IgE monoclonal antibody-poly-L-lysine complex-containing aqueous suspension was sampled, sodium chloride was added thereto so as to attain a concentration of 150 mM, and absorbance at a wavelength of 280 nm was measured as measurement of protein concentration, which was 10.6 mg/mL. The rest of the anti-IgE monoclonal antibody-poly-L-lysine complex-containing aqueous suspension was kept at 60° C. or a cold temperature for 15 hours, sodium chloride was added so as to attain a concentration of 150 mM, and the activity of anti-IgE monoclonal antibody was measured. Besides, a liquid obtained by preparing anti-IgE monoclonal antibody in a 10 mM MOPS buffer (pH 7.0) so as to attain a concentration of 10.6 mg/mL was made to be a control liquid. The control liquid was kept at 60° C. or a cold temperature for 15 hours, sodium chloride was added thereto so as to attain a concentration of 150 mM, and the activity of anti-IgE monoclonal antibody was measured. From the results of these measurements, the retention rate of the activity of anti-IgE monoclonal antibody when heated based on the activity of anti-IgE monoclonal antibody when not heated was determined, for the anti-IgE monoclonal antibody-poly-L-lysine complex-containing aqueous suspension and the control liquid. In the control liquid, the activity was lowered. In the anti-IgE monoclonal antibody-poly-L-lysine complex-containing aqueous suspension, on the other hand, the lowering in the activity was suppressed. Thus, a stabilizing effect against thermal stress was depicted as to the protein-polyamino acid complex-containing aqueous suspension. The results were set forth in Table 36.

TABLE 36

| | | | Protein-polyamino acid |
|---|---|---|---|
| | | Control liquid | complex-containing |
| | | (Comparative | aqueous suspension |
| | | Example 104) | (Example 104) |
| Test | Retention rate of | 72.9 | 86 |
| Example | activity when | | |
| 34 | heated based on | | |
| | activity when not | | |
| | heated (%) | | |

### Example 105

In a 10 mM citrate buffer (pH 5.5), anti-IgE monoclonal antibody was prepared so as to attain a concentration of 5 mg/mL, and poly-L-glutamic acid (MW: 3 kDa to 15 kDa) was added thereto in an amount of 0.05 part by mass based on 1 part by mass of anti-IgE monoclonal antibody. The thus prepared liquid was centrifuged, and 90% of the total volume of the centrifuged liquid was removed as supernatant, to obtain an anti-IgE monoclonal antibody-poly-L-glutamic acid complex-containing aqueous suspension.

### Test Example 35

In regard of Example 105 above, part of the obtained anti-IgE monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 50 mg/mL. To the rest of the anti-IgE monoclonal antibody-poly-L-glutamic acid complex-containing aqueous suspension, a ninefold amount of a 10 mM citrate buffer (pH 5.5) was added to attain a concentration of 5 mg/mL, then the resulting liquid was kept at 60° C. or a cold temperature for 15 hours, or at 50° C. or a cold temperature for 60 hours, after which sodium chloride was added so as to attain a concentration of 150 mM, and the activity of anti-IgE monoclonal antibody was measured. Besides, a liquid obtained by preparing anti-IgE monoclonal antibody in a 10 mM citrate buffer (pH 5.5) so as to attain a concentration of 5 mg/mL was made to be a control liquid, which was kept at 60° C. or a cold temperature for 15 hours, or at 50° C. or a cold temperature for 60 hours, thereafter sodium chloride was added so as to attain a concentration of 150 mM, and the activity of anti-IgE monoclonal antibody was measured. From the results of these measurements, the retention rate of the activity of anti-IgE monoclonal antibody when heated based on the activity of anti-IgE monoclonal antibody when not heated was determined, for the anti-IgE monoclonal antibody-poly-L-glutamic acid complex-containing aqueous suspension and the control liquid. In the control liquid, the activity was lowered. In the anti-IgE monoclonal antibody-poly-L-glutamic acid complex-containing aqueous suspension, on the other hand, the lowering of the activity was restrained. Thus, a stabilizing effect against thermal stress was depicted as to the protein-polyamino acid complex-containing aqueous suspension. The results were set forth in Table 37.

TABLE 37

| | | Retention rate of activity when heated | |
|---|---|---|---|
| | | based on activity when not heated (%) | |
| | | | Protein-polyamino acid |
| | | Control liquid | complex-containing |
| | Heating | (Comparative | aqueous suspension |
| | conditions | Example 105) | (Example 105) |
| Test | 60° C. × 16 hr | 51.8 | 84.3 |
| Example | 50° C. × 60 hr | 43.2 | 86.0 |
| 35 | | | |

### Example 106

In a 10 mM citrate buffer (pH 5.4), anti-IgE monoclonal antibody was prepared so as to attain a concentration of 5.0 mg/mL, and poly-L-glutamic acid (MW: 3 kDa to 15 kDa) was added thereto in an amount of 0.075 part by mass based on 1 part by mass of anti-IgE monoclonal antibody. The thus prepared liquid was centrifuged, and 90% of the total volume of the centrifuged liquid was removed as supernatant, to obtain an anti-IgE monoclonal antibody-poly-L-glutamic acid complex-containing aqueous suspension.

### Test Example 36

In regard of Example 106 above, part of the obtained anti-IgE monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 50.0 mg/mL. The rest of the anti-IgE monoclonal antibody-poly-L-glutamic acid complex-containing aqueous suspension was put to differential scanning calorimetry. Besides, a liquid obtained by preparing anti-IgE monoclonal antibody in a 10 mM citrate buffer (pH 5.4) so as to attain a concentration of 50.0 mg/mL was made to be a control liquid, which was put to differential scanning calorimetry. For each of the aqueous suspension and the control liquid, denaturing temperature was determined. It was made clear that in the anti-IgE monoclonal antibody-poly-L-glutamic acid complex-containing aqueous suspension, denaturation of protein occurs at a high temperature, as compared to the control liquid. Thus, a stabilizing effect against thermal stress was depicted as to the protein-polyamino acid complex-containing aqueous suspension. The results were set forth in Table 38.

TABLE 38

| | | Denaturing | Denaturing |
|---|---|---|---|
| | | temperature 1 | temperature 2 |
| | | (° C.) | (° C.) |
| Test | Control liquid | 71.7 | 83.41 |
| Example | (Comparative | | |
| 36 | Example 106) | | |
| | Protein-polyamino acid | 79.44 | 90 |
| | complex-containing | | |
| | aqueous suspension | | |
| | (Example 106) | | |

### 11. Examples 107 to 110 which Depict Aggregation Inhibitory Effect

### <Anti-EGFR Monoclonal Antibody>

### Example 107

In a 10 mM MOPS buffer (pH 5.0), anti-EGFR monoclonal antibody was prepared so as to attain a concentration of 5.3 mg/mL, and poly-L-glutamic acid (MW: 3 kDa to 15 kDa) was added thereto in an amount of 0.05 part by mass based on 1 part by mass of anti-EGFR monoclonal antibody. The thus prepared liquid was centrifuged, and 94% of the total volume of the centrifuged liquid was removed as supernatant, to obtain an anti-EGFR monoclonal antibody-poly-L-glutamic acid complex-containing aqueous suspension.

### Test Example 37

In regard of Example 107 above, part of the obtained anti-EGFR monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 89.0 mg/mL. The rest of the anti-EGFR monoclonal antibody-poly-L-glutamic acid complex-containing aqueous suspension was filled into a polypropylene-made disposable syringe, which was placed on a shaker (Bioshaker V⋅BR-36), and shaking at 500 rpm was conducted at room temperature for 90 hours. Sodium chloride was added to the anti-EGFR monoclonal antibody-poly-L-glutamic acid complex-containing aqueous suspension before the shaking and to that after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration (Example 107). Besides, a liquid obtained by preparing anti-EGFR monoclonal antibody in a 10 mM MOPS buffer (pH 5.0) so as to attain a concentration of 89.0 mg/mL was made to be a control liquid (Comparative Example 107), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration. From the results of these measurements, for the anti-EGFR monoclonal antibody-poly-L-glutamic acid complex-containing aqueous suspension and the control liquid, the amount of insoluble aggregates of the protein was calculated by subtracting the mass of the protein in the supernatant from the total mass of the protein, and the rate of conversion from protein before shaking into insoluble protein aggregates after shaking was determined. In the anti-EGFR monoclonal antibody-poly-L-glutamic acid complex-containing aqueous suspension, the rate of conversion from protein into insoluble protein aggregates was suppressed, as compared to the control liquid. Thus, an aggregation inhibitory effect of the protein-polyamino acid complex-containing aqueous suspension was depicted. The results were set forth in Table 39.

TABLE 39

| | | | Protein-polyamino acid |
|---|---|---|---|
| | | Control liquid | complex-containing |
| | | (Comparative | aqueous suspension |
| | | Example 107) | (Example 107) |
| Test | Rate of | 10.9 | 1.6 |
| Example | conversion from | | |
| 37 | protein before | | |
| | shaking into | | |
| | insoluble protein | | |
| | aggregates after | | |
| | shaking (%) | | |

### Example 108

In a 10 mM MOPS buffer (pH 5.0), anti-EGFR monoclonal antibody was prepared so as to attain a concentration of 5.3 mg/mL, and poly-L-glutamic acid (MW: 3 kDa to 15 kDa) was added thereto in an amount of 0.05 part by mass based on 1 part by mass of anti-EGFR monoclonal antibody. The thus prepared liquid was centrifuged, and 95% of the total volume of the centrifuged liquid was removed as supernatant, to obtain anti-EGFR monoclonal antibody-poly-L-glutamic acid complex-containing aqueous suspension.

### Test Example 38

In regard of Example 108 above, part of the obtained anti-EGFR monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 114.9 mg/mL. The rest of the anti-EGFR monoclonal antibody-poly-L-glutamic acid complex-containing aqueous suspension was filled into a polypropylene-made disposable syringe, which was placed on a shaker (Bioshaker V⋅BR-36), and shaking at 500 rpm was conducted at room temperature for 90 hours. Sodium chloride was added to the anti-EGFR monoclonal antibody-poly-L-glutamic acid complex-containing aqueous suspension before the shaking and to that after the shaking so as to attain a concentration of 150 mM, the resulting liquids were centrifuged at 10,000 g for 15 minutes, and the supernatants were put to measurement of absorbance at a wavelength of 280 nm as measurement of protein concentration (Example 108). Besides, a liquid obtained by preparing anti-EGFR monoclonal antibody in a 10 mM MOPS buffer (pH 5.0) so as to attain a concentration of 114.9 mg/mL was made to be a control liquid (Comparative Example 108), which was filled into a syringe, and subjected to shaking simultaneously with the aqueous suspension. Sodium chloride was added to the control liquid before the shaking and to that after the shaking so as to attain a concentration of 150 mM, the resulting liquids were centrifuged at 10,000 g for 15 minutes, and the supernatants were subjected to measurement of absorbance at a wavelength of 280 nm as measurement of protein concentration. From the results of these measurements, for the anti-EGFR monoclonal antibody-poly-L-glutamic acid complex-containing aqueous suspension and the control liquid, the amount of insoluble aggregates of protein was calculated by subtracting the mass of the protein in the supernatant from the total mass of the protein, and the rate of conversion from the protein before the shaking into the insoluble protein aggregates after the shaking was determined. As a result, aggregation of protein was not recognized in the anti-EGFR monoclonal antibody-poly-L-glutamic acid complex-containing aqueous suspension. Thus, an aggregation inhibitory effect was depicted as to the protein-polyamino acid complex-containing aqueous suspension. The results were set forth in Table 40.

TABLE 40

| | | | Protein-polyamino acid |
|---|---|---|---|
| | | Control liquid | complex-containing |
| | | (Comparative | aqueous suspension |
| | | Example 108) | (Example 108) |
| Test | Rate of | 12.8 | 0 |
| Example | conversion from | | |
| 38 | protein before | | |
| | shaking into | | |
| | insoluble protein | | |
| | aggregates after | | |
| | shaking (%) | | |

### <L-Asparaginase>

### Example 109

In a 10 mM MOPS buffer (pH 7.0), L-asparaginase was prepared so as to attain a concentration of 1 mg/mL, and poly-L-lysine (MW: 4 kDa to 15 kDa) was added thereto in an amount of 0.05 part by mass based on 1 part by mass of L-asparaginase. The thus prepared liquid was centrifuged, and 90% of the total volume of the centrifuged liquid was removed as supernatant, to obtain an L-asparaginase-poly-L-lysine complex-containing aqueous suspension.

### Test Example 39

In regard of Example 109 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 10.0 mg/mL. After the rest of the L-asparaginase-poly-L-lysine complex-containing aqueous suspension (Example 109) was kept at 60° C. or room temperature for five minutes, sodium chloride was added thereto so as to attain a concentration of 150 mM, and the number of aggregates in the resulting liquid was measured by a micro-flow imaging (MFI) method. Besides, a liquid obtained by preparing L-asparaginase in a 10 mM MOPS buffer (pH 7.0) so as to attain a concentration of 10.0 mg/mL was made to be a control liquid (Comparative Example 109), which was kept at 60° C. or room temperature for five minutes, thereafter sodium chloride was added thereto so as to attain a concentration of 150 mM, and the number of aggregates in the resulting liquid was measured by a micro-flow imaging method. From the results of these measurements, for the L-asparaginase-poly-L-lysine complex-containing aqueous suspension and the control liquid, the rate of increase in the number of aggregates was determined by dividing the number of aggregates when heated by the number of aggregates when not heated. The aggregate increase rate in the L-asparaginase-poly-L-lysine complex-containing aqueous suspension was found significantly lower than the aggregate increase rate in the control liquid. Thus, an aggregation inhibitory effect was depicted as to the protein-polyamino acid complex-containing aqueous suspension. The results were set forth in Table 41.

### Test Example 40

In regard of Example 109 above, part of the obtained L-asparaginase-poly-L-lysine complex-containing aqueous suspension was sampled, sodium chloride was added thereto so as to attain a concentration of 150 mM, and absorbance at a wavelength of 280 nm was measured as measurement of protein concentration, which was 10.0 mg/mL. After the rest of the L-asparaginase-poly-L-lysine complex-containing aqueous suspension was kept at 60° C. or room temperature for five minutes, sodium chloride was added thereto so as to attain a concentration of 150 mM, and absorbance at a wavelength of 600 nm was measured as measurement of turbidity. Besides, a liquid obtained by preparing L-asparaginase in a 10 mM MOPS buffer (pH 7.0) so as to attain a concentration of 10.0 mg/mL was made to be a control liquid, which was kept at 60° C. or room temperature for five minutes, thereafter sodium chloride was added thereto so as to attain a concentration of 150 mM, and absorbance at a wavelength of 600 nm was measured as measurement of turbidity. From the results of these measurements, for the L-asparaginase-poly-L-lysine complex-containing aqueous suspension and the control liquid, the rate of increase in turbidity was determined by dividing the turbidity when heated by the turbidity when not heated. The rate of increase in turbidity of the L-asparaginase-poly-L-lysine complex-containing aqueous suspension was significantly lower than the rate of increase in turbidity of the control liquid. Thus, an aggregation inhibitory effect was depicted as to the protein-polyamino acid complex-containing aqueous suspension. The results were set forth in Table 41.

TABLE 41

| | | | Protein-polyamino acid |
|---|---|---|---|
| | | Control liquid | complex-containing |
| | | (Comparative | aqueous suspension |
| | | Example 109) | (Example 109) |
| Test | Rate of increase | 3156.3 | 9.7 |
| Example | in number of | | |
| 39 | aggregates after | | |
| | heating based on | | |
| | number of | | |
| | aggregate before | | |
| | heating (times) | | |
| Test | Rate of increase | 206.9 | 7.4 |
| Example | in turbidity | | |
| 40 | after heating | | |
| | based on | | |
| | turbidity before | | |
| | heating (times) | | |

### <Anti-TNFα Monoclonal Antibody>

### Test Example 41

In regard of Example 95 above, part of the obtained anti-TNFα monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 50.0 mg/mL. To the rest of the anti-TNFα monoclonal antibody-poly-L-glutamic acid complex-containing aqueous suspension, a ninefold amount of a 10 mM MOPS buffer (pH 6.5) was added to attain a protein concentration of 5.0 mg/mL, the resulting aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-TNFα monoclonal antibody-poly-L-glutamic acid complex-containing aqueous suspension before the shaking and to that after the shaking so as to attain a concentration of 150 mM, and the number of aggregates was measured by a micro-flow imaging (MFI) method. Besides, a liquid obtained by preparing anti-TNFα monoclonal antibody in a 10 mM MOPS buffer (pH 6.5) so as to attain a concentration of 5.0 mg/mL was made to be a control liquid (Comparative Example 95), which was filled into a syringe, and subjected to shaking simultaneously with the aqueous suspension. Sodium chloride was added to the control liquid before the shaking and to that after the shaking so as to attain a concentration of 150 mM, and the number of aggregates was measured by a micro-flow imaging method. From the results of these measurements, for the anti-TNFα monoclonal antibody-poly-L-glutamic acid complex-containing aqueous suspension and the control liquid, the rate of increase in the number of aggregates was determined by dividing the number of aggregates after the shaking by the number of aggregates before the shaking. The rate of increase in the number of aggregates in the anti-TNFα monoclonal antibody-poly-L-glutamic acid complex-containing aqueous suspension was significantly lower than the rate of increase in the number of aggregates in the control liquid. Thus, an aggregation inhibitory effect was depicted as to the protein-polyamino acid complex-containing aqueous suspension. The results were set forth in Table 42.

TABLE 42

| | | | Protein-polyamino acid |
|---|---|---|---|
| | | Control liquid | complex-containing |
| | | (Comparative | aqueous suspension |
| | | Example 95) | (Example 95) |
| Test | Rate of increase | 2121.5 | 2.2 |
| Example | in number of | | |
| 41 | aggregates after | | |
| | shaking based on | | |
| | number of | | |
| | aggregates before | | |
| | shaking (times) | | |

### <Anti-IgE Monoclonal Antibody>

### Example 110

In a 10 mM citrate buffer (pH 5.4), anti-IgE monoclonal antibody was prepared so as to attain a concentration of 5.0 mg/mL, and poly-L-glutamic acid (MW: 3 kDa to 15 kDa) was added thereto in an amount of 0.1 part by mass based on 1 part by mass of anti-IgE monoclonal antibody. The thus prepared liquid was centrifuged, and 90% of the total volume of the centrifuged liquid was removes as supernatant, to obtain an anti-IgE monoclonal antibody-poly-L-glutamic acid complex-containing aqueous suspension.

### Test Example 42

In regard of Example 110 above, part of the obtained anti-IgE monoclonal antibody-poly-L-glutamic acid complex-containing aqueous suspension was sampled, sodium chloride was added thereto so as to attain a concentration of 150 mM, and absorbance at a wavelength of 280 nm was measured as measurement of protein concentration, which was 50.0 mg/mL. To the rest of the anti-IgE monoclonal antibody-poly-L-glutamic acid complex-containing aqueous suspension, a ninefold amount of a 10 mM citrate buffer (pH 5.4) was added to attain a protein concentration of 5.0 mg/mL, the resulting aqueous suspension was filled into a polypropylene-made disposable syringe, which was placed on a shaker (Bioshaker V⋅BR-36), and shaking at 500 rpm was conducted at room temperature for 60 hours. Sodium chloride was added to the anti-IgE monoclonal antibody-poly-L-glutamic acid complex-containing aqueous suspension before the shaking and to that after the shaking so as to attain a concentration of 150 mM, and the resulting aqueous suspensions were analyzed by size exclusion chromatography (SEC) (Example 110). Besides, a liquid obtained by preparing anti-IgE monoclonal antibody in a 10 mM citrate buffer (pH 5.4) so as to attain a concentration of 5.0 mg/mL was made to be a control liquid (Comparative Example 110), which was filled into a syringe, and was subjected to shaking simultaneously with the aqueous suspension. Sodium chloride was added to the control liquid before the shaking and to that after the shaking so as to attain a concentration of 150 mM, and the resulting liquids were analyzed by size exclusion chromatography. From the results of these measurements, for the anti-IgE monoclonal antibody-poly-L-glutamic acid complex-containing aqueous suspension and the control liquid, peak area of soluble aggregates was determined. In the control liquid, the soluble aggregate peak area depicted a conspicuous increase. In the anti-IgE monoclonal antibody-poly-L-glutamic acid complex-containing aqueous suspension, on the other hand, the increase of soluble aggregate peak area was significantly suppressed. Thus, an aggregation inhibitory effect was depicted as to the protein-polyamino acid complex-containing aqueous suspension. The results were set forth in Table 43.

TABLE 43

| | | | Protein-polyamino acid |
|---|---|---|---|
| | | Control liquid | complex-containing |
| | | (Comparative | aqueous suspension |
| | | Example 110) | (Example 110) |
| Test | Rate of increase | 178.1 | 8.5 |
| Example | of soluble | | |
| 42 | aggregate peak | | |
| | area after shaking | | |
| | based on soluble | | |
| | aggregate peak | | |
| | area before | | |
| | shaking (%) | | |

### 12. Comparative Experiment in which Synthetic Polymer is Used

### Control Examples 111 and 112

(1) Confirmation of that Condensation and about 100% Recovery of L-Asparaginase can be Made Using Polyethyleneimine

In a 10 mM MOPS buffer (pH 7.0), L-asparaginase was prepared so as to attain a concentration of 1.0 mg/mL, and polyallylamine (MW: 5 kDa) or polyethyleneimine (MW: 1.8 kDa) was added thereto in an amount of 0.05 part by mass based on 1 part by mass of L-asparaginase. Each of these prepared liquids was centrifuged, and 90% of the total volume of the centrifuged liquid was removed as supernatant, to obtain aqueous suspensions containing a complex of L-asparaginase and each polymer.

### Test Example 43

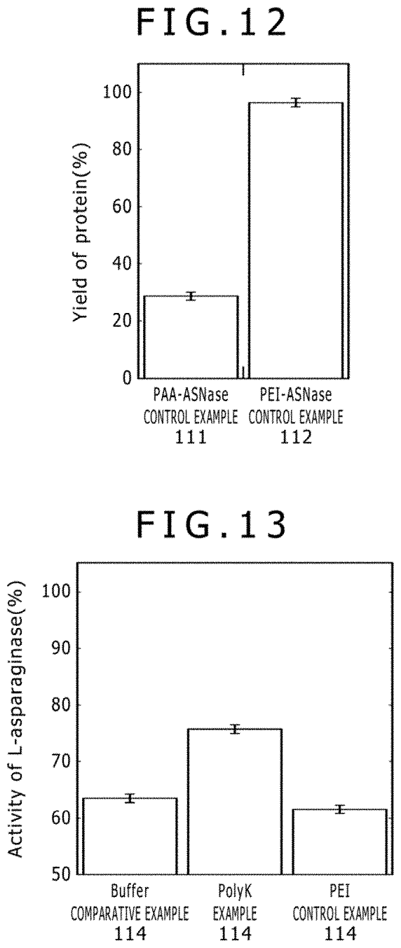

In regard of Control Examples 111 and 112 above, parts of the obtained L-asparaginase-polyallylamine complex-containing aqueous suspension (Control Example 111) and the obtained L-asparaginase-polyethyleneiminde complex-containing aqueous suspension (Control Example 112) were sampled, sodium chloride was added to each of the samples so as to attain a concentration of 150 mM, and absorbance at a wavelength of 280 nm was measured as measurement of protein concentration. While the recovery factor of L-asparaginase from the L-asparaginase-polyethyleneimine complex-containing aqueous suspension was close to 100%, the recovery factor of L-asparaginase from the L-asparaginase-polyallylamine complex-containing aqueous suspension was significantly lower. This depicted that polyethyleneimine forms a complex with L-asparaginase, in the same manner as polylysine (see Examples 1 to 11), and enables concentration and recovery of L-asparaginase. The results are depicted in FIG. 12.

### Control Example 113

(2) Confirmation of that Anti-TNFα Monoclonal Antibody can be Concentrated Using Polyacrylic Acid

In a 10 mM MOPS buffer (pH 7.0), anti-TNFα monoclonal antibody was prepared so as to attain a concentration of 0.5 mg/mL, and polyacrylic acid (MW: 5 kDa) was added thereto in an amount of 0.04 part by mass based on 1 part by mass of anti-TNFα monoclonal antibody (Control Example 113). Each of the prepared liquids was centrifuged, and 90% of the total volume of the centrifuged liquid was removed as supernatant, to obtain an anti-TNFα monoclonal antibody-polyacrylic acid complex-containing aqueous suspension. Besides, in Comparative Example 113, the same operations as above were conducted, except for not adding polyacrylic acid.

### Test Example 44

Sodium chloride was added to this liquid so as to attain a concentration of 150 mM, the resulting liquid was centrifuged, and the resulting supernatant was put to measurement of absorbance at a wavelength of 280 nm as measurement of protein concentration. The results are set forth in Table 44. The protein concentration ratio in Control Example 113 based on the protein concentration in Comparative Example 113 was determined. The protein concentration rose to about 9.9 times. Thus it was depicted that anti-TNFα monoclonal antibody can be concentrated using polyacrylic acid. The results are set forth in Table 44.

TABLE 44

| | | Comparative | Control |
|---|---|---|---|
| | | Example 113 | Example 113 |
| Test Example using | Polyacrylic acid/ | 0 | 0.04 |
| anti-TNFα | protein mass ratio | | |
| monoclonal | Protein concentration | 1 | 9.9 |
| antibody | ratio | | |

### Control Example 114

(3) Comparison of Stabilizing Effect Against Shaking Stress by Formation of Complex of Polylysine or Polyethyleneimine with L-Asparaginase

In a 10 mM MOPS buffer (pH 7.0), L-asparaginase was prepared so as to attain a concentration of 1.0 mg/mL, and poly-L-lysine (MW: 4 kDa to 15 kDa) (Example 114) of polyethyleneimine (MW: 1.8 kDa) (Control Example 114) was added thereto in an amount of 0.05 part by mass based on 1 part by mass of L-asparaginase. Each of these prepared liquids wad centrifuged, and 90% of the total volume of the centrifuged liquid was removed as supernatant, to obtain aqueous suspensions containing a complex of L-asparaginase with each polymer.

### Test Example 45

To each of the liquids prepared in Example 114 and Control Example 114, a ninefold amount of a 10 mM MOPS buffer (pH 7.0) was added to obtain a protein concentration of 10.0 mg/mL, and each of the resulting liquids was filled into a polypropylene-made tube, which was placed on a shaker (Bioshaker V⋅BR-36), and shaking at 500 rpm was conducted at room temperature for 60 hours. Sodium chloride was added to the aqueous suspensions containing a complex of L-asparaginase with each polymer before the shaking and to those after the shaking so as to attain a concentration of 150 mM, the resulting aqueous suspensions were centrifuged at 10,000 g for 15 minutes, and the supernatants were put to measurement of absorbance at a wavelength of 280 nm as measurement of protein concentration. Besides, a liquid obtained by preparing L-asparaginase in a 10 mM MOPS buffer (pH 7.0) so as to attain a concentration of 10.0 mg/mL was made to be a control liquid (Comparative Example 114), which was filled into a polypropylene-made tube, and subjected to shaking simultaneously with the aqueous suspensions. Sodium chloride was added to the control liquid before the shaking and to that after the shaking so as to attain a concentration of 150 mM, the resulting liquids were centrifuged at 10,000 g for 15 minutes, and the supernatant was put to measurement of absorbance at a wavelength of 280 nm as measurement of protein concentration. For the aqueous suspensions containing the complex of L-asparaginase with each polymer and the control liquid, the retention rate of protein content after the shaking based on the protein content before the shaking was determined. The results are depicted in FIG. 13. The retained activity in the L-asparaginase-polyethyleneimine complex-containing aqueous suspension (Control Example 114) was significantly lower than the retained activity in the L-asparaginase-polylysine complex-containing aqueous suspension (Example 114), and was comparable to the retained activity in Comparative Example 114. Thus, it was depicted that the L-asparaginase-polyethyleneimine complex-containing aqueous suspension does not have shaking stress resistance.

### (4) Confirmation of Cytotoxicity of Polyglutamic Acid and Polyacrylic Acid

Polyglutamic acid (MW: 3 kDa to 15 kDa), polyacrylic acid (MW: 5 kDa), and polyacrylic acid (MW: 25 kDa) were individually dissolved in a culture medium, and, for each of the polymers, various polymer solutions were thereby prepared so as to attain concentrations of 0% to 1%. Each of the 0% to 1% solutions of each of the polymers was added onto CHO cells seeded on a 96-well plate, and incubation was conducted in a CO2 incubator for 18 hours. After 18 hours, cell growth rate in the case of incubation under each polymer solution was compared with cell growth rate in the case of incubation in a culture medium not containing any polymer (0% polymer solution). As a result, it was depicted that in the case of the polyacrylic acid solution (MW: both 5 kDa and 25 kDa), inhibition of CHO cell growth starts from a concentration of about 0.05% to 0.1%, and the concentration for 50% inhibition of cell growth is around 0.17%. In the case of polyglutamic acid solution, on the other hand, CHO cell growth was little inhibited even with a 1% solution. The results are set forth in Table 45.

TABLE 45

| | 50% cell growth inhibitory |
|---|---|
| Object polymer | concentration |
| Poly-L-glutamic acid | 1.00% or above |
| Polyacrylic acid (5 kDa) | 0.17% |
| Polyacrylic acid (25 kDa) | 0.17% |

### 13. Confirmation of Salt Concentration Required for Re-Dissolution of Complex

### <Confirmation of Salt Concentration Required for Dissociation of Anti-TNFα Monoclonal Antibody-Polyglutamic Acid Complex>

In a 10 mM MOPS buffer (pH 7.0), anti-TNFα antibody was prepared so as to attain a concentration of 0.5 mg/mL, and poly-L-glutamic acid (MW: 3 kDa to 15 kDa) was added thereto in an amount of 0.05 part by mass based on 1 part by mass of anti-TNFα antibody. Each of these prepared liquids was centrifuged, and 90% of the total volume of the centrifuged liquid was removed as supernatant, to obtain an anti-TNFα monoclonal antibody-poly-L-glutamic acid complex-containing aqueous suspension. To the thus prepared aqueous suspension, a ninefold amount of each of NaCl-containing 10 mM MOPS buffer (pH 7.0) so prepared as to have a NaCl concentration of 0 mM to 100 mM (0% to 0.6% by weight) was added, the resulting liquids were centrifuged, and the supernatants were put to measurement of absorbance at a wavelength of 280 nm as measurement of protein concentration. The ratio of protein concentration in the supernatant after the NaCl addition and centrifugation based on the initial protein concentration was determined. A rise in the protein concentration was confirmed at a NaCl concentration of not less than 10 mM (0.06% by weight). From this result, it was depicted that a NaCl concentration of not less than 10 mM (0.06% by weight) is required for re-dissolution of the anti-TNFα monoclonal antibody-poly-L-glutamic acid complex-containing aqueous suspension, namely, for dissociation of the anti-TNFα monoclonal antibody from the complex. The results are set forth in Table 46.

TABLE 46

| | NaCl concentration (mM) | | | | | | | | | | |
|---|---|---|---|---|---|---|---|---|---|---|---|
| | 0 | 2 | 4 | 6 | 8 | 10 | 20 | 40 | 60 | 80 | 100 |
| Protein | 0.1 | 0.1 | 0.1 | 0.1 | 0.1 | 0.2 | 1.8 | 8 | 9.1 | 8.1 | 9.2 |
| concentration | | | | | | | | | | | |
| ratio | | | | | | | | | | | |

### <Confirmation of Salt Concentration Required for Dissociation of L-Asparaginase-Polylysine Complex>

In a 10 mM MOPS buffer (pH 7.0), L-asparaginase was prepared so as to attain a concentration of 0.5 mg/mL, and poly-L-lysine (MW: 4 kDa to 15 kDa) was added thereto in an amount of 0.05 part by mass based on 1 part by mass of L-asparaginase. Each of these prepared liquids was centrifuged, and 90% of the total volume of the centrifuged liquid was removed as supernatant, to obtain an L-asparaginase-poly-L-lysine complex-containing aqueous suspension. To the prepared aqueous suspension, a ninefold amount of each of NaCl-containing 10 mM MOPS buffers (pH 7.0) prepared so as to have NaCl concentrations of 0 mM to 100 mM (0% to 0.6% by weight) was added, the resulting aqueous suspensions were centrifuged, and the supernatants were put to measurement of absorbance at a wavelength of 280 nm as measurement of protein concentration. The ratio of protein concentration in the supernatant after the NaCl addition and centrifugation based on the initial protein concentration was determined. As a result, a rise in protein concentration was confirmed at NaCl concentrations of not less than 8 mM (0.048% by weight). From this result, it was depicted that a NaCl concentration of not less than 8 mM (0.048% by weight) is required for re-dissolution of the L-asparaginase-poly-L-lysine complex-containing aqueous suspension, namely, for dissociation of L-asparaginase from the complex. The results are set forth in Table 47.

TABLE 47

| | NaCl concentration (mM) | | | | | | | | | |
|---|---|---|---|---|---|---|---|---|---|---|
| | 0 | 4 | 6 | 8 | 10 | 20 | 40 | 60 | 80 | 100 |
| Protein | 0.2 | 0.2 | 0.2 | 0.5 | 0.9 | 4.3 | 8.6 | 9.8 | 9.5 | 9.8 |
| concentration | | | | | | | | | | |
| ratio | | | | | | | | | | |

### 14. Toxicity Test of Complex-Containing Aqueous Suspension Preparation

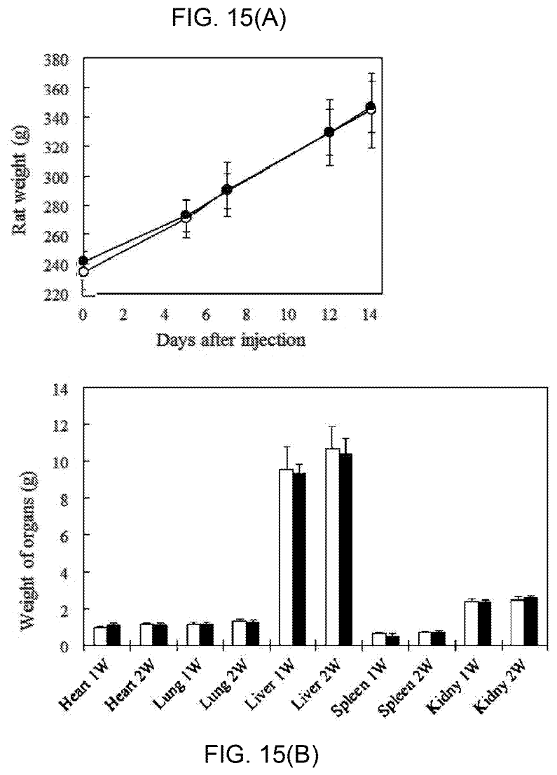

In a 10 mM citrate buffer (pH 5.0), rat IgG was prepared so as to attain a concentration of 1 mg/mL, and poly-L-glutamic acid (MW: 50 kDa to 100 kDa) was added thereto in an amount of 0.12 part by mass based on 1 part by mass of rat IgG. The thus prepared liquid was centrifuged, and 90% of the total volume of the centrifuged liquid was removed as supernatant, to obtain a rat IgG-poly-L-glutamic acid complex-containing aqueous suspension. Besides, as a control liquid, rat IgG was prepared in a 10 mM citrate buffer (pH 5.0) so as to attain a concentration of 10 mg/mL. The aqueous suspension and the control liquid were put to subcutaneous injection into a rat's back in a dose of 50 mg/kg. Thereafter, the body weight and the weights of such organs as the heart, lung, liver, spleen, and kidney of the rat were measured. The results are depicted in FIGS. 15(A) and 15(B). Between the control liquid administration group (white circles and white bars) and the complex-containing aqueous suspension administration group (black circles and black bars), no significant difference was found in the body weight or in the weights of the organs (FIGS. 15(A) and 15(B)). Further, pathology examination of the above-mentioned organs and administered parts and blood examination were carried out. All the organs and administered parts were free of any pathological change. Further, between the control liquid administration group and the complex-containing aqueous suspension administration group, there was no significant difference in any of the blood examination items. From these results, it was confirmed that the disclosed protein-polyamino acid complex-containing aqueous suspension is free of toxicity.

### 15. Confirmation of Transition of Protein Concentration in Plasma Upon Subcutaneous Administration of Protein-Polyamino Acid Complex-Containing Aqueous Suspension Preparation

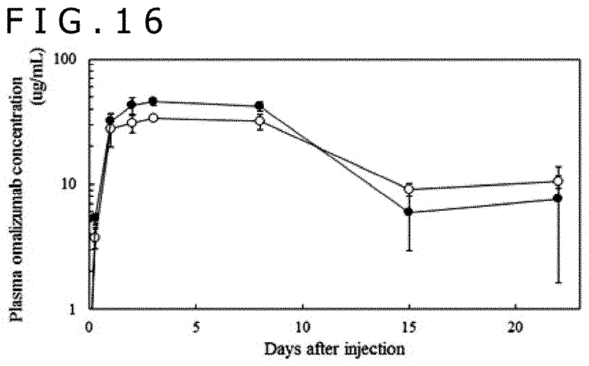

In a 10 mM citrate buffer (pH 5.5), anti-IgE monoclonal antibody was prepared so as to attain a concentration of 1 mg/mL, and poly-L-glutamic acid (MW: 50 kDa to 100 kDa) was added thereto in an amount of 0.05 part by mass based on 1 part by mass of anti-IgE monoclonal antibody. Each of these prepared liquids was centrifuged, and 90% of the total volume of the centrifuged liquid was removed as supernatant, to obtain an anti-IgE monoclonal antibody-poly-L-glutamic acid complex-containing aqueous suspension. Besides, as a control liquid, anti-IgE monoclonal antibody was prepared so as to attain a concentration of 10 mg/mL. The aqueous suspension and the control liquid were put to subcutaneous injection into a rat's back in a dose of 10 mg/kg. Thereafter, the concentration of anti-IgE monoclonal antibody in the rat's blood plasma was determined by an ELISA method. The results are depicted in FIG. 16. The plasma anti-IgE monoclonal antibody concentration-time curve for the control liquid administration group (white circles) and that for the complex-containing aqueous suspension administration group (black circles) coincided with each other. Thus, it was confirmed that the control liquid and the aqueous suspension exhibit the same behavior after subcutaneous injection.

The disclosed embodiments possess significant utility and exhibit one or some of the following characteristics.

It is an aqueous suspension preparation which is stable against vibration during transportation and against thermal stress or oxidation stress during storage, in regard of a wide variety of proteins. The disclosed complex can achieve substantial stabilization and excellent stability during transportation and during storage. The disclosed complex can concentrate a protein to a high concentration easily, without need for special equipment such as one for ultrafiltration. A small lowering in activity due to concentration can be attained. The disclosed aqueous suspension preparation can stabilize a protein easily by only selecting the pH of a buffer and a polyamino acid according to the isoelectric point of the protein, without requiring the addition of an additive or additives which has been conventionally needed. The complex-containing aqueous suspension can be administered as it is as a preparation, without requiring a complicated dissolving operation that is necessary in the case of freeze-dried preparations. Alternatively, the complex-containing aqueous suspension can be administered as an aqueous liquid by re-dissolving it through addition of an inorganic salt represented by sodium chloride. The disclosed aqueous suspension preparation has a characteristic feature of being low in viscosity even when the protein is present at a high concentration, so that the amount of the aqueous suspension preparation left in a container and wasted at the time of use thereof can be reduced. In addition, when administered by a syringe, the aqueous suspension preparation can be administered with a weak force, as compared with an aqueous protein solution of the same concentration.

The detailed description above describes a protein aqueous suspension preparation, a method of preparing a protein aqueous suspension preparation and a prefilled syringe. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

### DESCRIPTION OF REFERENCE SYMBOLS

1 Prefilled syringe
2 Sheath
3, 4 Gasket
5 Plunger
6 Protein-polyamino acid complex
7 Dissolving liquid (NaCl solution)
8 Enlarged diameter portion

What is claimed is:

1. A protein aqueous suspension preparation comprising a protein and a polyamino acid, said protein and said polyamino acid having a surface charge in a buffer and forming a complex suspended in the buffer, wherein the protein is at least one selected from the group consisting of human serum albumin, bovine serum albumin, human atrial natriuretic peptide, bovine thyroglobulin, anti-IgE monoclonal antibody, anti-EGFR monoclonal antibody, human soluble TNF receptor-Fc fusion protein, human IgG, darbepoetin α, and anti-TNFα monoclonal antibody Fab fragment.

2. The protein aqueous suspension preparation according to claim 1, wherein the polyamino acid is an anionic polyamino acid.

3. The protein aqueous suspension preparation according to claim 2, wherein the anionic polyamino acid is at least one selected from group consisting of polyglutamic acid, polyaspartic acid, and their water-soluble salts.

4. The protein aqueous suspension preparation according to claim 1, wherein the polyamino acid is a cationic polyamino acid.

5. The protein aqueous suspension preparation according to claim 4, wherein the cationic polyamino acid is at least one selected from group consisting of polylysine, polyarginine, polyhistidine, and their water-soluble salts.

6. The protein aqueous suspension preparation according to claim 1, wherein a molecular weight of the polyamino acid is in a range from 0.5 kDa to 1000 kDa.

7. The protein aqueous suspension preparation according to claim 1, wherein a molecular weight of the protein is in a range from 3 kDa to 670 kDa.

8. A method of preparing a protein aqueous suspension preparation comprising forming a complex of a protein having a surface charge and a polyamino acid having a surface charge in a buffer, wherein the protein is at least one selected from the group consisting of human serum albumin, bovine serum albumin, human atrial natriuretic peptide, bovine thyroglobulin, anti-IgE monoclonal antibody, anti-EGFR monoclonal antibody, human soluble TNF receptor-Fc fusion protein, human IgG, darbepoetin α, and anti-TNFα monoclonal antibody Fab fragment, and removing water or buffer from the protein aqueous suspension preparation to increase the concentration of the protein in the protein aqueous suspension preparation.

9. The method of preparing a protein aqueous suspension preparation of claim 8, wherein the removal of water or buffer increases the concentration of the protein from 3 to 10 times the original concentration in the protein aqueous suspension preparation.

10. A prefilled syringe comprising an axially extending sheath possessing a proximal end and a distal end, the sheath possessing an interior divided into a first chamber and a second chamber by a slidable gasket positioned axially between the first and second chambers, and a plunger possessing a distal end portion positioned in the interior of the sheath at a position proximal of the gasket, the plunger being axially movable relative to the sheath in a distal direction, the first chamber containing a protein aqueous suspension preparation comprised of a protein and a polyamino acid, said protein and said polyamino acid having a surface charge in a buffer and forming a complex suspended in the buffer, wherein the protein aqueous suspension preparation has been concentrated by removal of water or buffer, the second chamber containing an aqueous electrolyte solution, the sheath being configured so that axial movement of the plunger in the distal direction causes the first and second chambers to communicate with one another so that the protein aqueous suspension of the first chamber and the aqueous electrolyte solution of the second chamber mix together to dissolve the complex and produce a resulting protein solution that is dispensable from the distal end of the sheath, and wherein the protein is at least one selected from the group consisting of human serum albumin, bovine serum albumin, human atrial natriuretic peptide, bovine thyroglobulin, anti-IgE monoclonal antibody, anti-EGFR monoclonal antibody, human soluble TNF receptor-Fc fusion protein, human IgG, darbepoetin α, and anti-TNFα monoclonal antibody Fab fragment.

11. The prefilled syringe of claim 10, wherein the sheath includes an enlarged diameter section so that when the gasket axially overlaps the enlarged diameter section, the first and second chambers communicate with one another so that the protein aqueous suspension and the aqueous electrolyte solution are mixed to dissolve the complex.

12. The prefilled syringe of claim 10, wherein the first chamber is closer to the distal end of the sheath and the second chamber is closer to the proximal end of the sheath.

13. The prefilled syringe of claim 10, wherein the aqueous electrolyte solution is an aqueous solution of sodium chloride.

14. The prefilled syringe of claim 10, wherein the polyamino acid is at least one selected from group consisting of polylysine, polyarginine, polyhistidine, polyglutamic acid, polyaspartic acid, and their water-soluble salts.
